(19) United States
(12) Patent Application Publication
(10) Pub. No.: US 2009/0197883 A1
(43) Pub. Date: Aug. 6, 2009

(54) Diphenyl Substituted Cycloalkanes, Compositions Containing Such Compounds and Methods Of Use

(76) Inventors: Helen M. Armstrong, Westfield, NJ (US); Linda L. Chang, Wayne, NJ (US); Richard Frenette, Laval (CA); Dwight MacDonald, I'lle Bizard (CA); Hyun O. Ok, Colonia, NJ (US); Michel Therien, Laval (CA); Feroze Ujjainwalla, Scotch Plains, NJ (US)

Correspondence Address: MERCK AND CO., INC, P O BOX 2000, RAHWAY, NJ, 07065-0907, US

(21) Appl. No.: 11/885,851

(22) PCT Filed: Mar. 3, 2006
(86) PCT No.: PCT/US06/07717

§ 371 Date: Jun. 30, 2008

Related U.S. Application Data
(60) Provisional application No. 60/659,898, filed on Mar. 9, 2005.

(51) Int. Cl. A61K 31/5377; C07D 417/02; A61K 31/4439; C07D 401/02; A61K 31/4709; C07D 413/02; C07D 403/02; A61K 31/506; C07D 487/02; A61K 31/519; A61P 29/00; A61P 9/10

(52) U.S. Cl. 514/235.2; 546/270.1; 514/338; 546/167; 514/314; 544/128; 544/331; 514/275; 544/263; 514/262.1

(57) ABSTRACT

The instant invention provides compounds of Formula (I) which are 5-lipoxygenase activating protein inhibitors. Compounds of Formula (I) are useful as anti-atherosclerotic, anti-asthmatic, anti-allergic, anti-inflammatory and cytoprotective agents.

### FIELD OF THE INVENTION

The instant invention involves compounds that inhibit 5-lipoxygenase activating protein (FLAP), compositions containing such compounds and methods of treatment using such compounds for the treatment and prevention of atherosclerosis and related diseases and conditions.

### BACKGROUND OF THE INVENTION

Inhibition of leukotriene biosynthesis has been an active area of pharmaceutical research for many years. Leukotrienes are potent contractile and inflammatory mediators derived through the oxygenation of arachidonic acid by 5-lipoxygenase.

One class of leukotriene biosynthesis inhibitors are those known to act through inhibition of 5-lipoxygenase (5-LO). In general, 5-LO inhibitors have been sought for the treatment of allergic rhinitis, asthma and inflammatory conditions including arthritis. One example of a 5-LO inhibitor is the marketed drug zileuton, which is indicated for the treatment of asthma. More recently, it has been reported that 5-LO may be an important contributor to the atherogenic process; see Mehrabian, M. et al., Circulation Research, 2002 Jul. 26, 91(2):120-126.

A new class of leukotriene biosynthesis inhibitors (now known as FLAP inhibitors) distinct from 5-LO inhibitors is described in Miller, D. K. et al., Nature, vol. 343, No. 6255, pp. 278-281, 18 Jan. 1990. These compounds inhibit the formation of cellular leukotrienes but have no direct effect on soluble 5-LO activity. These compounds were used to identify and isolate the inner nuclear membrane 18,000 dalton protein 5-lipoxygenase-activating protein (FLAP). In cells, arachidonic acid is released from membrane phospholipids by the action of cytosolic phospholipase 2. This arachidonic acid is transferred to nuclear membrane bound 5-lipoxygenase by FLAP. The presence of FLAP in cells is essential for the synthesis of leukotrienes. Additionally, based on studies described in Helgadottir, A., et al., Nature Genetics, vol 36, no. 3 (March 2004) 233-239, it is believed that the gene encoding 5-lipoxygenase activating protein confers risk for myocardial infarction and stroke in humans.

Despite significant therapeutic advances in the treatment and prevention of atherosclerosis and ensuing atherosclerotic disease events, such as the improvements that have been achieved with HMG-CoA reductase inhibitors, further treatment options are clearly needed. The instant invention addresses that need by providing compounds, compositions and methods for the treatment or prevention of atherosclerosis as well as related conditions.

### SUMMARY OF THE INVENTION

The instant invention relates to compounds of Formula I which are FLAP inhibitors, methods for their preparation, and methods and pharmaceutical formulations for using these compounds. in mammals, especially humans. This invention provides compounds of structural Formula I:

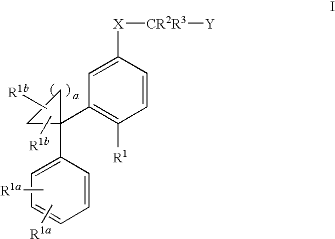

and the pharmaceutically acceptable salts, esters and solvates thereof. This invention also involves the use of compounds described herein to slow or halt atherogenesis. Therefore, one object of the instant invention is to provide a method for treating atherosclerosis, which includes halting or slowing the progression of atherosclerotic disease once it has become clinically evident, comprising administering a therapeutically effective amount of a compound of Formula I to a patient in need of such treatment. Another object is to provide methods for preventing or reducing the risk of developing atherosclerosis and atherosclerotic disease events, comprising administering a prophylactically effective amount of a compound of Formula I to a patient who is at risk of developing atherosclerosis or having an atherosclerotic disease event.

The compounds of Formula I are also useful as anti-asthmatic, anti-allergic, anti-inflammatory and cytoprotective agents. They are also useful in treating angina, cerebral spasm, glomerular nephritis, hepatitis, endotoxemia, uveitis, and allograft rejection. The instant invention provides methods of treatment comprising administering a therapeutically effective amount of a compound of Formula I to a patient in need of the above-described treatments.

A further object is to provide the use of FLAP inhibitors of Formula I in combination with other therapeutically effective agents, including other anti-atherosclerotic drugs. These and other objects will be evident from the description contained herein.

### DETAILED DESCRIPTION OF THE INVENTION

The instant invention provides a compound represented by structural Formula I

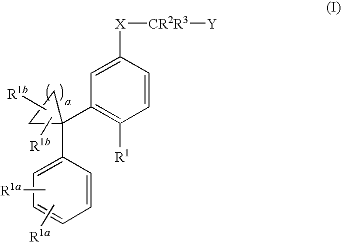

and the pharmaceutically acceptable salts, esters and solvates thereof wherein:

a is an integer selected from 1, 2, 3 and 4;

each R^{1a} is independently selected from the group consisting of: —H, —F, —Cl, —Br, —C₁₋₆alkyl, —CN, —OH, C₁₋₆alkyl-OH, —OC₁₋₆ alkyl, -fluoroC₁₋₆ alkyl, -fluoroC₁₋₆ alkoxy, —NH₂, —NHC₁₋₆alkyl, —N(C₁₋₆alkyl)₂, —C₁₋₆alkyl-NH₂, —C₁₋₆alkyl-NHC₁₋₆alkyl, —C₁₋₆alkyl-N(C₁₋₆alkyl)₂, —NHC(O)C₁₋₆alkyl, —CO₂C₁₋₆alkyl, —C(O)NHC₁₋₆alkyl and —C(O)N(C₁₋₆alkyl)₂;

each R^{1b} is independently selected from the group consisting of: —H, —F, —C₁₋₆ alkyl, —OH, —OC₁₋₆ alkyl, -fluoroC₁₋₆alkyl, -fluoroC₁₋₆alkoxy, —N(R^{a})₂ and —C₁₋₁₆alkyl-N(R^{a})₂,

or one R^{1b} group can represent oxo and the other is as previously defined;

R¹ is selected from the group consisting of:

a) Z¹,

b) —CO₂R^{a}, C(O)NR^{a}R^{b}, —N(R^{a})₂, —NR^{b}SOₚR^{a}, —NR^{b}C(O)R^{a}, NR^{b}C(O)NR^{a}R^{b}, —NR^{b}CO₂R^{a}, OC(O)NR^{a}R^{b}, —OH and —CN,

c) —C₁₋₆alkyl, —C₂₋₆alkenyl, —C₂₋₆alkyl, —OC₁₋₆alkyl, —OC₂₋₆alkenyl and —OC₂₋₆alkynyl, said groups being optionally substituted with R⁴ and optionally substituted with R⁵,

wherein R⁴ is selected from the group consisting of: —CO₂R^{a}, —C(O)NR^{a}R^{b}, —N(R^{a})₂, —NR^{b}SOₚR^{a}, —NR^{b}C(O)R^{a}NR^{b}C(O)NR^{a}R^{b}, —NR^{b}CO₂R^{a}, —OC(O)NR^{a}R^{a}, —C(O)SOₚNR^{a}R^{b}, —C(O)NR^{b}NR^{a}R^{b}, —S(O)ₚNR^{a}R^{b}, —SOₚNR^{b}C(O)R^{a}, —S(O)ₚR^{a}, —F, —CF₃, phenyl, Hetcy and Z¹; and R⁵ is selected from the group consisting of —F and —H, and

d) phenyl, optionally substituted with 1-2 members selected from the group consisting of: —F, —Cl, —C₁₋₁₆alkyl, —CN, —OH, —OC₁₋₆alkyl, -fluoroC₁₋₆alkyl, -fluoroC₁₋₆alkoxy, —NH₂, —NHC₁₋₆alkyl, —N(C₁₋₆alkyl)₂, —C₁₋₆alkyl-N—, —C₁₋₆alkyl-NHC₁₋₆alkyl, —C₁₋₆alkyl-N(C₁₋₆alkyl)₂, —C₁₋₆alkyl-CN, —NHC(O)C₁₋₆alkyl, —C(O)NHC₁₋₆alkyl and —C(O)N(C₁₋₆alkyl)₂;

R² is selected from the group consisting of —H and —C₁₋₆alkyl optionally substituted with a group selected from —OH and —F;

R³ is selected from the group consisting of —H and —C₁₋₆alkyl;

each “p” independently represents an integer selected from 0, 1 and 2;

each R^{a} is independently selected from the group consisting of

a) —H,

b) —C₁₋₄alkyl, —C₂₋₄alkenyl and —C₂₋₄alkynyl, wherein each is optionally substituted with 1-2 members selected from the group consisting of: —OH, —OC₁₋₄alkyl, —CN, —NH₂, —NHC₁₋₄alkyl, and —N(C₁₋₄alkyl)₂, —F and —CF₃,

c) phenyl and phenyl-C₁₋₄alkyl-, the phenyl moieties being optionally substituted with 1-2 members selected from the group consisting of: —F, —Cl, —C₁₋₄ alkyl, —CN, —OH, —OC₁₋₄ alkyl, -fluoroC₁₋₄alkyl, -fluoroC₁₋₄alkoxy, —NH₂, —NHC₁₋₄alkyl, —N(C₁₋₄alkyl)₂, —C₁₋₄alkyl-NH₂, —C₁₋₄alkyl-NHC₁₋₄alkyl, —C₁₋₄alkyl-N(C₁₋₄alkyl)₂, —C₁₋₄alkyl-CN, —NHC(O)C₁₋₄alkyl, —C(O)NHC₁₋₄alkyl and —C(O)N(C₁₋₄alkyl)₂,

and the alkyl portion of phenyl-C₁₋₄alkyl- being optionally substituted with —OH, —CN, —OC₁₋₄alkyl, —NH₂, —NHC₁₋₄alkyl, —N(C₁₋₄alkyl)₂, and 1-3 of fluoro,

d) Hetcy and Hetcy-C₁₋₄alkyl-, the Hetcy moieties being optionally substituted on carbon with 1-2 members selected from the group consisting of —F, —OH, —CO₂H, —C₁₋₄alkyl, —CO₂C₁₋₄alkyl, —OC₁₋₄alkyl, —NH₂, —NHC₁₋₄alkyl, —N(C₁₋₄alkyl)₂, —NHC(O)C₁₋₄alkyl, oxo, —C(O)NHC₁₋₄alkyl and —C(O)N(C₁₋₄alkyl)₂; and optionally substituted on nitrogen when present with a group selected from —C₁₋₄alkyl and —C₁₋₄acyl,

and the alkyl portion of Hetcy-C₁₋₄alkyl- being optionally substituted with a member selected from the group consisting of —OH, —CN, —OC₁₋₄alkyl, —NH₂, —NHC₁₋₄alkyl, —N(C₁₋₄alkyl)₂ and 1-3 of fluoro,

e) Z² and Z²-C₁₋₄alkyl- and the alkyl portion of Z²-C₁₋₄alkyl- being optionally substituted with a member selected from the group consisting of —OH, —CN, —OC₁₋₄alkyl, —NH₂, —NHC₁₋₄alkyl, —N(C₁₋₄alkyl)₂ and 1-3 of fluoro;

each R^{b} is independently selected from the group consisting of —H and —C₁₋₃alkyl optionally substituted with 1-2 members selected from the group consisting of NH₂, —OH, —F, —CN and —CF₃;

X is selected from the group consisting of O— and —CHR⁶—, wherein R⁶ is selected from the group consisting of —H, —OH and —C₁₋₆alkyl optionally substituted with a group selected from —OH and —F;

Y is selected from the group consisting of:

a) a 9-membered unsaturated ortho-fused bicyclic ring system containing 2-3 heteroatoms selected from the group consisting of —N═, —NH—, —N(Me)—, —S— and —O—, and wherein the ring system is optionally substituted with 1-3 of fluoro,

b) a 10-membered aromatic ortho-fused bicyclic ring system containing 1-3 of —N═, wherein the ring system is optionally substituted with 1-3 of fluoro, and

c) pyridinyl substituted with a group selected from —C₁₋₄alkyl, —F, —CF₂H and CF₃, and optionally having a second substituent which is —C₁₋₄alkyl;

Hetcy is selected from the group consisting of azetidinyl, pyrrolidinyl, piperidinyl, piperazinyl, morpholinyl, tetraydrofuranyl and β-lactamyl, δ-lactamyl and γ-lactamyl;

Z¹ is selected from the group consisting of:

a) a 5-membered unsaturated heterocyclic ring containing 2-4 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and —C₁₋₄alkyl substituted with a group selected from —NH₂, —OH, —OC₁₋₁₄alkyl, —CN and 1-3 of fluoro,

b) a 5-membered unsaturated heterocyclic ring containing 2-3 heteroatoms selected from one oxygen or one sulfur and 1-2 of nitrogen, wherein one nitrogen in the ring is optionally substituted with a group selected from C₁₋₄alkyl and C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro,

c) a 6-membered unsaturated heterocyclic ring containing 1-2 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, —S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro,

d) an 8-membered unsaturated ortho-fused bicyclic ring system containing 3-5 heteroatoms selected from one sulfur and 2-4 of nitrogen wherein one carbon in the ring is optionally substituted with a group selected from ═O, ═S, —SMe, —NH₂, —CF₃, —Cl, —Cl₄alkyl and C₁₋₄alkyl substituted with a group selected from —NH₂, —OH, —OC₁₋₄alkyl, —CN and 1-3 of fluoro, and

e) a 9-membered unsaturated ortho-fused bicyclic ring system containing 3-4 nitrogen atoms, wherein one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro; and

Z² is selected from the group consisting of:

a) a 5-membered unsaturated heterocyclic ring containing 2-4 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro,

b) a 5-membered unsaturated heterocyclic ring containing 2-3 heteroatoms selected from one oxygen or one sulfur and 1-2 of nitrogen, wherein one nitrogen in the ring is optionally substituted with a group selected from C₁₋₄alkyl and C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, and C₁₋₄alkyl optionally substituted with a group selected from —NH₂, —OH, —OC₁₋₄alkyl, —CN and 1-3 of fluoro, and

c) a 6-membered unsaturated heterocyclic ring containing 1-2 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from —O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro.

The invention is described herein in detail using the terms defined below unless otherwise specified. “Alkyl”, as well as other groups having the prefix “alk”, such as alkoxy, alkanoyl and the like, means carbon chains which may be linear, branched or cyclic, or combinations thereof, containing the indicated number of carbon atoms. “Non-cyclic alkyl” is a subset of alkyl and means linear and branched alkyl, and does not include cycloalkyl. If no number is specified, 1-10 carbon atoms are intended for linear or branched alkyl groups. Cycloalkyl, which must have a minimum of 3 carbons to form a carbocyclic ring, is a subset of alkyl and is also intended to be included within the meaning of “alkyl” when the specified number of carbon atoms for an alkyl group encompasses three or more carbon atoms, or when no number of carbon atoms is specified. As a result, each occurrence of the term “alkyl” independently represents the group consisting of (a) non-cyclic alkyl, (b) cycloalkyl and (c) a combination of non-cyclic alkyl with cycloalkyl. Therefore, it is understood that when “C₁₋₃alkyl” is recited, this encompasses linear and branched 1-3 carbon chains and cyclopropyl. Similarly, when “C₁₋₄alkyl” is recited, this encompasses linear and branched 1-4 carbon chains as well as cyclopropyl, —CH₂-cyclopropyl, -cyclopropyl-CH₃ and cyclobutyl. Similarly, when “C₁₋₆ alkyl” is recited, this encompasses linear and branched 1-6 carbon chains and C₃₋₆ cycloalkyl, as well as combinations of non-cyclic alkyl with C₃₋₅cycloalkyl which contain a total up to of six carbon atoms. Examples of alkyl groups include but are not limited to methyl, ethyl, propyl, isopropyl, butyl, sec- and tert-butyl, 1,1-dimethylbutyl, pentyl, isopentyl, hexyl, heptyl, octyl, nonyl and the like, as well as the cycloalkyl groups cyclopropyl, cyclobutyl, cyclopentyl and cyclohexyl. Cyclopropyl and cyclobutyl are preferred cycloalkyl groups.

“Alkenyl” means carbon chains which contain at least one carbon-carbon double bond, and which may be linear or branched or combinations thereof, containing the indicated number of carbon atoms, and more particularly 3-6 carbons. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof, containing the indicated number of carbon atoms, and more particularly 3-6 carbons. Examples of alkynyl include ethynyl, propargyl, 3-methyl-1-pentynyl, 2-heptynyl and the like.

“Acyl” refers to an alkyl group as defined above linked through a carbonyl group. A preferred example is acetyl, CH₃C(O)—. “Aryl” (Ar) means mono- and bicyclic aromatic rings containing 6-12 carbon atoms. Examples of aryl include phenyl, naphthyl, indenyl and the like. “Halogen” (Halo) includes fluoro, chloro, bromo and iodo, preferably —F and —Cl, more preferably —F.

The phrase “8-membered unsaturated ortho-fused bicyclic ring system” as used herein means a 5 membered ring fused to a 5-membered ring wherein the rings have two, and only two, adjacent atoms in common, i.e., they are ortho-fused. The phrase “9-membered unsaturated ortho-fused bicyclic ring system” as used herein means a 6 membered ring and a 5-membered ring ortho-fused together. The phrase “10-membered aromatic ortho-fused bicyclic ring system” as used herein means two 6-membered rings ortho-fused together. Said bicyclic ring systems are comprised of carbon atoms and the indicated number and kind of heterotaoms, and may be substituted as defined herein. The term “unsaturated” encompasses both aromatic rings as well as non-aromatic unsaturated rings.

“Hetcy” can be linked to a compound of structural Formula I via carbon or nitrogen in the Hetcy ring. Each of “Z¹” and “Z²” can be linked to a compound of structural Formula I via carbon or nitrogen in the Z¹ or Z² ring or ring system, and is preferably linked via carbon. “Y” can be linked to a compound of structural Formula I via carbon or nitrogen in the Y ring or ring system, and is preferably linked via carbon.

The term “optionally substituted” means “unsubstituted or substituted,” and therefore, the genus described herein encompasses compounds containing the specified optional substituent as well as compounds that do not contain the optional substituent. For example, the phrase “—C₁₋₃alkyl optionally substituted with a group selected from —OH and —F″ encompasses unsubstituted —C₁₋₃alkyl, fluoro substituted —C₁₋₃alkyl and hydroxy substituted —C₁₋₃alkyl.

Reference to the compounds of this invention as those of “Formula I,” “Formula Ia,” “Formula Ib,” or any other generic structural formulas depicted herein, is intended to encompass compounds falling within the scope of each of these structural formulas including pharmaceutically acceptable salts, esters and solvates thereof where such salts, esters and solvates are possible. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like, and particularly citric, fumaric, hydrobromic, hydrochloric, maleic, phosphoric, sulfuric, and tartaric acids.

Pharmaceutically acceptable esters of available hydroxy or carboxylic acid groups can optionally be formed as well. Examples of pharmaceutically acceptable esters include, but are not limited to, —C₁₋₄ alkyl and —C₁₋₄ alkyl substituted with phenyl-, dimethylamino- and acetylamino.

The compounds of Formula I may contain one or more asymmetric centers, and can thus occur as racemates, racemic mixtures, single enantiomers, diastereoisomeric mixtures and individual diastereoisomers. The present invention in all its embodiments includes all such isomers, as well as salts, esters and solvates of such racemates, mixtures, enantiomers and diastereoisomers. Furthermore, some of the crystalline forms of compounds of the present invention may exist as polymorphs and as such are intended to be included in the present invention. In addition, some of the compounds of the instant invention may form solvates with water or common organic solvents. Such solvates and hydrates are likewise encompassed within the scope of this invention. Some of the compounds described herein contain olefinic double bonds. The invention includes both E and Z geometric isomers. Some of the compounds described herein may exist as tautomers, e.g., keto-enol tautomers. Individual tautomers as well as mixtures thereof are included in the present invention.

Compounds of structural Formula I may be separated into their individual diastereoisomers by, e.g., fractional crystallization from suitable solvents, e.g., methylene chloride/hexanes or ethyl acetate/hexanes, or via chiral chromatography using an optically active stationary phase. Absolute stereochemistry may be determined by X-ray crystallography of crytalline products or crystalline intermediates which are derivatized, if necessary, with a reagent containing a stereogenic center of known configuration. Alternatively, any stereoisomer of a compound of the general Formula I may be obtained by stereospecific synthesis using optically pure starting materials or reagents of known absolute configuration.

In an embodiment of this invention are compounds within the scope of Formula I having structural Formula Ia:

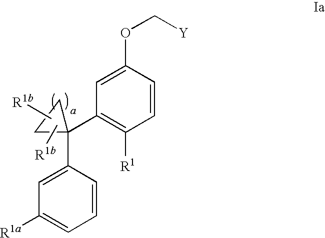

and the pharmaceutically acceptable salts, esters and solvates thereof wherein R¹, R^{1a}, R^{1b}, a and Y are as defined in Formula I.

In another embodiment of this invention are compounds within the scope of Formula I and Formula Ia, having structural Formula Ib:

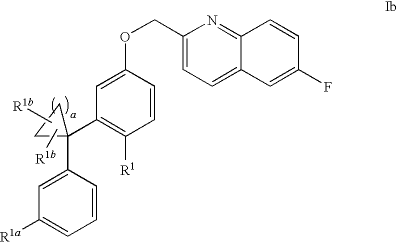

and the pharmaceutically acceptable salts, esters and solvates thereof wherein R¹, R^{1a}, R^{1b}, and a are as defined in Formula I.

In another embodiment of this invention are compounds of Formula I, Ia and Ib, wherein a is as defined above in Formula I. In a class of this embodiment, a is selected from 2, 3 and 4. In a sub-class of this embodiment, a is 2.

In another embodiment of this invention are compounds of Formula I, Ia and Ib, wherein R^{1a} is as defined above in Formula I. In a class of this embodiment, each R^{1a} is independently selected from —H and —F. In a sub-class of this embodiment R^{1a} is —H.

In another embodiment of this invention are compounds of Formula I, Ia and Ib, wherein R^{1b} is as defined above in Formula I. In a class of this embodiment, each R^{1b} is independently selected from —H and —CH₃.

In another embodiment of this invention are compounds of Formula I, Ia and Ib, wherein

R¹ is as defined in Formula I. In a class of this embodiment, R¹ is selected from —COOH, —COOC₁₋₆alkyl, —C(O)—NR^{a}R^{b}, —OC(O)—NR^{a}R^{b}, —CH₂C(O)—NR^{a}R^{b} and Z¹. In a sub-class of this embodiment, R¹ is selected from —C(O)—NR^{a}R^{b}, —OC(O)—NR^{a}R^{b} particularly —OC(O)—N(H)-pyridin-3-yl, and Z¹. In a further sub-class. R¹ is selected from

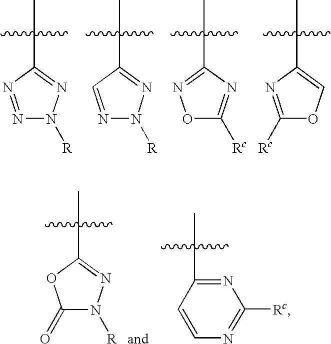

wherein R is selected from —H, —C₁₋₄alkyl and —C₁₋₄alkyl substituted with a group selected from —NH₂, —OH, —CN and 1-3 of fluoro, and particularly R is selected from —H, methyl and ethyl and -fluoroethyl; and R^{c} is selected from —H, ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro, and particularly R^{c} is selected from —H, methyl, —NH₂, ═O, -hydroxyethyl, fluoroethyl and 1-methyl-1-hydroxyethyl.

### Particularly, R¹ is

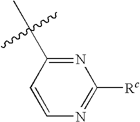

and more particularly it is

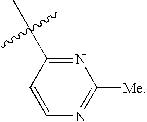

In another embodiment of this invention, R² is as defined above in Formula I. In a class of this embodiment, R² is —H.

In another embodiment of this invention, R³ is as defined above in Formula I. In a class of this embodiment, R³ is —H.

In another embodiment of this invention are compounds of Formula I, Ia and Ib, wherein R⁴ is as defined above in Formula I. In a class of this embodiment, R⁴ is selected from —H, —CONR^{a}R^{b}, —OCONR^{a}R^{b}, —CO₂C₁₋₆alkyl and Z¹.

In another embodiment of this invention are compounds of Formula I, Ia and Ib, wherein R⁵ is as defined above in Formula I.

In another embodiment of this invention are compounds of Formula I, Ia and Ib, wherein “p” is an integer selected from 0, 1 and 2, and particularly p is 2.

In another embodiment of this invention are compounds of Formula I, Ia and Ib, wherein R^{a} is as defined above in Formula I. In a class of this embodiment, R^{a} is selected from —H and Z². In a sub-class of this embodiment, R^{a} is selected from pyridinyl, particularly pyridin-3-yl, pyrimidinyl, pyrazinyl, thiazolyl, thiadiazolyl, triazolyl and pyrazolyl. In a further sub-class of this embodiment, R^{a} is selected from

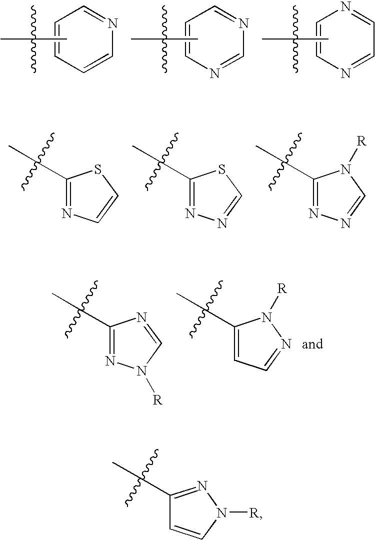

wherein R is as defined above.

In another embodiment of this invention are compounds of Formula I, Ia and Ib, wherein R^{b} is as defined above in Formula I. In a class of this embodiment, R^{b} is selected from —H, methyl, ethyl, propyl and iso-propyl. In a sub-class of this embodiment R^{b} is —H and methyl.

In another embodiment of this invention, X is as defined above in Formula I. In a class of this embodiment, X is —O—.

In another embodiment of this invention are compounds of Formula I and Ia wherein Y is is as defined in Formula I. In a class of this embodiment, Y is selected from:

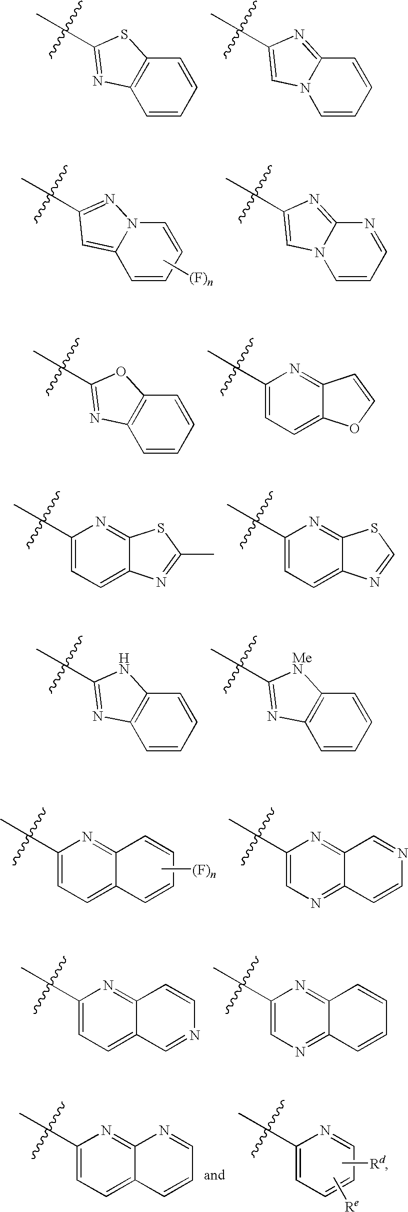

wherein R^{d} is selected from —C₁₋₄alkyl, —F, —CF₂H and —CF₃; R^{e} is selected from —H and —C₁₋₄alkyl; and n is an integer selected from zero, 1, 2 and 3. In a sub-class of this embodiment, Y is selected from:

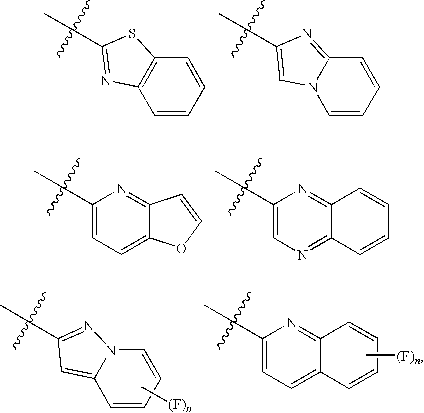

n is an integer selected from 1, 2 and 3
particularly

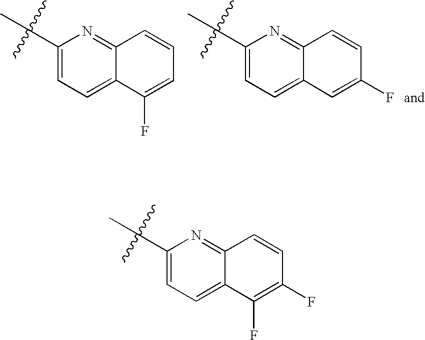

In yet a further sub-class of this embodiment, Y is

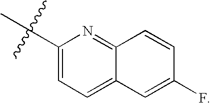

In another embodiment of this invention are compounds of Formula I, Ia and Ib, wherein Hetcy is as defined in Formula I. In a class of this embodiment, Hetcy is selected from pyrrolidinyl and piperidinyl, each member being optionally substituted as defined in Formula I.

In another embodiment of this invention are compounds of Formula I, Ia and Ib, wherein Z¹ is as defined in Formula I. In a class of this embodiment, Z¹ is selected from:

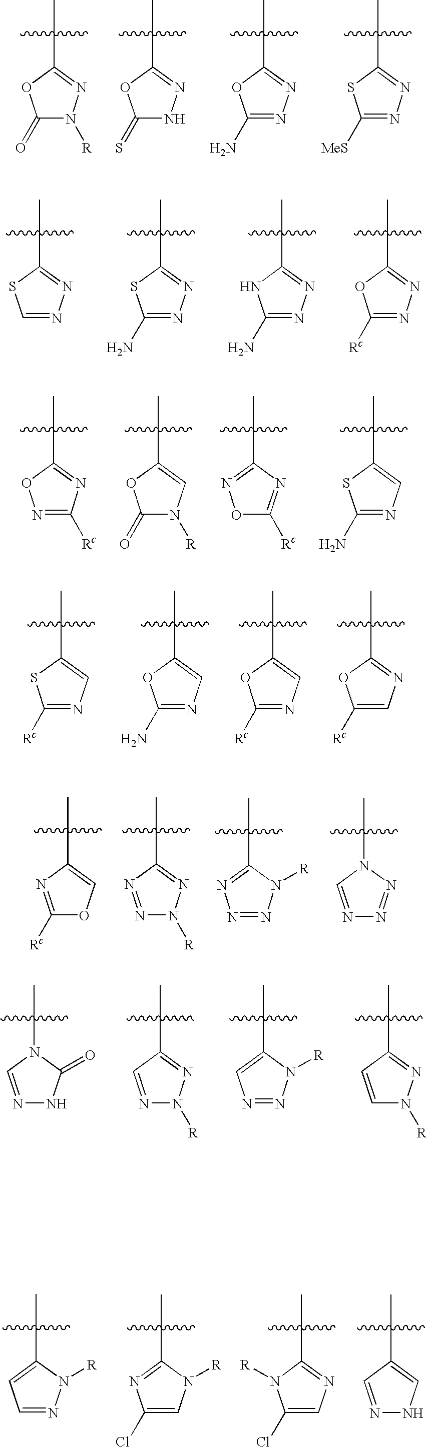

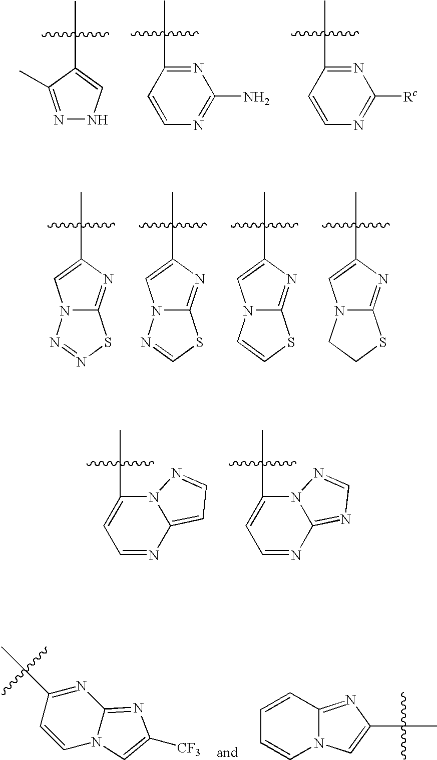

wherein R is selected from —H, —C₁₋₄alkyl and —C₁₋₄alkyl substituted with a group selected from —NH₂, —OH, —CN and 1-3 of fluoro, and particularly R is selected from —H, methyl and ethyl and -fluoroethyl; and R^{c} is selected from —H, ═O, ═S, —SMe, —NH₂, —CF₃, —Cl, —C₁₋₄alkyl and —C₁₋₄alkyl substituted with a group selected front —NH₂, —OH, —OC₁₋₄alkyl, —CN and 1-3 of fluoro, and particularly R^{c}C is selected from —H, methyl, —NH₂, —O, -hydroxyethyl, fluoroethyl and 1-methyl-1-hydroxyethyl. In a class of this embodiment, Z¹ is selected from

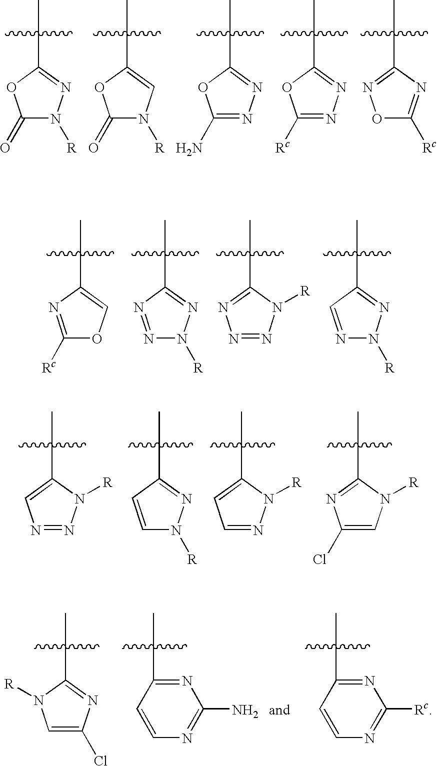

In a sub-class of this embodiment, Z¹ is selected from

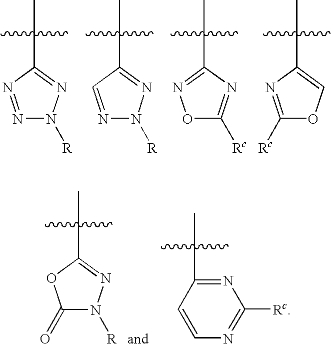

### Particularly, Z¹ is

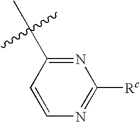

and more particularly it is

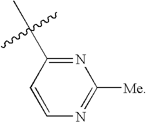

In another embodiment of this invention are compounds of Formula I, Ia and Ib, wherein Z² is as defined in Formula L. In a class of this embodiment, Z² is selected from pyridinyl, pyrimidinyl, pyrazinyl, thiazolyl, thiadiazolyl, triazolyl and pyrazolyl, each member being optionally substituted as defined in Formula I. In a further sub-class of this embodiment, Z² is selected from

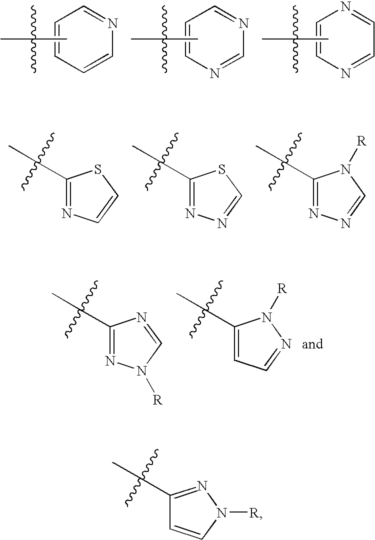

wherein R is as defined above.

In another embodiment of this invention are compounds of Formula I, Ia and Ib, wherein

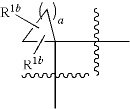

is selected from the group consisting of:

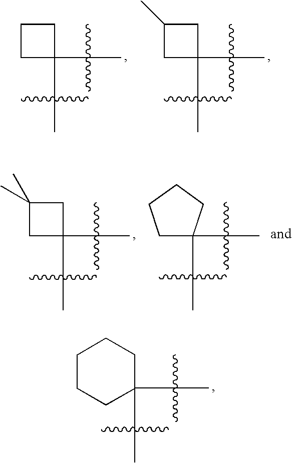

and more particularly, it is selected from

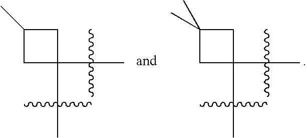

In a particular embodiment of this invention are compounds of Formula I wherein:

Y is selected from the group consisting of

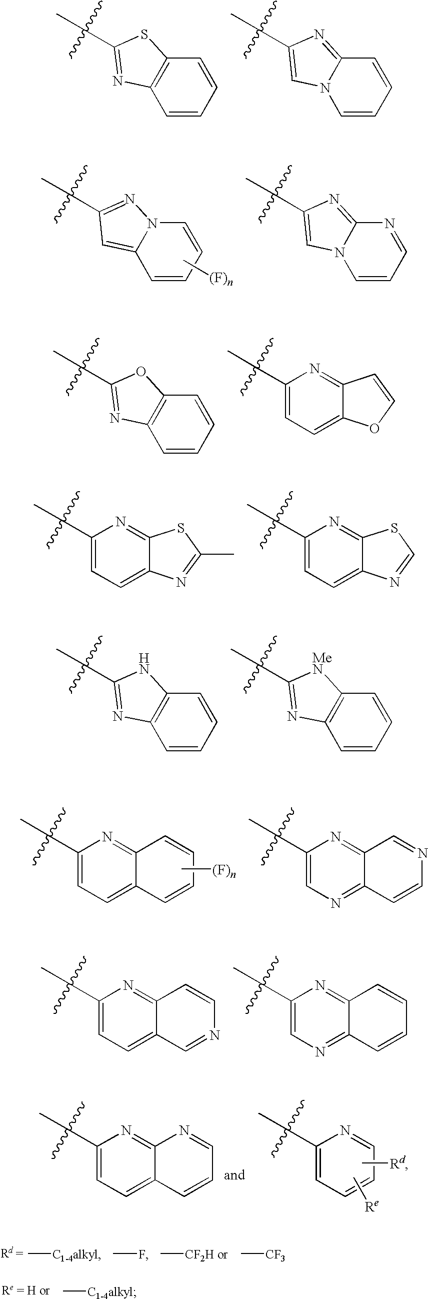

in a class thereof R¹ is selected from —COOH, —COOC₁₋₆alkyl, —C(O)—NR^{a}R^{b}, —OC(O)—NR^{a}R^{b}, —CH₂C(O)—NR^{a}R^{b} and Z¹; in a sub-class thereof X is —O—; in a further sub-class thereof. Z¹ is selected from the group consisting of:

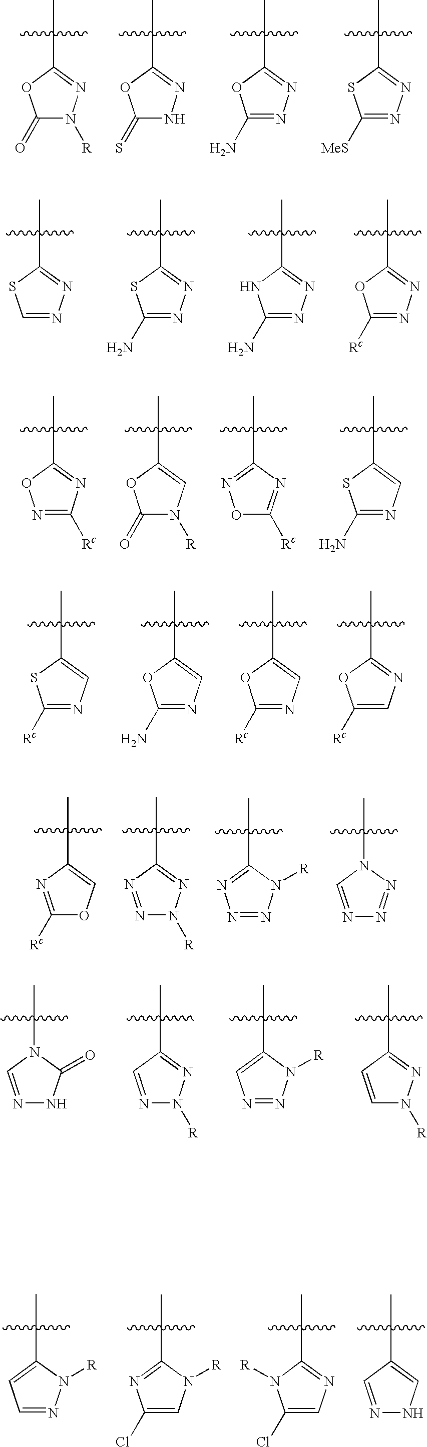

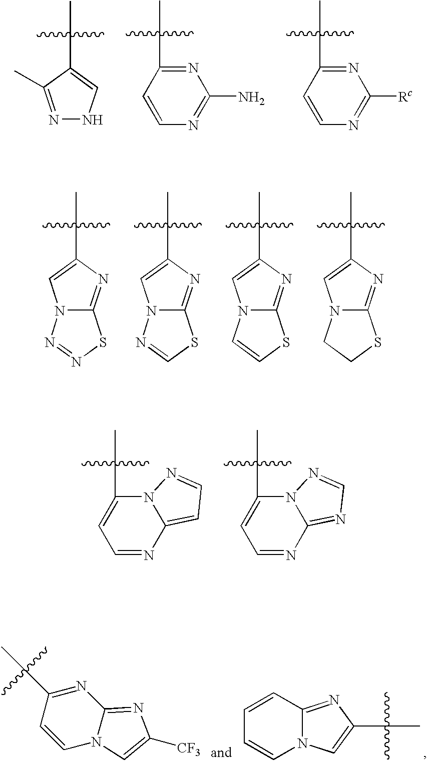

in a yet further sub-class thereof. R^{a} is selected from —I and Z², and R^{b} is selected from —H, methyl, ethyl, propyl and iso-propyl; in yet a further sub-class thereof Z² is selected from pyridinyl, pyrimidinyl, pyrazinyl, thiazolyl, thiadiazolyl, triazolyl and pyrazolyl; in a yet further sub-class thereof R⁴ is selected from —H, —CONR^{a}R^{b}, —OCONR^{a}R^{b}, —CO₂C₆alkyl and Z¹; in a yet further sub-class thereof a is selected from 2, 3 and 4; in a yet further subclass thereof each R^{1a} is independently selected from —H and —F; in yet a further subclass thereof each R^{1b} is independently selected from —H and —CH₃; in a yet further subclass thereof R² is —H and R³ is —H; and in a final sub-class thereof. Hetcy is selected from pyrrolidinyl and piperidinyl.

In a more particular embodiment are compounds of Formula Ia and Formula Ib wherein R^{1a} is selected from —H and —F; and

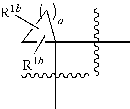

is selected from the group consisting of:

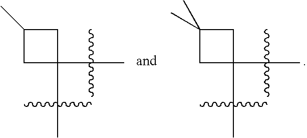

In a class of this embodiment R¹ is selected from —OC(O)NR^{a}R^{b} and Z¹, wherein Z¹ is selected from:
(a) a 5-membered unsaturated heterocyclic ring containing 2-4 nitrogen atoms, wherein one nitrogen in the ring is optionally substituted with a group selected from —C₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro,
(b) a 5-membered unsaturated heterocyclic ring containing 2-3 heteroatoms selected from one oxygen or one sulfur and 1-2 of nitrogen, wherein one nitrogen in the ring is optionally substituted with a group selected from C₁₋₄alkyl and C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, and C₁₋₄alkyl optionally substituted with a group selected from —NH₂, —OH, —OC₁₋₄alkyl, —CN and 1-3 of fluoro, and
(c) a 6-membered unsaturated heterocyclic ring containing 1-2 nitrogen atoms, wherein one nitrogen in the ring is optionally substituted with a group selected from —C₁₋₄alkyl and —C₁₋₄alkyl substituted with a group selected from NH₂, —OH, —CN and 1-3 of fluoro, and one carbon in the ring is optionally substituted with a group selected from ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro. In a sub-class of this embodiment, R¹ is selected from:

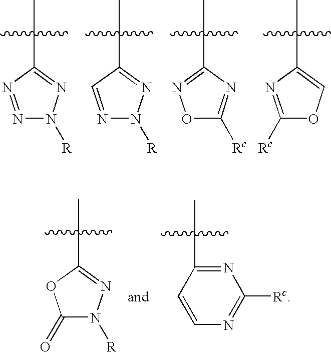

Examples of compounds that fall within the present invention include those shown in the examples contained herein, as well as salts and solvates thereof. When racemic mixtures are shown, the specific enantiomers are also included, as are the salts and solvates of the specific enantiomers.

The compounds of Formula I can be used for the treatment of atherosclerosis comprising administering a therapeutically effective amount of a compound of Formula I to a patient in need of such treatment. A further aspect of this invention involves a method for preventing or reducing the risk of developing atherosclerosis, comprising administering a prophylactically effective amount of a compound of Formula I to a patient in need of such treatment. Atherosclerosis is characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction, are all clinical manifestations of atherosclerosis and are therefore encompassed by the terms “atherosclerosis” and “atherosclerotic disease.”

A FLAP inhibitor may be administered to prevent or reduce the risk of occurrence, or recurrence where the potential exists, of a coronary heart disease event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a FLAP inhibitor to a patient at risk for such an event. The patient may already have atherosclerotic disease at the time of administration, or may be at risk for developing it.

The method of this invention particularly serves to prevent or slow new atherosclerotic lesion or plaque formation, and to prevent or slow progression of existing lesions or plaques, as well as to cause regression of existing lesions or plaques. Accordingly, one aspect of this invention involves a method for halting or slowing the progression of atherosclerosis, including halting or slowing atherosclerotic plaque progression, comprising administering a therapeutically effective amount of a FLAP inhibitor to a patient in need of such treatment. This method also includes halting or slowing progression of atherosclerotic plaques existing at the time the instant treatment is begun (i.e., “existing atherosclerotic plaques”), as well as halting or slowing formation of new atherosclerotic plaques in patients with atherosclerosis.

Another aspect of this invention involves a method for regression of atherosclerosis, including regression of atherosclerotic plaques existing at the time the instant treatment is begun, comprising administering a therapeutically effective amount of a FLAP inhibitor to a patient in need of such treatment. Another aspect of this invention involves a method for preventing or reducing the risk of atherosclerotic plaque rupture comprising administering a prophylactically effective amount of a FLAP inhibitor to a patient in need of such treatment.

The ability of the compounds of Formula I to inhibit biosynthesis of the leukotrienes makes them useful for preventing or reversing the symptoms induced by the leulkotrienes in a human subject. This inhibition of the mammalian biosynthesis of leukotrienes indicates that the compounds and pharmaceutical compositions thereof are useful to treat, prevent, or ameliorate in mammals and especially in humans: 1) pulmonary disorders including diseases such as asthma, chronic bronchitis, and related obstructive airway diseases, 2) allergies and allergic reactions such as allergic rhinitis, contact dermatitis, allergic conjunctivitis, and the like, 3) inflammation such as arthritis or inflammatory bowel disease, 4) pain, 5) skin disorders such as atopic eczema, and the like, 6) cardiovascular disorders such as angina, formation of atherosclerotic plaques, myocardial ischemia, hypertension, platelet aggregation and the like, 7) renal insufficiency arising from ischaemia induced by immunological or chemical (cyclosporin) etiology and 8) migraine or cluster headache, 9) ocular conditions such as uveitis, 10) hepatitis resulting from chemical, immunological or infectious stimuli, 11) trauma or shock states such as burn injuries, endotoxemia and the like, 12) allograft rejection, 13) prevention of side effects associated with therapeutic administration of cytokines such as Interleukin II and tumor necrosis factor, 14) chronic lung diseases such as cystic fibrosis, bronchitis and other small- and large-airway diseases, 15) cholecystitis, 16) multiple sclerosis, and 17) proliferation of myoblastic leukemia cells.

Thus, the compounds of the present invention may also be used to treat or prevent mammalian (especially, human) disease states such as erosive gastritis; erosive esophagitis; diarrhea; cerebral spasm; premature labor; spontaneous abortion; dysmenorrhea; ischemia; noxious agent-induced damage or necrosis of hepatic, pancreatic, renal, or myocardial tissue; liver parenchymal damage caused by hepatoxic agents such as CCl₄ and D-galactosamine; ischemic renal failure; disease-induced hepatic damage; bile salt induced pancreatic or gastric damage; trauma- or stress-induced cell damage; and glycerol-induced renal failure. The compounds also act as inhibitors of tumor metastasis and exhibit cytoprotective action.

The FLAP inhibitors of this invention can also be administered for prevention, amelioration and treatment of glomerulonephritis (see Guasch A., Zayas C. F., Badr K F. (1999), “MK-591 acutely restores glomerular size selectivity and reduces proteinuria in human glomerulonephritis,” Kidney Int., 56:261-267); and also for and prevention, amelioration and treatment of kidney damage resulting from diabetes complications (see Valdivielso J M, Montero A., Badr K F., Munger K A. (2003), “Inhibition of FLAP decreases proteinuria in diabetic rats,” J. Nephrol., 16(1):85-940.)

In addition, the compounds of this invention can also be used for the treatment of chronic obstructive pulmonary disease (COPD). As described in S. Kilfeather, Chest, 2002, vol 121, 197, airway neutrophilia in COPD patients is believed to be a contributing source of inflammation and is associated with airway remodeling. The presence of neutrophils is mediated in part by LTB₄, and treatment with the instant compounds could be used to reduce neutrophilic inflammation in patients with COPD.

The cytoprotective activity of a compound may be observed in both animals and man by noting the increased resistance of the gastrointestinal mucosa to the noxious effects of strong irritants, for example, the ulcerogenic effects of aspirin or indomethacin. In addition to lessening the effect of non-steroidal anti-inflammatory drugs on the gastrointestinal tract, animal studies show that cytoprotective compounds will prevent gastric lesions induced by oral administration of strong acids, strong bases, ethanol, hypertonic saline solutions, and the like, Two assays can be used to measure cytoprotective ability. These assays are: (A) an ethanol-induced lesion assay and (B) an indomethacin-induced ulcer assay and are described in EP 140,684.

In particular, the compounds of the invention would be useful to reduce the gastric erosion caused by co-administration of a cyclooxygenase-2 selective inhibitor and low-dose aspirin. Cyclooxygenase-2 selective inhibitors are widely used as effective anti-inflammatory drugs with less potential for gastrointestinal complications as compared to traditional, non-selective non-steroidal anti-inflammatory drugs. However, the combined use of a cyclooxygenase-2 selective inhibitor with low-dose aspirin for cardio protection may compromise the gastrointestinal safety of this class of compounds. By virtue of its activity as a 5-lipoxygenase inhibitor, the compounds of the invention would be expected to be gastric protective in this regard. See Fiorucci, et al. FASEB J. 17:1171-1173, 2003. Cyclooxygenase-2 selective inhibitors for use with the invention include but are not limited to etoricoxib (ARCOXIA™), celecoxib (CELEBREX®) and valdecoxib (BEXTRA™). A compound of this invention in combination with a cyclooxygenase-2 selective inhibitor could be administered in unit dosage form or separately to a patient on low-dose aspirin therapy. Alternatively, the cyclooxygenase-2 inhibitor could be administered in unit dosage form with low-dose aspirin, in which case a compound of this invention would be administered separately. All three active ingredients in unit dosage form is also encompassed. Conventional dosage amounts of the cyclooxygenase-2 selective inhibitor and aspirin (for cardio protection) may be utilized. For example, aspirin could be administered at 81 mg once daily.

In general, FLAP inhibitors can be identified as those compounds which have an IC₅₀ in the “FLAP Binding Assay” that is less than or equal to 1 μM, and preferably 500 nM or less.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk of onset of atherosclerosis.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

An effective amount of a FLAP inhibitor in the method of this invention is in the range of about 0.001 mg/kg to about 100 mg/kg of body weight per day, preferably 0.01 mg to about 10 mg per kg, and most preferably 0.1 to 1 mg per kg, in single or divided doses. A single daily dose is preferred but not necessary. On the other hand, it may be necessary to use dosages outside these limits in some cases. As examples, the daily dosage amount may be selected from, but not limited to 25 mg, 50 mg, 75 mg, 100 mg, 125 mg, 150 mg, 200 mg and 250 mg. It will be understood, however, that the specific dose level for any particular patient will depend upon a variety of factors including the age, body weight, general health, sex, diet, time of administration, route of administration, rate of excretion, drug combination and the severity of the patient's condition. A consideration of these factors is well within the purview of the ordinarily skilled clinician for the purpose of determining the therapeutically effective or prophylactically effective dosage amount needed to prevent, counter, or arrest the progress of the condition. It is expected that the FLAP inhibitor will administered chronically on a daily basis for a length of time appropriate to treat or prevent the medical condition relevant to the patient, including a course of therapy lasting months, years or the life of the patient.

In a broad embodiment, any suitable additional active agent or agents, including but not limited to anti-atherosclerotic agents, may be used in combination with the compound of Formula I in a single dosage formulation, or may be administered to the patient in a separate dosage formulation, which allows for concurrent or sequential administration of the active agents. One or more additional active agents may be administered with a compound of Formula I. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as ZD-4522, (CRESTOR®; see U.S. Pat. No. 5,260,440, and Drugs of the Future, 1999, 24(5), pp. 511-513); 5-lipoxygenase inhibitors; cholesterol ester transfer protein (CETP) inhibitors, for example JTT-705 and torcetrapib, also known as CP529,414; HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein MTP) inhibitors; niacin; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidinediones as well as those PPARγ agonists outside the thiazolidine dione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABCA1 gene expression; FXR and LXR ligands including both inhibitors and agonists; bisphosphonate compounds such as alendronate sodium; and cyclooxygenase-2 inhibitors such as celecoxib.

Still another type of agent that can be used in combination with the compounds of this invention are cholesterol absorption inhibitors. Cholesterol absorption inhibitors block the movement of cholesterol from the intestinal lumen into enterocytes of the small intestinal wall. This blockade is their primary mode of action in reducing serum cholesterol levels. These compounds are distinct from compounds which reduce serum cholesterol levels primarily by mechanisms of action such as acyl coenzyme A-cholesterol acyl transferase (ACAT) inhibition, inhibition of triglyceride synthesis, MTP inhibition, bile acid sequestration, and transcription modulation such as agonists or antagonists of nuclear hormones. Cholesterol absorption inhibitors are described in U.S. Pat. No. 5,846,966, U.S. Pat. No. 5,631,365, U.S. Pat. No. 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, WO 00/63703, WO/0060107, WO 00/38725, WO 00/34240, WO 00/20623, WO 97/45406, WO 97/16424, WO 97/16455, and WO 95/08532.

An exemplary cholesterol absorption inhibitor is ezetimibe, also known as SCH-58235, which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966 and shown below as

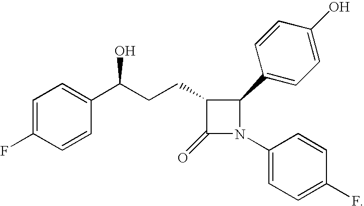

Additional exemplary hydroxy-substituted azetidinone cholesterol absorption inhibitors are specifically described in U.S. Pat. No. 5,767,115, column 39, lines 54-61 and column 40, lines 1-51, represented by the formula

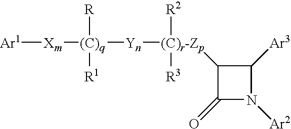

as defined in column 2, lines 20-63. These and other cholesterol absorption inhibitors can be identified according to the assay of hypolipidemic compounds using the hyperlipidemic hamster described in U.S. Pat. No. 5,767,115, column 19, lines 47-65, in which hamsters are fed a controlled cholesterol diet and dosed with test compounds for seven days. Plasma lipid analysis is conducted and data is reported as percent reduction of lipid versus control.

Therapeutically effective amounts of cholesterol absorption inhibitors include dosages of from about 0.01 mg/kg to about 30 mg/kg of body weight per day, preferably about 0.1 mg/kg to about 15 mg/kg. For an average body weight of 70 kg, the dosage level is therefore from about 0.7 mg to about 2100 mg of drug per day, e.g. 10, 20, 40, 100 or 200 mg per day, preferably given as a single daily dose or in divided doses two to six times a day, or in sustained release form. This dosage regimen may be adjusted to provide the optimal therapeutic response when the cholesterol absorption inhibitor is used in combination with a compound of the instant invention.

In the method of treatment of this invention, the FLAP inhibitors may be administered via any suitable route of administration such as orally, parenterally, or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. Oral formulations are preferred.

For oral use, the pharmaceutical compositions of this invention containing the active ingredient may be in forms such as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc.

Oral immediate-release and time-controlled release dosage forms may be employed, as well as enterically coated oral dosage forms. Tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. One example of a time-controlled release device is described in U.S. Pat. No. 5,366,738. They may also be coated by the technique described in U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds useful in the method of treatment of the invention may also be administered in the form of a suppository for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

A therapeutically effective amount of a compound of Formula I can be used for the preparation of a medicament useful for treating or preventing any of the medical conditions described herein, in dosage amounts described herein. For example, a compound of Formula I can be used for the preparation of a medicament useful for the treatment of asthma, allergies and allergic conditions, inflammation, COPD or erosive gastritis. Additionally, the medicament may be useful for preventing or reducing the risk of developing atherosclerotic disease, halting or slowing the progression of atherosclerotic disease once it has become clinically manifest, and preventing or reducing the risk of a first or subsequent occurrence of an atherosclerotic disease event. The medicament comprised of a compound of Formula I may also be prepared with one or more additional active agents, such as those described herein.

The compounds of structural Formula I of the present invention can be prepared according to the procedures of the following Schemes and Examples, using appropriate materials and are further exemplified by the specific examples which follow. Moreover, by utilizing the procedures described herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously hereinabove. The free amine bases corresponding to the isolated salts can be generated by neutralization with a suitable base, such as aqueous sodium hydrogencarbonate, sodium carbonate, sodium hydroxide, or potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation, or crystallization. All temperatures are degrees Celsius unless otherwise noted. Mass spectra (MS) were measured by electron-spray ion-mass spectroscopy.

The phrase “standard peptide coupling reaction conditions” means coupling a carboxylic acid with an amine using an acid activating agent such as HATU, EDC, and PyBOP in an inert solvent such as dichloromethane or DMF in the presence of a auxiliary nucleophile such as HOAT or HOBT. The use of protecting groups for the amine and carboxylic acid functionalities to facilitate the desired reaction and minimize undesired reactions is well documented. Conditions required to add and remove protecting groups are found in standard textbooks such as Greene, T, and Wuts, P. G. M., Protective Groups in Organic Synthesis, John Wiley & Sons, Inc., New York, N.Y., 1999. CBZ and BOC are commonly used amino protecting groups in organic synthesis, and their removal conditions are known to those skilled in the art. For example, CBZ may be removed by catalytic hydrogenation in the presence of a noble metal or its oxide such as palladium on activated carbon in a protic solvent such as methanol or ethanol. In cases where catalytic hydrogenation is contraindicated due to the presence of other potentially reactive functionalities, removal of CBZ groups can also be achieved by treatment with a solution of hydrogen bromide in acetic acid or by treatment with a mixture of TFA and dimethylsulfide. Removal of BOC protecting groups is carried out with a strong acid, such as trifluoroacetic acid, hydrochloric acid, or hydrogen chloride gas, in a solvent such as methylene chloride, dioxane, methanol, or ethyl acetate.

Some abbreviations used herein are as follows:

Ac is acetyl; aq. is aqueous; Ar is Aryl; 9-BBN is 9-Borabicyclo[3.3.1]nonane; BOC (Boc) is tert-butyloxycarbonyl; Bn is benzyl; Bu is butyl; celite is Celite® diatomaceous earth; CBZ (Cbz) is benzyloxycarbonyl; DCM is dichloromethane; DEAD is diethyl azodicarboxylate; Dess-Martin Periodinane is 1,1,1-tris(acetyloxy)-1,1-dihydro-1,2-benzodoxol-3-(1H)-one; DIAD is diisopropylazodicarboxylate; DIBAL-His diisobutylaluminum hydride; DIPEA is Diisopropylethylamine; DMAP is 4-dimethylaminopyridine; DMF is N,N-dimethylformamide; dppf is 1,1′-bis(diphenylphosphino)ferrocene; EDC is 1-(3-dimethylaminopropyl)-3-ethylcarbodiimide.HCl; equiv. is equivalent(s); ES is electron spray ion-mass spectroscopy; Et is ethyl; EtOAc is ethyl acetate; EtOH is ethanol; HATU is O-(7-azabenzotriazol-1-yl)-1,1,3,3-tetramethyluronium hexafluorophosphate; HCl is hydrogen chloride; HAR is heteroaryl; HOAt is 1-hydroxy-7-azabenzotriazole; HOBt is 1-hydroxybenzotriazole hydrate; HPLC is high performance liquid chromatography; i is iso; LDA is lithium diisopropylamide; LG is leaving group; m is meta; Me is methyl; MeOH is methanol; m.p. is melting point; MS is mass spectrum; Ms is methanesulfonyl; NMM is N-methylmorpholine; NMO is N-methylmorpholine-N-oxide; NMP is N-methylpyrrolidine; NMR is nuclear magnetic resonance; nOe is nuclear Overhauser effect; o is ortho; OAc is acetoxy, p is para, PCC is pyridinium chlorochromate; Ph is phenyl; Pr is propyl; p-TSA is para-toluenesulfonic acid; PyBOP is benzotriazol-1-yloxytripyrrolidinephosphonium hexafluorophosphate; R^{o}, R^{p}, R^{r}, R^{s}, R^{t}, R^{u}, R^{v}, R^{w}, R^{x}, R^{y} and R^{z} are unspecified substituents such that the definition of Formula I of the present invention is satisfied; sat. is saturated; SFC is supercritical fluid chromatography; t is tert; ^{t}Bu is tert-butyl; Tf is trifluoromethanesulfonyl; TFA is trifluoroacetic acid; THF is tetrahydrofuran; TLC is thin layer chromatography; and TPAP is tetrapropylammonium perruthenate.

Reaction schemes A-Q illustrate the methods employed in the synthesis of the compounds of the present invention of structural Formula I. All abbreviations are as defined above unless indicated otherwise.

Reaction scheme A illustrates the preferred method of synthesis of a compound of type 8 (a≧1). In this method, a hydroquinone derivative of type 1 is treated with an acyl halide of type 2 in an electrophilic aromatic substitution process generally referred to as the Friedel-Crafts acylation reaction. The reaction is usually conducted in the presence of a Lewis acid like aluminium trichloride, or boron trifluoride or the like, but can also be catalyzed with graphite. It is customary to conduct the reaction in an inert organic solvent like benzene, or toluene, at temperatures between room temperature and the boiling point of the solvent. The resulting ketone 3 is then subjected to a Pinacol coupling with a second ketone of type 4 to afford an unsymmetrical diol of type 5. The Pinacol coupling can be promoted with a number of active metals such as sodium, magnesium or aluminum, and more recently, low valent titanium. Low valent titanium (LVT) is particularly reactive and can be prepared from the reduction of titanium tetrachloride or titanium trichloride with reducing agents such as sodium, magnesium, zinc, zinc-copper couple, aluminum or the like. In order to avoid extensive self coupling of either carbonyl component, it is typical to conduct the reaction with an excess of one of the coupling partners. When the reaction is conducted with LVT, it is customary to employ an ethereal solvent like diethyl ether, or THF or the like, at temperatures between room temperature and the boiling point of the solvent, for 6-48 hours. The product diol 5 is dehydrated to ketone 6 via the well known Pinacol-Pinacolone rearrangement. The classical conditions for performing such a transformation involve the use of a strong Brönsted acid such as sulfuric acid or the like, or alternatively, a weaker Brönsted acid like acetic acid in conjunction with catalytic amounts of iodine may also be used to effect this transformation. Removal of the carbonyl functionality of 6 can be achieved using a variety of methods known in the chemical literature, such as the Wolff-Kishner reduction. In this method, hydrazine hydrate is allowed to react with 6, in the presence of base, typically potassium hydroxide, at elevated temperatures up to 200° C., in a solvent such as diethylene glycol. Demethylation of 7 is achieved with a reagent such as boron tribromide, or bromodimethylborane or the like, in an inert organic solvent like DCM, or 1,2-dichloroethane, and the product of the reaction is a dihydroxyphenyl derivative of type 8, which can be elaborated to compounds of the present invention as described in the subsequent schemes.

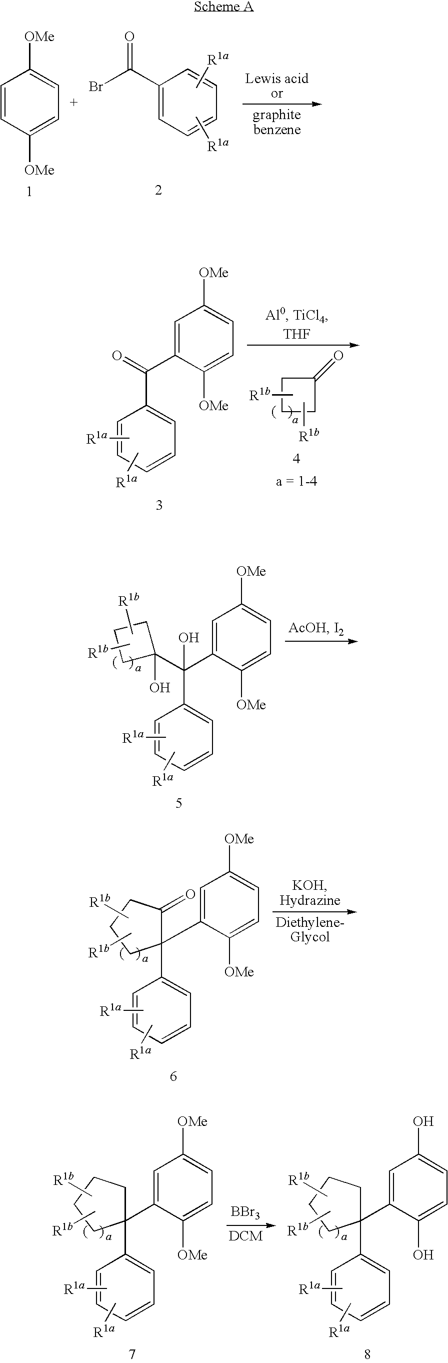

Reaction scheme B illustrates an alternative method for generating compounds of structural formula 15. In this method, an acetophenone of type 9 is treated with an organometallic reagent of type 10, capable of transferring an aryl group. Preferred organometallic reagents for this transformation include organomagnesium (Grignard) or organolithium compounds. When Grignard reagents are employed as shown in scheme B, it is customary to conduct the reaction in a suitable ethereal solvent such as diethyl ether, or THF or mixtures thereof, at temperatures between −78° C. and the boiling temperature of the solvent. In the case of an organolithium reagent, the reaction can be conducted in a variety of solvents such as diethyl ether or hexanes, at temperatures between −78° C. and room temperature. The Grignard and the organolithium reagents are often purchased commercially, but can be prepared synthetically according to known methods in organic synthesis. The resulting alcohol 9a can be dehydrated to an olefin of type 11 in the presence of a suitable protic acid such as p-TSA or the like. The reaction is usually conducted in an organic solvent like MeOH, or benzene, or the like, at temperatures between room temperature and the boiling point of the solvent, for 1-12 hours. Olefin 11 can then be converted to a cyclobutanone of type 12 in a [2+2]cycloaddition process involving ketene or a ketene equivalent. Since ketene is a highly poisonous gas, it is generally more convenient to use a ketene equivalent generated in situ, Convenient methods for the generation of ketenes include dehydrohalogenation of acyl chlorides or dehalogenation of α-halo acyl chlorides. Accordingly, sonication of trichloroacetyl chloride with zinc dust generates dichloroketene which participates in a [2+2]cycloaddition reaction with 11 to afford the cycloaddition product 12. The reaction is usually conducted in an ethereal solvent like diethyl ether, or THF, at room temperature, for 12-24 hours. Dehalogenation of 12 can be achieved in the presence of zinc dust and a mild protic acid such as acetic acid, at temperatures between 50-100° C., for 6-12 hours. The resulting ketone 13, which is formally a cycloaddition product between 11 and ketene, is then transformed to 15, following the procedures described in the discussion for Scheme A, which can then be elaborated to compounds of the present invention as described in the subsequent schemes.

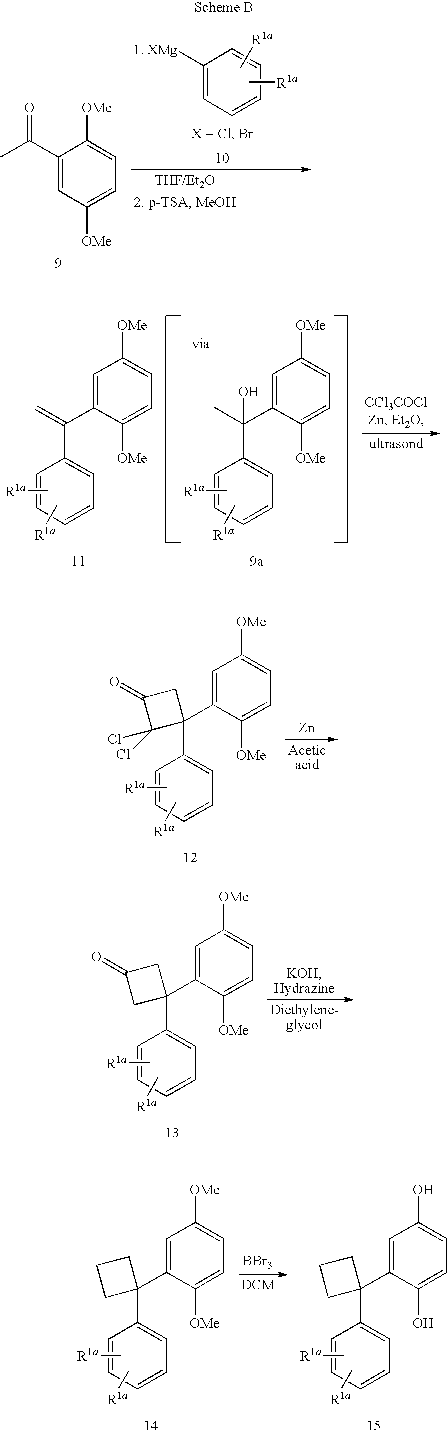

Reaction scheme C illustrates a preferred strategy for the conversion of compounds of type 13 to compounds of structural formula 16, 17 and 18. In this method, a single ring homologation of cyclobutanone 13 affords a cyclopentanone of type 16, which after a second subsequent ring homologation, furnishes a mixture of regiosomeric cyclohexanones of type 17 and type 18. Preferred conditions for effecting the ring expansion include the method of Yamamoto (K. Maruoka, A. B. Concepcion and H. Yamamoto, Synthesis 1994, 1283-1290) in which the ketone derivative is treated with diazomethane in the presence of an organoaluminum reagent such as trimethylaluminum or methyl aluminum bis(2,6-di-tert-butyl-4-methylphenoxide) (MAD) or the like. The reaction is usually conducted in an inert organic solvent like DCM, and at low temperature, preferably −78° C., for periods of 1-3 hours. Reduction of 16 according to the aforementioned Wolff-Kishner method affords 7 (a=1) while analogous reduction of either/both 17 and 18 affords 7 (a=2).

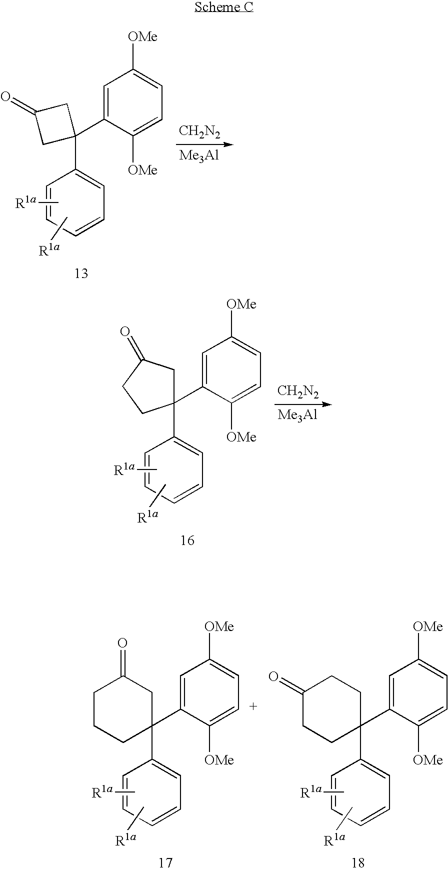

Reaction scheme D illustrates a preferred method of synthesis of compounds of structural formula 20. In this method, an olefin of type 11 can be transformed to a cyclopropane of type 19, in the presence of carbene or a suitable carbenoid. Convenient methods for the generation of a carbenoid species include the treatment of dihalogenated precursors like diiodomethane, or chloroiodomethane, or the like with zinc/copper couple, or a dialkyzinc reagent. The resulting zinc carbenoid adds to 11 to form cyclopropane 19. It is customary to conduct the reaction in an ethereal solvent like diethyl ether, or t-butylmethyl ether or the like, at temperatures between room temperature and boiling point of the solvent, for 12-24 hours. Demethylation of 19 is achieved according to the conditions described in the discussion for scheme A, and the resulting dihydroxyphenyl derivative 20 can be elaborated to compounds of the present invention as shown in the subsequent schemes.

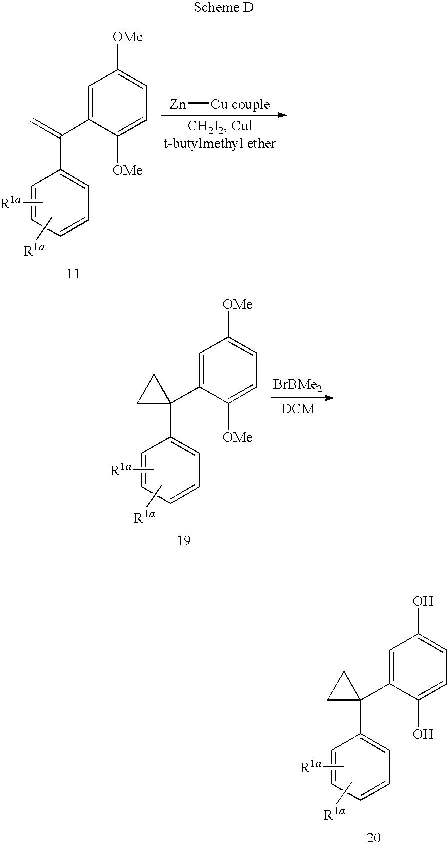

Reaction scheme E illustrates the synthesis of a compound of structural formula 23 in which it is desirable to first elaborate the more reactive hydroxyl group (1-position) of 21. For example, 21 can be directly alkylated using an alkylating agent of type 22. The reaction is conducted typically in the presence of a suitable base such as potassium carbonate or cesium carbonate, in a polar aprotic solvent such as DMF, in which the substituent LG of 22 is a good leaving group such as halide, mesylate or triflate. The major products from the reaction are the mono-allylated product of structural formula 23 and the bis-alkylated product of structural formula 25 which can be readily separated by flash chromatography. In some cases, a small amount of the regioisomeric mono-alkylated product 24 is observed.

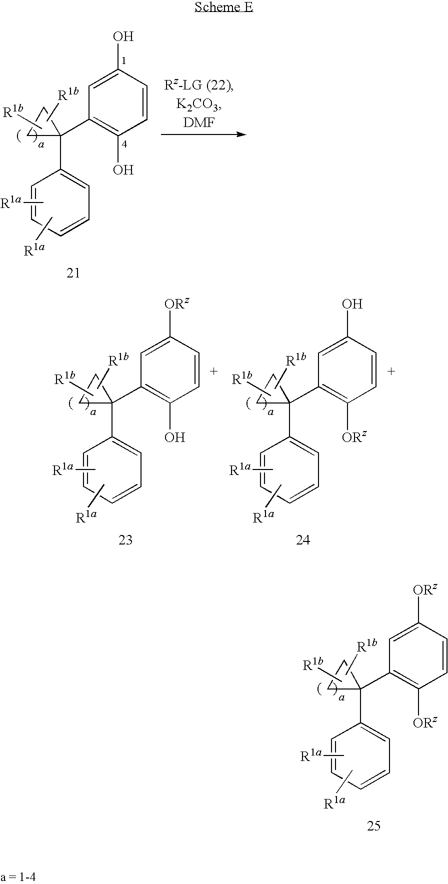

Reaction scheme F illustrates a protecting group strategy for the synthesis of a compound of type 29 in which it is desirable to elaborate the less reactive hydroxyl group (4-position) of 21. For example, the more reactive hydroxyl group (1-position) in 21 can be selectively protected with a range of groups known in organic synthesis, exemplified in this case by a silicon-based protecting group approach. In this method, 21 is treated with a suitable silylating agent such as chloro-tert-butyldiphenylsilane, in the presence of imidazole, in a solvent like DMF. The reaction is conducted typically at temperatures between 0° C. and room temperature, for periods of 12-24 hours. The product is a silyl ether of type 26, which can be directly alkylated using the conditions described in the discussion for scheme E to afford a product of type 28. The silicon protecting group can be removed by any of the appropriate desilylation methods such as treatment with TBAF in THF, or hydrogen fluoride in pyridine, and the product of this reaction is a phenol of type 29.

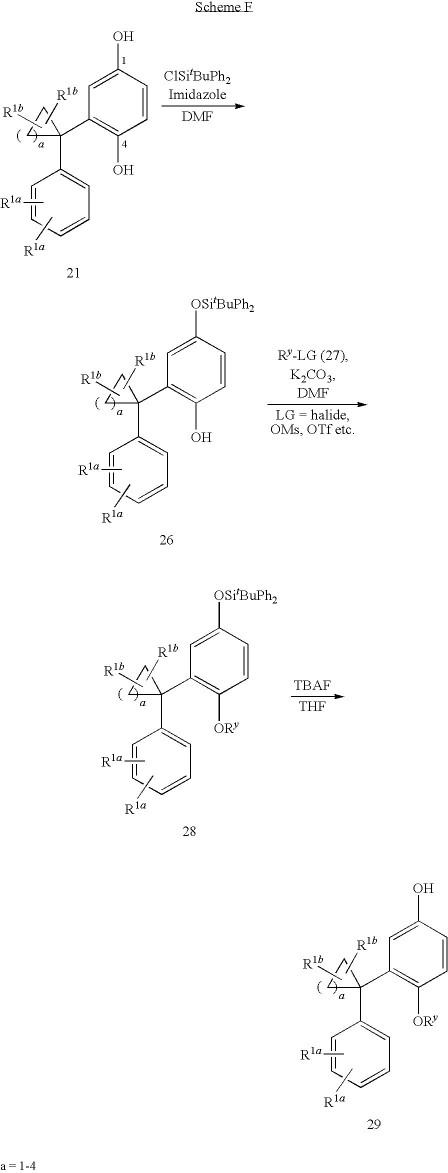

Reaction scheme G illustrates some of the preferred methods for the elaboration of 23. For example, 23 can be treated with a triflating agent such as triflic anhydride or the like in the presence of a suitable base such as pyridine or triethylamine in an aprotic solvent like toluene. It is customary to conduct the reaction at temperatures between −78° C. and room temperature, for periods of 1-24 hours. The product of the reaction is a triflate of structural formula 30 which can be elaborated by a variety of synthetic methods known to those skilled in organic synthesis, three of which are outlined in schemes H I, and J.

Alternatively, 23 can be treated with an isocyanate of type 31 in the presence of a suitable base such as triethylamine, in an inert solvent like toluene (scheme F). Typically, the isocyanate reagent 31 can be purchased commercially or prepared synthetically, and the product of the reaction is a carbamate of type 32, In certain cases it may be preferable to generate 31 in situ, and this is typically accomplished from an appropriate precursor such as an acyl azide. In an alternative method, 23 can be treated with a suitable carbonyl equivalent such as phosgene, or triphosgene or carbonyl diimidazole. After a short period of time, typically between 0.1-1 hour, a primary or secondary amine is added and the product of the reaction is a carbamate of structural formula 32. The reaction sequence is conducted in a suitable inert organic solvent like DCM, at temperatures between 0° C. and room temperature, for periods of 1-24 hours.

In yet another example, 23 can be directly alkylated using the conditions described in the discussion of scheme D to afford a derivative of type 34.

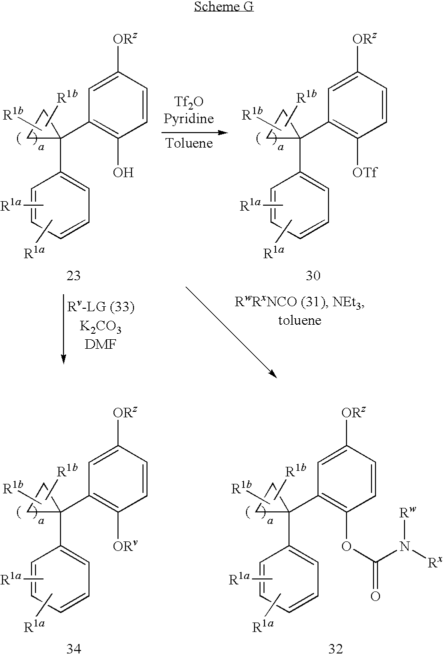

Reaction scheme H illustrates the preferred method of synthesis of compounds of structural formula 35, 36 and 37. In this method, 30 is treated with either allyltributylstannane or vinyltributylstannane in the presence of a suitable a palladium catalyst such as [1,1′-bis(diphenylphosphino)-ferrocene]dichloropalladium(II), in an inert organic solvent like DMF or NMP. The reaction is usually conducted at elevated temperatures, typically between 50-120° C., for periods of 2-24 hours. In certain cases, it may be essential to use an additive such as lithium chloride to promote the reaction. Often, the reaction times can be significantly reduced if the reaction is conducted under microwave irradiation. The product of the reaction is an alkene of structural formula 35, which can be synthetically elaborated, using a variety of methods known in organic synthesis. For example, 35 can be oxidatively cleaved to afford an aldehyde of type 36, which can be further oxidized to a carboxylic acid derivative of structural formula 37. A preferred method for the oxidative cleavage reaction is the two-step process shown in reaction scheme H. Alkene 35 is first oxidized to a vicinal diol using catalytic osmium tetraoxide in the presence of a stoichiometric reoxidant such as NMO, in a solvent system such as acetone-water. The intermediate vicinal diol which forms is generally not isolated, but is in turn subjected to cleavage with sodium periodate in a suitable mixed solvent system like THF-water to afford 36. Both steps in the oxidative cleavage sequence are generally completed during periods of several minutes to a few hours, at temperatures between 0° C. and room temperature. Alternatively, the oxidative cleavage of 35 may also be accomplished using ozone, or by other methods known to those skilled in the art. Aldehyde 36 can then be further oxidized to 37 using a buffered chlorite oxidation system. In this method, 36 is treated with sodium chlorite and monobasic sodium phosphate in the presence of a chlorine scavenger, such as 2-methyl-2-butene. The reaction is conducted typically in a solvent system like n-butanol-water, for periods of 1-6 hours, at temperatures between 0° C. and room temperature. In certain cases, 35 can be directly converted to 37 using the sodium periodate/ruthenium trichloride reagent system. Both 36 and 37 can be elaborated in numerous ways known in organic synthesis to furnish other compounds of the present invention.

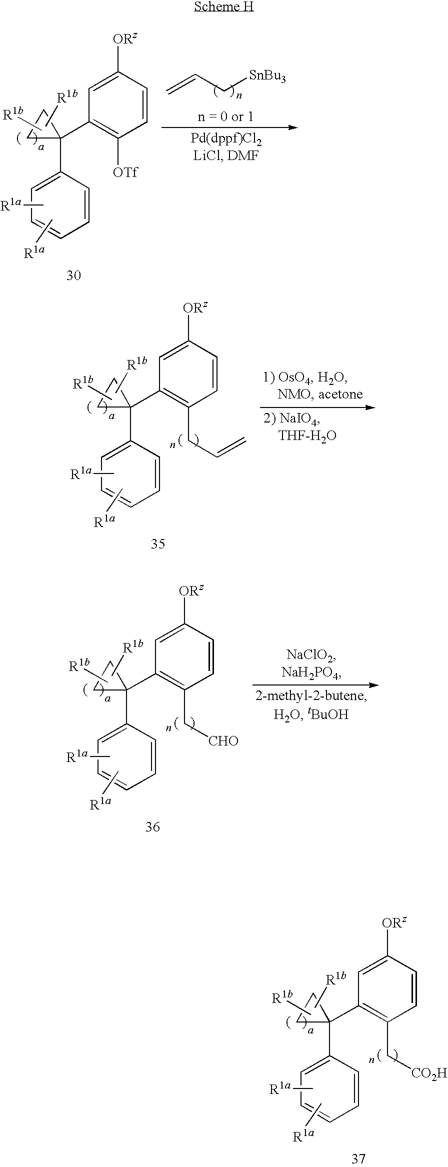

Reaction scheme I illustrates an alternative method of synthesis of a compound of structural formula 37 (n=0). In this method, 30 is treated with MeOH in the presence of a suitable palladium catalyst such as [1,1′-bis(diphenylphosphino)ferrocene]dichloropalladium(II), in an inert organic solvent like DMF. The reaction is usually conducted at elevated temperature, typically between 50-100° C., for periods of 6-24 hours, under an atmosphere of carbon monoxide. In certain cases it may be preferable to use elevated pressures of carbon monoxide or an additive such as lithium chloride to promote or accelerate the reaction. In certain cases, it may be preferable to perform the reaction under the influence of microwave irradiation. The product of the reaction is an ester of structural formula 38 which can be converted to 37 (n=0) using a variety of hydrolytic methods known to those skilled in organic synthesis. A compound of type 30 can also be converted to a compound of structural formula 39, again using organopalladium based methods. For example, 30 can be treated with a cyanide source, such as zinc cyanide, or potassium cyanide or the like, in the presence of a suitable palladium catalyst/ligand reagent system. It is customary to conduct the reaction in inert organic solvent, preferably a dipolar aprotic solvent such as DMF, or NMP or the like, at elevated reaction temperatures, typically between 50-140° C., for periods of 6-24 hours. The product of the reaction is a nitrile derivative of type 39, which like 33 and 32, can be elaborated to other compounds of the present invention.

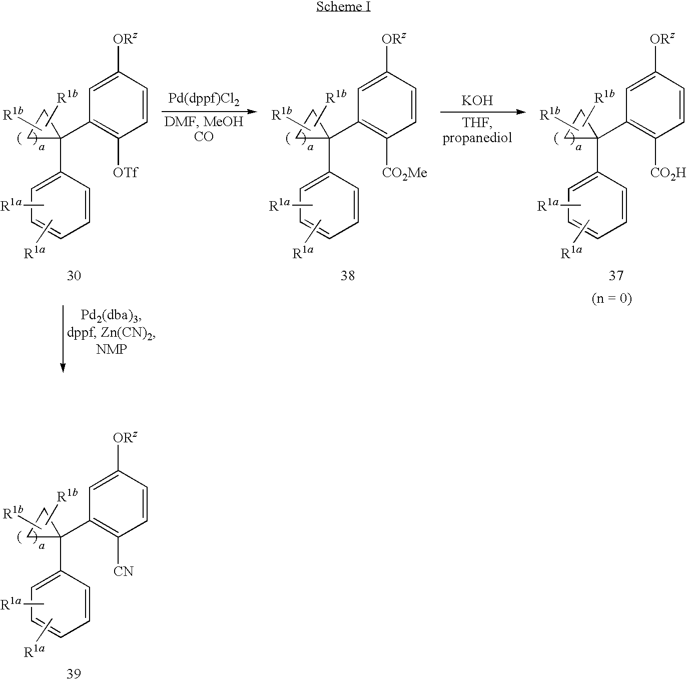

Reaction scheme J illustrates the preferred method of synthesis of a compound of structural formula 41. In this method, commonly referred to as the Suzuki reaction, 30 is treated with an aryl- or heteroaryl-boronic acid of type 40 in the presence of a suitable palladium catalyst such as [1,1′-bis(diphenylphosphino)ferrocene]-dichloropalladium(II) and aqueous sodium carbonate. The reaction is usually performed in a suitable combination of inert organic solvents such as toluene-EtOH, at about 80° C., for a period of 6-24 hours, and the product is a (hetero)biaryl of structural formula 41.

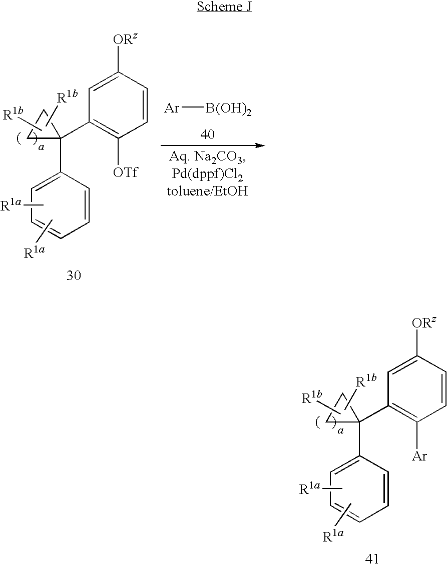

Reaction Scheme K illustrates the synthetic methodology in the most general case in which 37 is treated with an amine of type 42 to afford an amide of type 43. The amide bond coupling reaction illustrated in reaction scheme J is conducted in an appropriate inert solvent such as DMF, DCM or the like and may be performed with a variety of reagents suitable for amide coupling reactions such as HATU, EDC or PyBOP. Preferred conditions for the amide bond coupling reaction shown in reaction Scheme J are known to those skilled in organic synthesis. Such modifications may include, but are not limited to, the use of basic reagents such as triethylamine, DIPEA, or NMM, or the addition of an additive such as HOAt or HOBt. Alternatively, 42 may be treated with an activated ester or acid chloride derivative of 37, which also affords 43. The amide bond coupling shown in reaction Scheme J is usually conducted at temperatures between 0° C. and room temperature, occasionally at elevated temperatures, and the coupling reaction is typically conducted for periods of 1 to 24 hours.

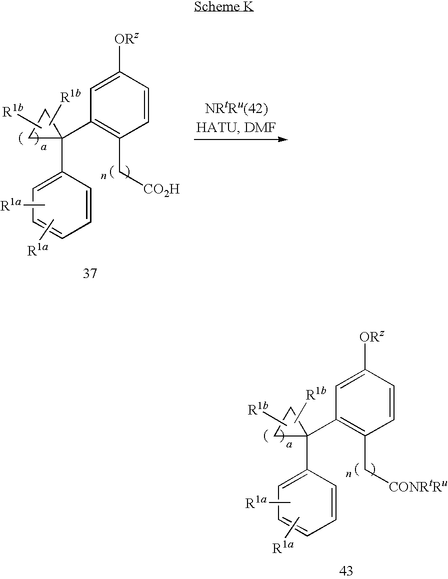

Reaction Scheme L illustrates a preferred method for the synthesis of a compound of type 45. In this method, 37 is subjected to the Curtius reaction to afford the N-Boc protected amine of structural formula 44. The reaction is performed by reacting 37 with diphenylphosphoryl azide in the presence of a tertiary amine such as triethylamine or DIPEA in a solvent such as toluene. The initial product is generally accepted to be the acyl azide, which is rearranged to the isocyanate in a thermal process analogous to the Wolff rearrangement of acyl carbenes. The rearrangement is conducted typically at the reflux temperature of the solvent, for instance 110° C., and the rearrangement is usually completed in periods of 1-5 hours. The intermediate isocyanate which forms is generally not isolated, but is in turn subjected to in situ reaction with a suitable alcohol such as tert-butyl alcohol to afford carbamate 44. The N-Boc group can be removed by a suitable deprotection method such as treatment with hydrogen chloride in EtOAc or TFA in DCM. The deprotection is conducted typically at temperatures between 0° C. and room temperature, and the reaction is usually complete in 0.5-3 hours. The product amine of structural formula 45 can be used as a coupling partner in reaction Scheme M or synthetically modified using a variety of methods known in organic synthesis to afford compounds of the present invention.

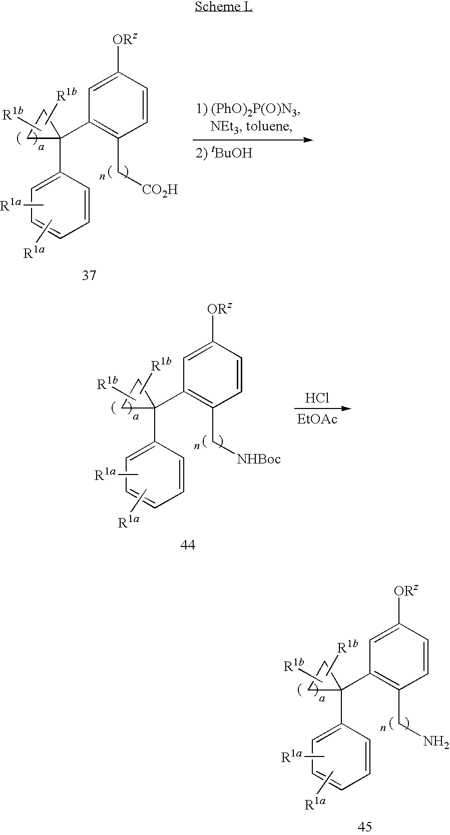

Reaction scheme M illustrates preferred methods for the syntheses of compounds of type 48. For example, 45 can participate in amide bond coupling reactions with a carboxylic acid of type 46 to afford an amide structural formula 48, using the reagents and conditions described for the generalized amide coupling protocol shown in reaction Scheme K. Alternatively, 45 may also be treated with an activated ester or acid chloride derivative of type 47, which also affords 48. Typical conditions for effecting such a transformation include treatment of 45 with acid chloride 47 in the presence of a tertiary amine base such as triethylamine. It is customary to perform the reaction in an inert organic solvent such as DMF or DCM, at temperatures between 0° C. and the reflux temperature of the solvent, frequently at room temperature and for periods of 1-24 hours.

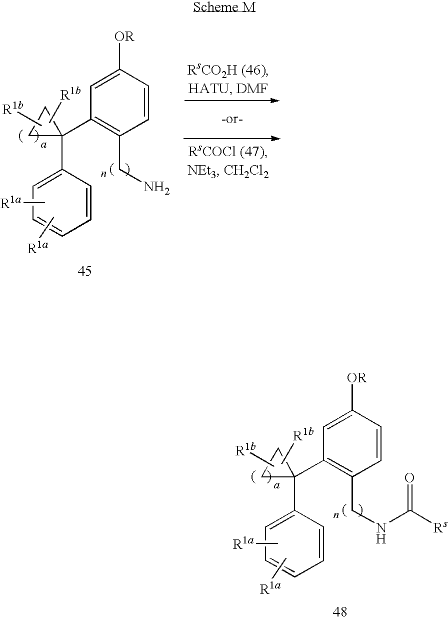

As shown in reaction scheme N, 45 can also be elaborated using the Fukuyama modification of the Mitsunobu reaction (Fukuyama, T.; Jow, C.-K.; Cheung, M. Tetrahedroin Lett. 1995, 36, 6373-74). For example, 45 may be reacted with an arylsulfonyl chloride such as 2-nitrobenzenesulfonyl chloride, 4-nitrobenzenesulfonyl chloride or 2,4-dinitrobenzenesulfonyl chloride and a tertiary amine base such as 2,4,6-collidine or 2,6-lutidine in an inert organic solvent such as DCM. Alternatively, the reaction can also be performed under the classical Schotten-Baumann conditions as shown in scheme M, in which 45 and the arylsulfonyl chloride are allowed to react in aqueous alkaline solution. The product of this reaction is a sulfonamide of type 49, which can be further modified by reaction with an alcohol of type 50 in the presence of triphenylphosphine and an activating agent such as DEAD, DIAD, or the like. The reaction is performed in a suitable inert organic solvent such as benzene, toluene, THF or mixtures thereof, typically at room temperature, and the reaction is generally complete in 0.5-3 hours. The product of this reaction is a sulfonamide of type 51, which can be desulfonylated in the presence of either a nucleophilic amine like ii-propylamine, in a solvent such as DCM, or with a combination of mercaptoacetic acid and triethylamine in DCM. In either case, the reaction is conducted typically at room temperature, for periods of 5 minutes to 1 hour. When a 2- or 4-nitrobenzenesulfonyl derivative is employed, the cleavage of the sulfonamide is accomplished with either combinations of thiophenol and potassium carbonate in a solvent like DMF, or with mercaptoacetic acid and lithium hydroxide in DMF. In either case, the reaction is conducted at room temperature, for periods of 1-3 hours. The secondary amine product of type 52 can be modified further using a variety of methods known in organic synthesis to provide other compounds of the present invention. For example, 52 may be subjected to a reductive amination reaction with an aldehyde or ketone of type 53 to afford compounds of type 55. Typical conditions for effecting such a reductive amination include preforming an imine 54 from aldehyde/ketone 53 and amine 52 followed by reduction of the intermediate imine with reagents capable of reducing carbon-nitrogen double bonds such as sodium borohydride, sodium cyanoborohydride or the like. Formation of the intermediate imine 54 may occur spontaneously in solution or it may be promoted with Lewis acid type reagents such as titanium (IV) isopropoxide or magnesium sulfate or the like. The formation of the imine is generally performed at temperatures between 0° C. and the reflux temperature of the solvent, frequently at room temperature. The imine formation step is generally allowed to proceed to completion over a period of several hours to 1 day prior to the reduction step which minimizes the formation of alcohol byproducts formed by simple reduction of the keto group in compounds of general formula 53. The intermediate imine 54 may in some cases be isolated and purified, however it is generally preferred to use it directly in the reduction step. The reduction of the imine 54 is typically conducted in an alcohol based solvent such as MeOH or EtOH at temperatures between 0° C. and room temperature, and the reduction is generally completed in a period of several hours or less.

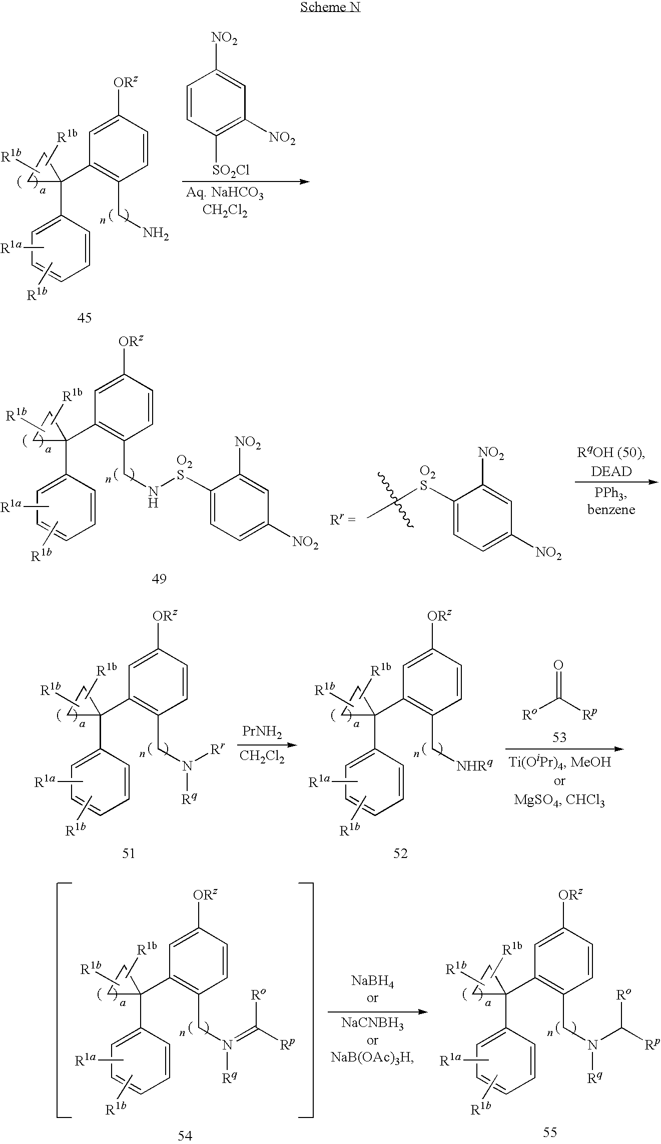

Reaction scheme O illustrates the preferred method of synthesis of compounds of structural formula 60 and 61, in which group X (X—CR²R³—Y) of the present invention is a carbon atom. In this method, 56 is initially converted to triflate 57 using either the conditions described in scheme G, or variations thereof. Cross-coupling of 57 with a terminal alkyne of type 58, in the presence of a suitable palladium catalyst, is referred to as the Sonogashira reaction. In the latter process, a copper(I) salt such as copper(I) iodide is also employed as co-catalyst, and the reaction is typically performed in the presence of an excess of amine base such as triethylamine, or diethylamine or the like. The reaction is conducted in an inert organic solvent such as DMF, at temperatures ranging from ambient temperature to about 100° C., for a period of 6-24 hours. The product of the reaction is an alkyne of type 59 which can then be transmogrified to an alkene derivative of type 60 or a saturated alkane derivative of type 61. If 60 is desired, preferred conditions for performing the partial reduction of 59 involve the use of a Lindlar catalyst reagent system under an atmospheric or elevated pressure of hydrogen. The reaction is usually conducted in an inert organic solvent such as EtOH, or EtOAc, or combinations thereof, and at room temperature for a period of 3-15 hours. If 61 is desired, then the reduction of 59 is performed with any one of a variety of palladium-on-carbon catalysts, at either atmospheric or elevated pressure of hydrogen.

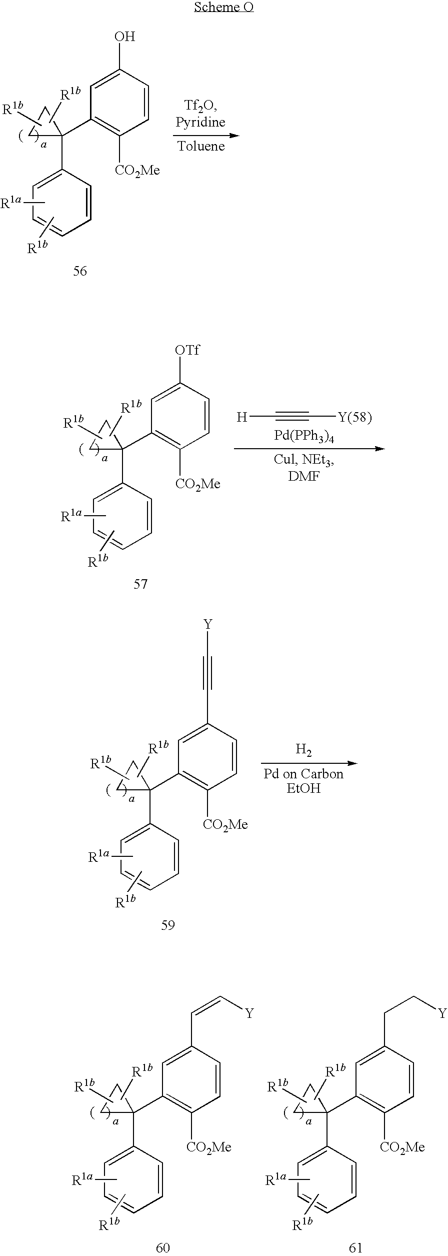

Scheme P illustrates that compounds of structural formula 62 can be elaborated to a variety of heterocyclic derivatives of structural formula 63 using methods known to those skilled in the art of organic synthesis. Specific examples of such transformations are shown in the Examples section. Leading references for effecting such transformations include:
1) Joule, J. A; Mills, K. and Smith, G. F. Heterocyclic Chemistry, Chapman & Hall, 1995, 3rd Edn., and references cited therein.
2) Katrittzky, A. R.; Rees, C. W. (Eds), Comprehensive Heterocyclic Chemistry: The Structure, Reactions, Synthesis, and Uses of Heterocyclic Compounds, Pergamon Press, Oxford, 1984, 8v, and references cited therein.
3) Comprehensive Heterocyclic Chemistry II: Review of the Literature 1982-1995: The Structure, Reactions, Synthesis and Uses of Heterocyclic Compounds, Pergamon Press, New York, 2nd Edn., 1996, 11v, and references cited therein.

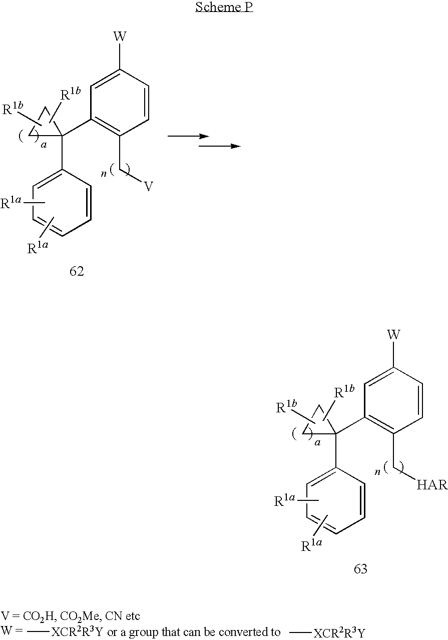

Scheme Q illustrates the preferred method for the resolution of a compound of structural formula 64 in which the asterisked carbon is a center of chirality. Generally, the latter, or intermediates en route to their preparation, may be resolved to afford enantiomerically pure compounds such as 65 and 66 by chiral stationary phase liquid chromatography techniques or other suitable methods known in organic synthesis.

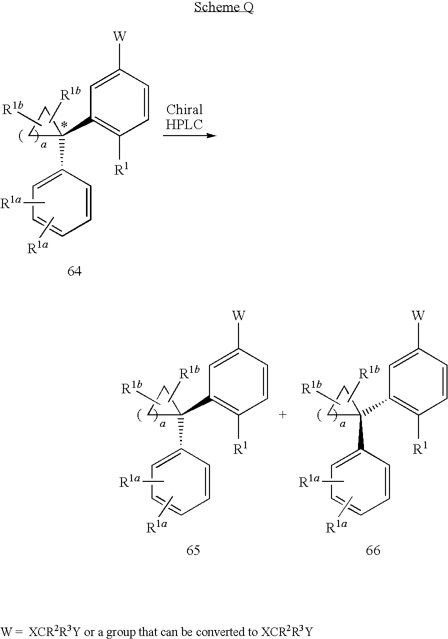

The following examples are provided to illustrate the invention and are not to be construed as limiting the scope of the invention in any manner.

### Preparation of Intermediates

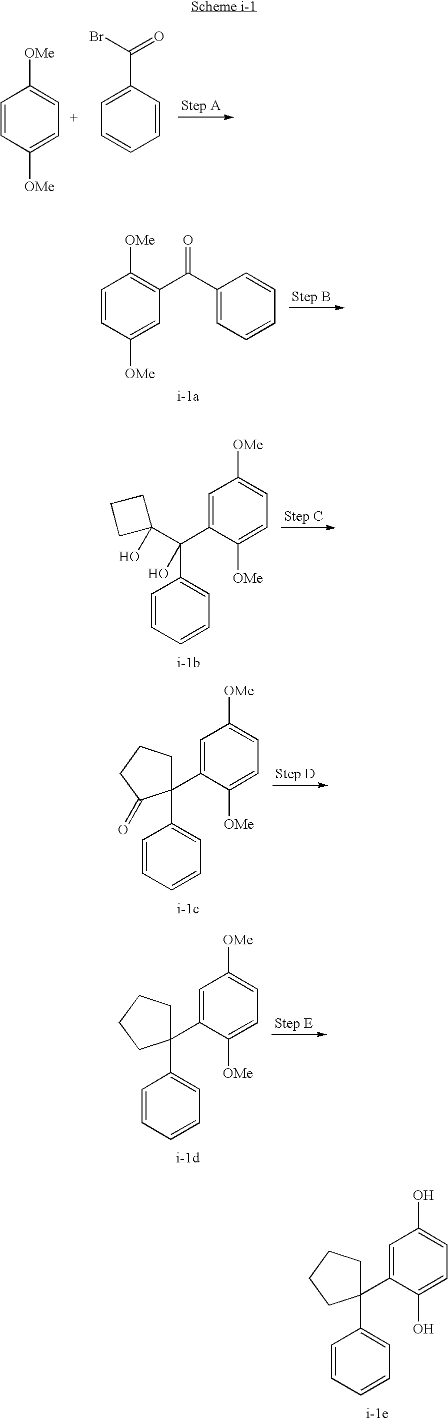

### 2-(1-phenylcyclopentyl)benzene-1,4-diol (1- e)

### Step A: Preparation of (2,5-dimethoxyphenyl)(phenyl)methanone (i-1a)

Benzoyl bromide (2.6 mL, 21.8 mmol) and graphite (1.0 g) were added to a stirred solution of 1,4-dimethoxybenzene (2.0 g, 14.5 mmol) in benzene (36 mL) at room temperature and the resulting mixture was heated to reflux for 8 h. After cooling to ambient temperature, the reaction mixture was filtered through a short plug of Celite®. The filtrate was washed with sat. aq. sodium bicarbonate, brine, dried (MgSO₄) and concentrated in vacuo. Purification of the crude residue by flash chromatography on silica gel (gradient elution; 0-25% EtOAc/hexanes as eluent) afforded the title compound 1-1a, together with the mono-desmethyl products (2-hydroxy-5-methoxyphenyl)(phenyl)methanone and (5-hydroxy-2-methoxyphenyl)(phenyl)methanone, which were used collectively in the next step.

### Step B: Preparation of Compound 1-[(2,5-dimethoxyphenyl)(hydroxy)phenylmethyl]cyclobutanol (i-1b)

Aluminum powder (1.28 g, 47.8 mmol) was added to a mixture of i-1a, (2-hydroxy-5-methoxyphenyl)(phenyl)methanone, and (5-hydroxy-2-methoxyphenyl)(phenyl)methanone (1.93 g), and cyclobutanone (1.19 mL, 15.9 mmol) in THF (100 mL) at room temperature. After cooling to approximately 0° C., titanium tetrachloride (3.42 mL, 31.9 mmol) was added via syringe and the resulting mixture heated to reflux for 90 min. After cooling to ambient temperature, the reaction mixture was stirred for a further 2 d. The reaction mixture was precipitated with ether, filtered and the filtrate concentrated in vacuo. The crude residue was resuspended in ether and washed with water, brine, dried (MgSO₄) and concentrated in vacuo. Purification of the crude residue by flash chromatography on silica gel (gradient elution; 0-20% EtOAc/hexanes as eluent) afforded the title compound i-1b.

### Step C: Preparation of 2-(2,5-dimethoxyphenyl)-2-phenylcyclopentanone (i-1c)

Iodine (a few crystals) was added to a solution of i-1b (315 mg, 1.0 mmol) in acetic acid (15 mL) at room temperature and the resulting solution heated at reflux for 1 h. After cooling to room temperature, the volatiles were evaporated in vacuo, the residue suspended in EtOAc, and washed with sat. aq. sodium bicarbonate, brine, dried (MgSO₄) and concentrated in vacuo. Purification of the crude residue by flash chromatography on silica gel (gradient elution; 0-25% EtOAc/hexanes as eluent) afforded the title compound i-1c.

### Step D: Preparation of 1,4-dimethoxy-2-(1-phenylcyclopentyl)benzene (1-Id)

Hydrazine mono-hydrate (624 μL, 12.8 mmol) was added to a solution of i-1c (173 mg, 0.58 mmol) in diethylene glycol (8.0 mL), and the resulting solution heated to 160° C. with the allowance for removal of volatiles. After 45 min, the reaction mixture was cooled to room temperature, potassium hydroxide (1.08 g, 19.1 mmol) was added and the resulting solution heated to 175-195° C. for 18 h. After cooling to room temperature, the reaction mixture was neutralized with 1 N hydrochloric acid and extracted three times with EtOAc. The combined organic extracts were washed with water, brine, dried (MgSO₄) and concentrated in vacuo. Purification of the crude residue by flash chromatography on silica gel (gradient elution; 0-25% EtOAc/hexanes as eluent) afforded the title compound i-1d, together with the mono-desmethyl derivatives: 4-methoxy-2-(1-phenylcyclopentyl)phenol, and 4-methoxy-3-(1-phenylcyclopentyl)phenol, which were used collectively in the next step.

### Step E: Preparation of 2-(1-phenylcyclopentyl)benzene-1,4-diol (i-1e)

Boron tribromide (333 μL of a 1 M solution in DCM) was added to a solution of i-1d, 4-methoxy-2-(1-phenylcyclopentyl)phenol, and 4-methoxy-3-(1-phenylcyclopentyl)phenol (94.0 mg) in DCM (3.33 mL) and the resulting solution allowed to stir at room temperature for 2 d. The reaction mixture was poured into ice-water and extracted twice with DCM. The combined organic extracts were washed with brine, dried (MgSO₄), filtered and concentrated in vacuo. Purification of the crude residue by flash chromatography on silica gel (gradient elution; 0-30% EtOAc/hexanes as eluent) afforded the title compound 1-1e. ¹HNMR (500 MHz, CDCl₃): δ 1.76-1.82 (m, 4H), 2.23-2.35 (m, 4H), 6.66 (d, J=6.6 Hz, 2H), 7.03 (t, J=1.6 Hz, 1H), 7.23-7.28 (m, 1H), 7.32-7.32 (m, 4H).

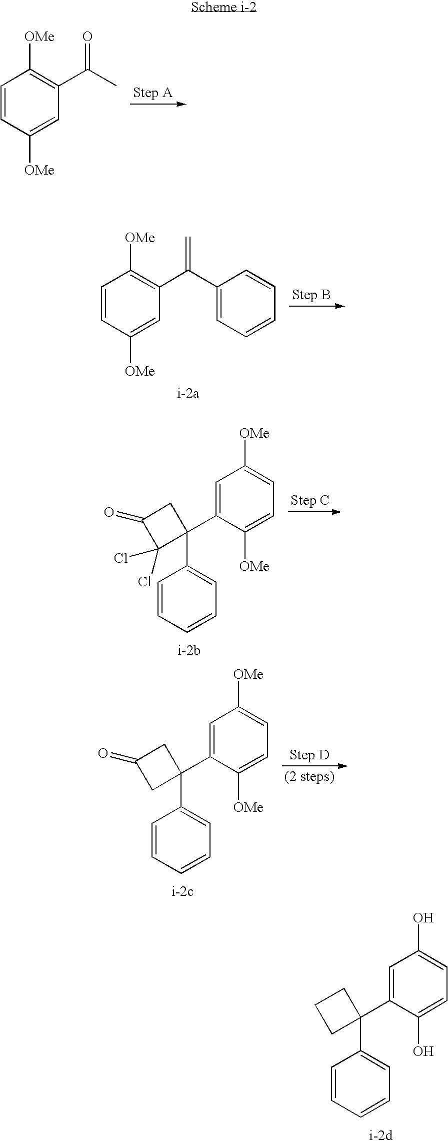

### Preparation of 2-(1-phenylcyclobutyl)benzene-1,4-diol (i-2d)

### Step A: Preparation of 1,4-dimethoxy-2-(1-phenylvinyl)benzene (i-2a)

2,5-Dimethoxyacetophenone (1.13 g, 6.27 mmol) was added dropwise to a solution of phenylmagnesium bromide (9.4 mL of a 1 M solution in THF, 9.4 mmol) in ether (30 mL) at 0° C. and the resulting solution stirred at 0° C. for 72 h. The reaction mixture was poured into sat. aq. ammonium chloride and extracted three times with EtOAc. The combined organic extracts were dried (MgSO₄) and concentrated in vacuo. The crude material was suspended in MeOH (30 mL), treated with p-TSA (300 mg), and heated at reflux for 1.5 h. After cooling to room temperature, the reaction mixture was poured into sat. aq. ammonium chloride and extracted three times with EtOAc. The combined organic extracts were dried (MgSO₄) and concentrated in vacuo. Purification of the crude residue by flash chromatography on silica gel (gradient elution; 0-20% EtOAc/hexanes as eluent) afforded the title compound i-2a.

### Step B: Preparation of 2,2-dichloro-3-(2,5-dimethoxyphenyl)-3- phenylcyclobutanone (i-2b)

Trichloroacetyl chloride (408 μL, 3.66 mmol) was added slowly to a solution of i-2a (879 mg, 3.66 mmol) and zinc dust (239 mg, 3.66 mmol) in diethyl ether (12 mL) under sonication. After 18 h, an additional equivalent of zinc dust was added (239 mg, 3.66 mmol) followed by the slow addition of trichloroacetyl chloride (408 μL, 3.66 mmol). Upon completion of addition, the reaction mixture was poured into sat. aq. ammonium chloride and extracted three times with EtOAc. The combined organic extracts were washed with brine, dried (MgSO₄) and concentrated in vacuo. Purification of the crude residue by flash chromatography on silica gel (gradient elution; 0-25% EtOAc/hexanes as eluent) afforded the title compound i-2b.

### Step C: Preparation of 3-(2,5-dimethoxyphenyl)-3-phenylcyclobutanone (i-2c)

Zinc dust (79.0 mg, 1.21 mmol) was added to a solution of i-2b (71.0 mg, 0.20 mmol) in acetic acid (1.0 ml) at room temperature and the resulting mixture stirred at 70° C. for 8 h. After cooling to ambient temperature, the volatiles were evaporated in vacuo and the residue was partitioned between sat. aq. sodium bicarbonate and EtOAc. The organic phase was separated and the aq. phase was extracted once with EtOAc. The combined organic extracts were washed with brine, dried (MgSO₄) and concentrated in vacuo. Purification of the crude residue by flash chromatography on silica gel (gradient elution; 0-30% EtOAc/hexanes as eluent) afforded the title compound i-2c.

### Step D: Preparation of 2-(1 phenylcyclobutyl)benzene-1,4-diol (i-2d)

Intermediate i-2d can be prepared as described in Steps D and E (Scheme i-1). ¹HNMR (500 MHz, CDCl₃): δ 1.05-1.96 (m, 1H), 2.04-2.12 (m, 1H), 2.67-2.77 (m, 4H), 6.60 (d, J=1.6 Hz, 2H), 6.95 (t, J=1.6 Hz, 1H), 7.21 (tt, 1H, J=7.6, 1.2 Hz), 7.33 (m, 2H), 7.44 (m, 2H).

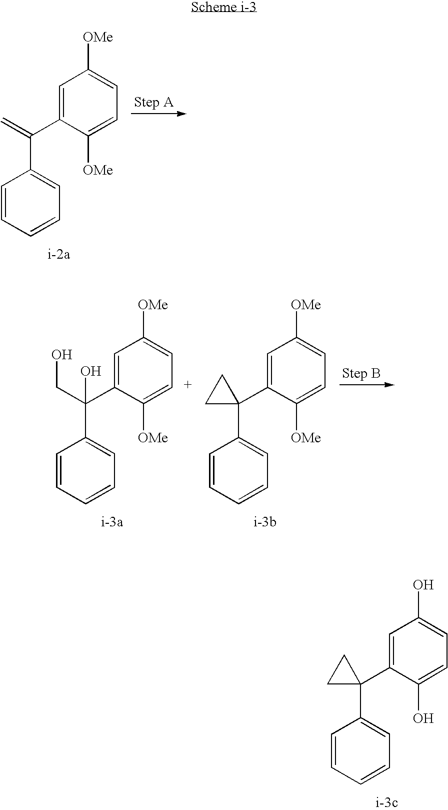

### Preparation of 2-(1-phenylcyclopropyl)benzene-1,4-diol (i-3c)

### Step A: Preparation of 1,4-dimethoxy-2-(1-phenylcyclopropyl)benzene (i-3b)

Diiodomethane (190 μL, 2.36 mmol) was added to a stirred solution of zinc/copper couple (699 mg) and copper iodide (48 mg, 0.25 mmol) in t-butylmethyl ether (30 mL) at room temperature. A solution of i-2a (500 mg, 2.08 mmol) in t-butylmethyl ether (5.5 mL) was then added via cannula and the resulting mixture heated at reflux for 18 h. After cooling to room temperature, a second portion of zinc/copper couple (622 mg), copper iodide (42 mg, 0.22 mmol) and diiodomethane (200 μL,) were added and the resulting mixture re-heated at reflux for an additional 18 h. After cooling to room temperature, the reaction mixture was filtered through a short plug of Celite®, rinsing copiously with EtOAc. The filtrate was washed twice with sat. aq. sodium bicarbonate, dried (Na₂SO₄) and concentrated in vacuo. Purification of the crude residue by flash chromatography on silica gel (isocratic elution; 60:10:1 hexanes/DCM/EtOAc as eluent) afforded an inseparable mixture of i-2a and the title compound i-3b.

N-Methylmorpholine-N-oxide (303 mg, 2.59 mmol) followed by osmium tetroxide (750 μL of a 4 wt. % solution in water, 0.118 mmol) were added to a stirred solution of the i-2a/1-3b mixture in acetone (10 mL) at room temperature. The resulting solution was aged at ambient temperature for approximately 15 h and then quenched with 10% (w/v) aq. sodium hydrogensulfite. After stirring vigorously for about 20 min, the reaction mixture was poured into water and extracted three times with EtOAc. The combined organic extracts were washed with brine, dried (Na₂SO₄) and concentrated in vacuo. Purification of the crude residue by flash chromatography on silica gel (stepwise elution; 60:10:1 hexanes/DCM/EtOAc followed by 2:1 hexanes/EtOAc as eluent) afforded in order of elution, the title compound i-3b, followed by i-3a.

### Step B: Preparation of 2-(1-phenylcyclopropyl)benzene-1,4-diol (i-3c)

Bromodimethylborane (599 uL, 6.12 mmol) was added to a stirred solution of i-3b (598 mg, 2.35 mmol) in DCM (4.0 mL) at 0° C. The resulting solution was allowed to warm to room temperature and aged for 15 h. Additional portions of bromodimethylborane (150 and 450 μL) were added after 6 h and 12 h, respectively. After cooling to 0° C., the reaction was quenched with sat. aq. sodium bicarbonate, and partitioned between EtOAc and water. The separated organic phase was washed twice with sat. aq. sodium bicarbonate, dried (Na₂SO₄) and concentrated in vacuo. Purification of the crude residue by flash chromatography on silica gel (isocratic elution; 25% EtOAc/hexanes as eluent) afforded the title compound i-3c. ¹HNMR (500 MHz, CDCl₃): δ 1.33 (dd, J=6.4, 4.4 Hz, 2H), 1.43 (dd, J=6.6, 4.3 Hz, 2H), 5.08 (s, 1H), 5.12 (s, 1H), 6.73 (dd, J=8.7, 3.0 Hz, 1H), 6.80 (d, J=8.4 Hz, 1H), 6.86 (d, J=3.0 Hz, 1H), 7.05 (dd, J=9.9, 0.5 Hz, 2H), 7.18 (brt, J=7.3 Hz, 1H), 7.27 (m, 2H).

### Preparation of 2-(1-phenylcyclohexyl)benzene-1,4-diol (i-4a)

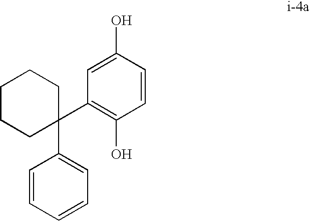

Intermediate i-4a can be prepared by using cyclopentanone in place of cyclobutanone in the synthetic procedure described above for making intermediate 1-1e (Scheme i-1). ¹HNMR (500 MHz, CDCl₃): δ 1.52 (m, 1H), 1.58 (m, 1H), 1.65 (m, 4H), 2.21-2.26 (m, 2H), 2.36-2.41 (m, 2H), 6.61 (d, J=8.5 Hz, 1H), 6.64 (dd, J=8.5, 2.7 Hz, 1H), 7.12 (d, J=2.8 Hz, 1H), 7.25 (tt, J=7.2, 1.3 Hz, 1H), 7.35 (m, 2H), 7.41 (m, 2H).

Following Procedures Similar to that Described Above for Intermediates i-1e, I-2d, 1-3c And i-4a, the Following Additional Intermediates can be Prepared:

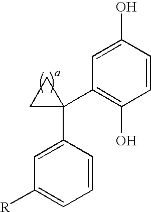

| Ex. #i-5 | a | R |
|---|---|---|
| a | 1 | F |
| b | 2 | F |
| c | 3 | F |
| d | 4 | F |

### Preparation of Nicotinoyl azide (i-6a)

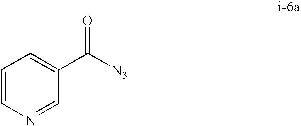

To a suspension of nicotinic acid (1.23 g, 10 mmol) in DMF (15 mL) was added diphenylphosphoryl azide (2.6 mL, 12 mmol) followed by triethylamine (1.67 mL, 12 mmol). The mixture was stirred at room temperature for 2.5 h and then poured into water (50 mL). The mixture was extracted three times with EtOAc and the combined organic extracts were washed three times with water, dried (MgSO₄) and concentrated in vacuo. Purification of the crude residue by flash chromatography on silica gel (isocratic elution; 30% EtOAc/hexanes as eluent) provided the title compound i-6a.

2-(Bromomethyl)-5-fluoroquinoline (i-7a) and 2-(Bromomethyl)-6-fluoroquinoline (i-7b) were prepared according to the procedures described in Bioorg. Med. Chem. Lett 1998, 8, 965-970.

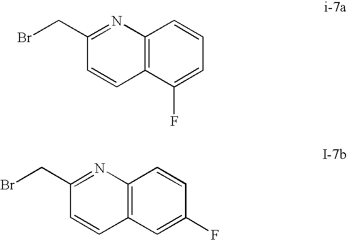

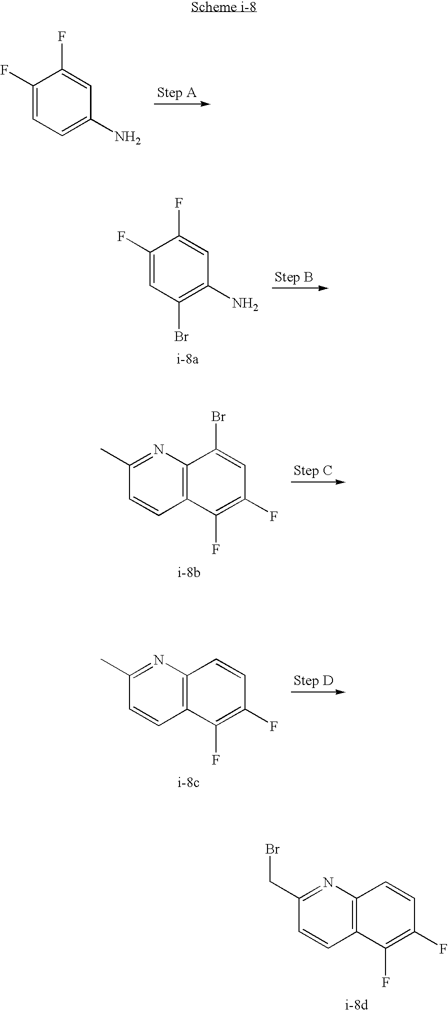

### Preparation of 2-(bromomethyl)-5,6-difluoroquinoline (i-8d)

### Step A: Preparation of (2-bromo-4,5-difluorophenyl)amine (i-8a)

Potassium carbonate (2.76 g, 20.0 mmol) was added to a stirred solution of 3,4-difluoroaniline (2.58 g, 20.0 mmol) in DCM (100 mL) at room temperature, and the resulting mixture was cooled to −15° C. A solution of bromine (3.20 g, 20.0 mmol) in DCM (10 mL) was added dropwise via syringe. After 15 min, the reaction mixture was poured into ice/water and extracted three times with DCM. The combined organic extracts were washed with water, brine, dried (MgSO₄) and concentrated in vacuo. The crude residue was purified by flash chromatography on silica gel (gradient elution; 10-20% EtOAc/Hexanes as eluent) to afford the title compound i-8a m/z (ES) 210 (MH)⁺.

### Step B: Preparation of 8-bromo-5,6-difluoro-2-methylquinoline (i-8b)

A stirred suspension of i-8a (733 mg, 4.46 mmol) in 6N hydrochloric acid (25 mL) was heated at 100° C. until the reaction mixture turned homogeneous. Toluene (6.0 mL) was added followed by dropwise addition of crotonaldehyde (740 mg, 8.92 mmol). After 3 h, the reaction mixture was cooled to room temperature and the separated aq. layer was cooled to approximately 0° C. and neutralized cautiously with 5 N aq. sodium hydroxide. The aq. phase was then extracted three times with EtOAc, the combined organic extracts were washed with water, brine, dried (MgSO₄) and concentrated in vacuo. The crude residue was purified by flash chromatography on silica gel (gradient elution; 5-10% EtOAc/Hexanes as eluent) to afford the title compound i-8b, m/z (ES) 260 (MH)⁺.

Step C: Preparation of 5,6-difluoro-2-methylquinoline (i-8c)

A mixture of i-8b (520 mg, 2.00 mmol), 2 N aq. sodium hydroxide (1.25 mL, 2.50 mmol) and palladium hydroxide on activated carbon (20%, 100 mg) was hydrogenated in EtOAc/MeOH (25 mL, 9:1) under atmospheric pressure (balloon) for 1 h. The reaction mixture was filtered through Celite® and the filtrate concentrated in vacuo. Purification of the crude residue by flash chromatography on silica gel (gradient elution; 5-25% EtOAc/Hexanes as eluent) afforded the title compound i-8c, m/z (ES) 180 (MH)⁺.

### Step D Preparation of 2-(bromomethyl)-5,6-difluoroquinoline (i-8d)

N-Bromosuccinimide (399 mg, 2.20 mmol) followed by benzoyl peroxide (50.0 mg) were added to a stirred solution of i-8c (300 mg, 1.68 mmol) in carbon tetrachloride (20 mL) at room temperature. The resulting mixture was heated to 76° C. and stirred for 3 h. After cooling to room temperature, the reaction mixture was filtered and concentrated in vacuo. Purification of the crude residue by flash chromatography on silica gel (gradient elution; 5-15% EtOAc/Hexanes as eluent) provided the title compound i-8d m/z (ES) 260 (MH)⁺. ¹H NMR (500 MHz, CDCl₃): δ 4.71 (s, 2H)), 7.60 (dd, J=18.1 Hz, 9.6 Hz, 1H), 7.67 (d, J=8.6 Hz, 1H), 7.88 (m, 1H), 8.45 (d, J=8.6 Hz, 1H).

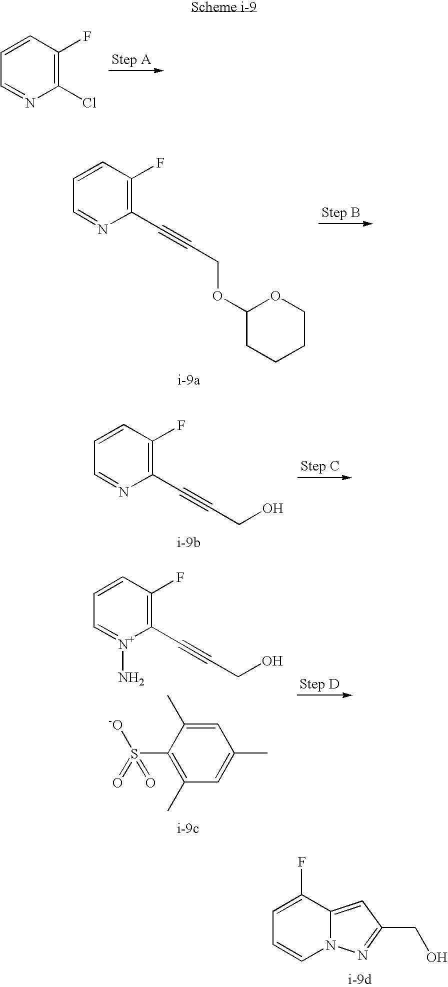

### Preparation of (4-fluoropyrazolo[1,5-a]pyridin-2-yl)methanol (i-9d)

### Step A: Preparation of 3-fluoro-2-[3-(tetrahydro-2H-pyran-2-yloxy)prop-1-yn-1-yl]pyridine (i-9a)

Tributyl[3-(tetrahydro-2H-pyran-2-yloxy)prop-1-yn-1-yl]stannane (13.8 g, 32.0 mmol, prepared according to Kyler, et al., J. Org. Chem., 1987, 52, 4296-4298) and bis(triphenylphosphine)-palladium(II) chloride (4.92 g, 6.98 mmol) were added successively to a stirred solution of 2-chloro-3-fluoropyridine (6.32 g, 48.1 mmol) in dioxane (100 mL) at room temperature. The resulting mixture was degassed with a gentle stream of nitrogen for 10 min, and then heated to 100° C. for approximately 6 h. After cooling to room temperature, the reaction mixture was quenched with sat. aq. KF and diluted with EtOAc. After stirring vigorously for approximately 15 min, the precipitated solids were removed via filtration. The organic phase was separated from the filtrate, washed with brine, dried (Na₂SO4) and concentrated in vacuo. Purification of the crude residue by flash column chromatography on silica gel (gradient elution: 10-60% EtOAc/Hexanes) gave the title compound i-9a, m/z (ES) 236 (MH)⁺.

### Step B: Preparation of 3-(3-fluoropyridin-2-yl)prop-2-yn-1-ol (i-9b)

A stirred solution of i-9a (2.20 g, 9.35 mmol) in acetic acid/water (95 mL/15 mL) was heated at 40° C. for 8 h. After cooling to room temperature, the volatiles were removed in vacuo and the residue was partitioned between EtOAc and sat. aq. sodium bicarbonate. The organic phase was separated and the aq. phase was re-extracted three times with EtOAc. The combined organic extracts were washed with water, brine, dried (MgSO₄) and concentrated in vacuo. Purification of the crude residue by the flash column chromatography on silica gel (gradient elution: 10-80% EtOAc/Hexanes) gave the title compound i-9b, m/z (ES) 134 (MH)⁺—H₂O.

### Step C: Preparation of 1-amino-3-fluoro-2-(3-hydroxyprop-1-yn-1-yl)pyridinium 2,4,6-trimethylbenzenesulfonate (i-9c)

A solution of 2-[(aminooxy)sulfonyl]-1,3,5-trimethylbenzene (1.15 g, 5.30 mmol, prepared according to Tamura, et al., Synthesis, 1977, 1-17) in DCM (15 mL) was added dropwise via syringe to a stirred solution of i-9b (536 mg, 3.55 mmol) in DCM (15 mL) at 0° C. After 2 h, the reaction mixture was warmed to room temperature, aged for 10 min and then diluted with ether (30 mL). The precipitated crystals were collected via filtration and dried in vacuo to give the title compound i-9c. ¹H NMR (500 MHz, CD₃OD): δ 2.01 (s, 3H), 2.60 (s, 6H), 4.62 (s, 2H), 6.82 (s, 2H), 7.91 (m, 1H), 8.19 (m, 1H), 8.64 (d, J=8.2 Hz, 1H).

### Step D: Preparation of (4-fluoropyrazolo[5-a]pyridin-2-yl)methanol (i-9d)

Potassium carbonate (340 mg, 2.46 mmol) was added to a stirred solution of i-9c (450 mg, 1.23 mmol) in DMF (10 mL) at room temperature. After 18 h, the reaction mixture was poured into water and extracted three times with EtOAc. The combined organic extracts were washed with water, brine, dried (MgSO₄) and concentrated in vacuo. Purification of the crude residue by flash column chromatography on silica gel (gradient elution: 20-60% EtOAc/Hexanes) gave the title compound i-9d. ¹H NMR (500 MHz, CDCl₃): 4.92 (s, 2H), 6.06 (m, 1H), 6.61 (s, 1H), 6.80 (dd, J=8.2 Hz, 8.1 Hz, 1H), 8.64 (d, J=8.2 Hz, 1H); m/z (ES) 149 (MH)⁺—H₂O.

Following procedures similar to that described above for intermediate i-9d, (5-fluoropyrazolo[1,5-a]pyridin-2-yl)methanol, (6-fluoropyrazolo[1,5-a]pyridin-2-yl)methanol, and (7-fluoropyrazolo[1,5-a]pyridin-2-yl)methanol were prepared, starting from 2-bromo-4-fluoropyridine, 2-bromo-5-fluoropyridine, and 2-bromo-6-fluoropyridine, respectively.

### Example 1

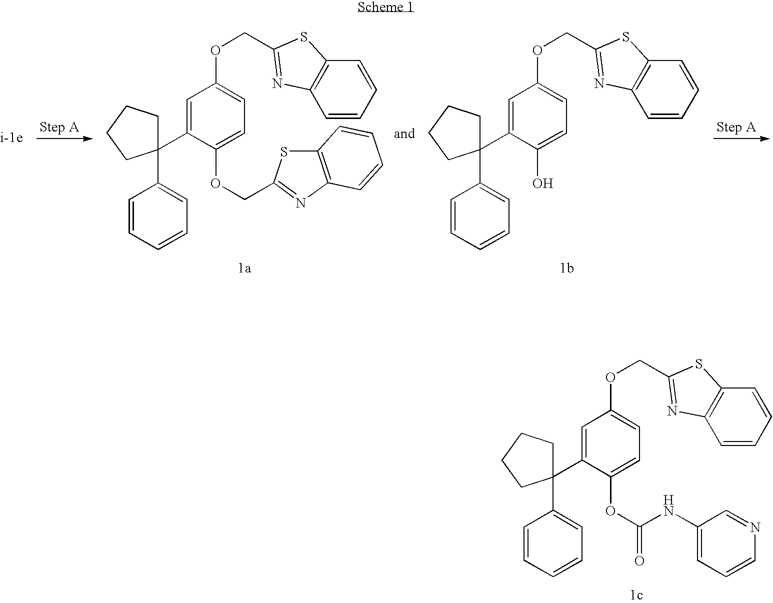

### Step A: Preparation of 2,2′-[[2-(1-phenylcyclopentyl)-1,4-phenylene]bis(oxymethylene)]bis-1,3-benzothiazole (1a) and 4-(1,3-benzothiazol-2-ylmethoxy)-2-(1-phenylcyclopentyl)phenol (1b)

2-(Chloromethyl)-1,3-benzothiazole (57.0 mg, 0.31 mmol; prepared according to Mylari, B. L.; Scott, P. J.; Zembrowski, W. J. Synth. Commun, 1989, 19, 2921-2924), potassium iodide (51.0 mg, 0.31 mmol), and potassium carbonate (71.0 mg, 0.52 mmol) were added to a stirred solution of i-1e (65.0 mg, 0.26 mmol) in DMF (0.75 mL) at room temperature. After 18 h, the reaction mixture was poured into sat. aq. ammonium chloride and extracted three times with EtOAc. The combined organic extracts were washed with water, brine, dried (MgSO₄) and concentrated in vacuo. Purification of the crude residue by flash chromatography on silica gel (gradient elution; 0-30% EtOAc/hexanes as eluent; 4% triethylamine modifier) afforded the title compounds 1a and 1b.

### Step B: Preparation of 4-(1,3-benzothiazol-2-ylmethoxy)-2-(1-iphenylcyclopentyl)phenyl pyridin-3-ylcarbamate (1c)

Nicotinoyl azide (i-6a (10.0 mg, 0.07 mmol) was heated in toluene (0.5 mL) at reflux for 30 min. A solution of 1b (17.8 mg, 0.44 mmol) in toluene was then added, followed by DIPEA, and the resulting mixture heated at reflux for 6 h. The reaction mixture was poured into water and extracted twice with EtOAc. The combined organic extracts were washed with brine, dried (MgSO₄) and concentrated in vacuo. Purification of the crude residue by flash chromatography on silica gel (gradient elution; 0-80% EtOAc/hexanes as eluent) afforded the title compound 1c, m/z (ES) 522 (MH)⁺.

Following procedures similar to that described above for Example c, the following compounds were prepared:

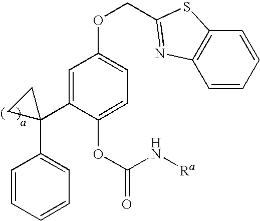

| Ex. #1 | a | R^{a} | (MH)⁺ |
|---|---|---|---|
| d | 1 | Cyclopropyl | 457 |
| e | 1 | 3-Pyridyl | 494 |
| f | 2 | Cyclopropyl | 471 |
| g | 2 | 3-Pyridyl | 508 |
| h | 3 | 3-Pyridyl | 522 |
| i | 4 | 3-Pyridyl | 536 |

In the examples shown above, the benzothiazole group can also be replaced with quinoline, 5-fluoroquinoline, 6-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 2

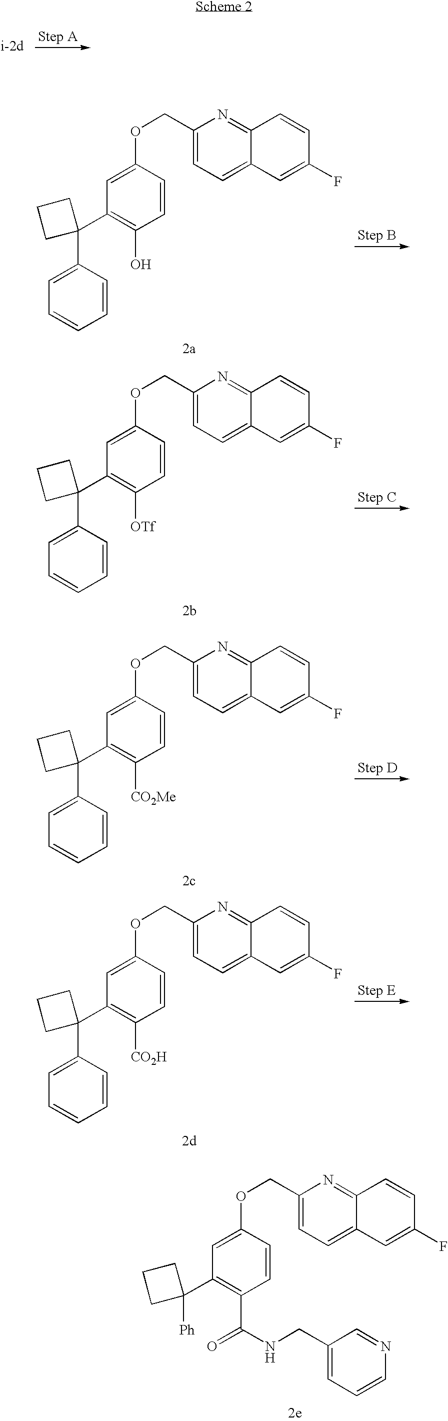

### Step A: Preparation of 4-[(6-fluoroquinolin-2-yl)methoxy]-2-(1-phenylcyclobutyl)phenol (2a)

Compound 2a can be prepared from intermediate i-2d and 2-(bromomethyl)-6-fluoroquinoline following the procedure outlined in scheme 1, step A.

### Step B: Preparation of 4-[(6-fluoroquinolin-2-yl)methoxy]-2-(1-phenylcyclobutyl)phenyl trifluoromethanesulfonate (2b)

Trifluoromethanesulfonic anhydride (1.3 equiv.) is added dropwise to a stirred solution of 2a (1.0 equiv.) in pyridine/toluene (1:1) at 0° C. The resulting mixture is allowed to warm to room temperature and then aged until the reaction is deemed complete. The reaction mixture is poured into water and extracted three times with EtOAc. The combined organic extracts are washed with water, dried (MgSO₄) and concentrated in vacuo. Purification of the crude residue by flash chromatography on silica gel affords the title compound 2b.

### Step C: Preparation of methyl 4-[(6-fluoroquinolin-2-yl methoxy]-2-(1-phenylcyclobutyl)benzoate (2c)

A stirred mixture of 2b (1 equiv.), palladium(II) acetate (0.2 equiv.), 1,1′-bis(diphenylphosphino)ferrocene (0.8 equiv.), and triethylamine (2.4 equiv.) in MeOH/DMF (1:1) is purged with carbon monoxide for approximately 10 min and then heated to 80° C. After the reaction is deemed complete, the reaction mixture is cooled to room temperature and then filtered through a short column of Celite®, eluting copiously with EtOAc. The filtrate is poured into water and the organic phase separated. The aq. phase is extracted twice with EtOAc, and the combined organic extracts are washed with brine, dried (MgSO₄) and concentrated in vacuo. Purification of the crude residue by flash chromatography on silica gel affords the title compound 2c.

### Step D: Preparation of 4-[(6-fluoroquinolin-2-yl)methoxy]-2-(1-phenylcyclobutyl)benzoic acid (2d)

Aq. potassium hydroxide (23 equiv. of a 8M solution) is added to a stirred solution of 2c (1.0 equiv.) in THF: 1,2-propanediol (1:1) and the resulting mixture heated to approximately 110° C. After the reaction is deemed complete, the reaction mixture is cooled to room temperature, acidified to about pH 6.0 with 1 N hydrochloric acid, and extracted three times with EtOAc. The combined organic extracts are washed with water, dried (MgSO₄) and concentrated in vacuo. Purification of the crude residue by preparative reversed phase HPLC on YMC Pack Pro C18 phase (gradient elution; 5-95% acetonitrile/water as eluent, 0.1% TFA as modifier). Lyophilization of the purified fractions affords the title compound 2d.

### Step E: Preparation of 4-[(6-fluoroquinolin-2-yl)methoxy]-2-(1-phenylcyclobutyl)-N-(pyridin-3-ylmethyl)benzamide (2e)

DIPEA (3.0 equiv.) is added to a stirred solution of 2d (1.0 equiv.), 3-(aminomethyl)pyridine (1.0 equiv.), and HATU (1.5 equiv.) in DMF at room temperature. After the reaction is deemed complete, the reaction mixture is poured into water and extracted three times with EtOAc. The combined organic extracts are washed with water, brine, dried (Na₂SO₄) and concentrated in vacuo. Purification of the crude residue by flash chromatography on silica gel affords the title compound 2e.

Following procedures similar to that described above for Example 2e, the following compounds can be prepared:

| | |
|---|---|
| | 2A |
| 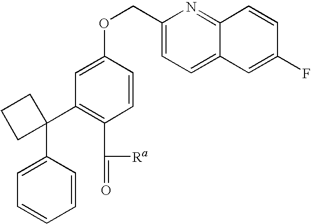 | |
| | 2B |
| 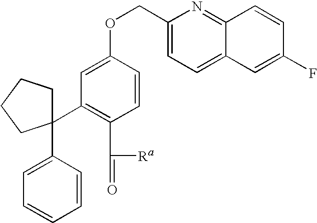 | |
| | 2C |
| 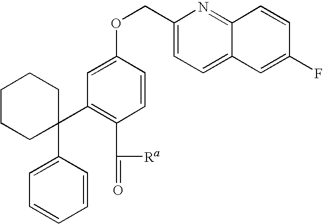 | |

| Ex. #2A | Ex. #2B | Ex. #2C | R^{a} |
|---|---|---|---|
| a | a | a | —N(H)Me |
| b | b | b | —NMe₂ |
| c | c | c | —N(H)Et |
| d | d | d | —NEt₂ |
| e | e | e | —N(H)Pr |
| f | f | f | —N(H)iPr |
| g | g | g | —N(H)cyclopropyl |
| h | h | h | —N(Me)Et |
| i | i | i | 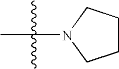 |
| j | j | j | 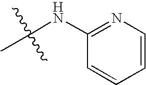 |
| k | k | k | 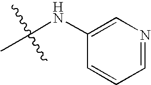 |
| l | l | l | 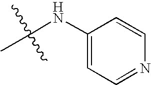 |
| m | m | m | 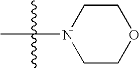 |
| n | n | n | 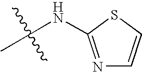 |
| o | o | o | 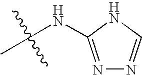 |
| — | p | p | —OMe |
| — | q | q | —OH |

In the examples shown above, the 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 3

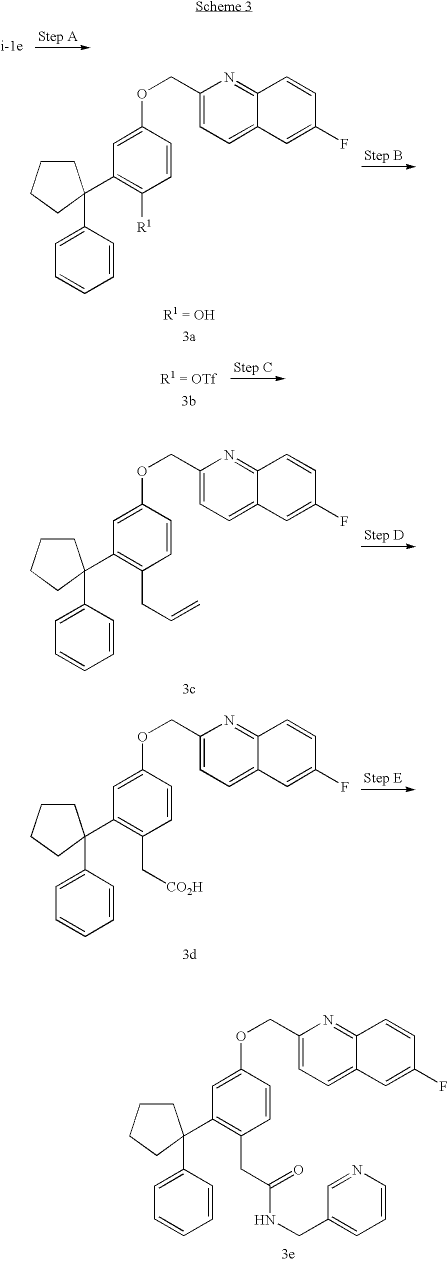

### Step A: Preparation of 4-[(6-fluoroquinolin-2-yl)methoxy]-2-(1-phenylcyclopentyl)phenol (3a)

Compound 3a can be prepared from intermediate i-1e and 2-(bromomethyl)-6-fluoroquinoline following the procedure outlined in scheme 1, step A.

### Step B: Preparation of 4-[(6-fluoroquinolin-2-yl)methoxy]-2-(1 phenylcyclopentyl)phenyl Trifluoromethanesulfonate (3b)

Compound 3b can be prepared from intermediate 3a following the procedure outlined in scheme 2, step B.

### Step C: Preparation of 2-{[4-allyl-3-(1-phenylcyclopentyl)phenoxy]methlyl}-6-fluoroquinoline (3c)

Lithium chloride (5.0 equiv.), [1,1′-bis(diphenylphosphino)ferrocene]-dichloropalladium(II) (0.016 equiv.), and allyl(tributyl)stannane (2.0 equiv.) are added to a solution of 3a in 1-methyl-2-pyrrolidinone and the resulting mixture is irradiated in a microwave apparatus (300 W) at 120° C. until the reaction is deemed complete. The reaction mixture is diluted with EtOAc and treated with 1,8-diazabicyclo[5.4.0]unced-7-ene (6.6 equiv.) for approximately 20 min. The reaction mixture is filtered through silica, and the filtrate stirred with sat. aq. potassium fluoride at 50° C. for 24 h. The organic phase is separated and the aq. phase extracted three times with EtOAc. The combined organic extracts are washed with brine, dried (MgSO₄) and concentrated in vacuo. Purification of the crude residue by flash chromatography on silica gel affords the title compound 3c.

### Step D: Preparation of [4-[(6-fluoroquinolin-2-yl)methoxy]-2-(1-phenylcyclopentyl)phenyl]acetic Acid (3d)

Sodium periodate (4.1 equiv.) and ruthenium(III) chloride (0.01 equiv.) are added to a solution of 3c in carbon tetrachloride-water-acetonitrile and the resulting solution allowed to stir at room temperature for 1 h adding more sodium periodate and ruthenium(III) chloride if necessary. After the reaction is deemed complete, the reaction mixture is poured into water and extracted three times with DCM. The combined organic extracts are washed with brine, dried (MgSO₄) and concentrated in vacuo. Purification of the crude residue by flash chromatography on silica gel affords the title compound 3d.

### Step E: Preparation of 2-[4-[(6-fluoroquinolin-2-yl)methoxy]-2-(1-phenylcyclopentyl)phenyl]-N-(pyridin-3-ylmethyl)acetamide (3e)

DIPEA (3.0 equiv.) is added to a stirred solution of 3d (1.0 equiv.), 3-(aminomethyl)pyridine (1.0 equiv.), and HATU (1.5 equiv.) in DMF at room temperature. After the reaction is deemed complete, the reaction mixture is poured into water and extracted three times with EtOAc. The combined organic extracts are washed with water, brine, dried (MgSO₄) and concentrated in vacuo. Purification of the crude residue by flash chromatography on silica gel affords the title compound 3e.

Following procedures similar to that described above for Example 3d, the following compounds can be prepared:

| | |
|---|---|
| | 3A |
| 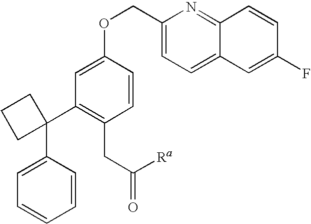 | |
| | 3B |
| 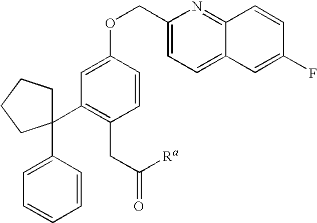 | |
| | 3C |
| 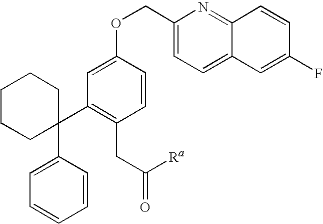 | |

| Ex. #3A | Ex. #3B | Ex. #3C | R^{a} |
|---|---|---|---|
| a | a | a | —N(H)Me |
| b | b | b | —NMe₂ |
| c | c | c | —N(H)Et |
| d | d | d | —NEt₂ |
| e | e | e | —N(H)Pr |
| f | f | f | —N(H)iPr |
| g | g | g | —N(H)cyclopropyl |
| h | h | h | —N(Me)Et |
| i | i | i | 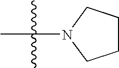 |
| j | j | j | 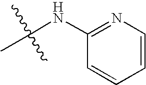 |
| k | k | k | 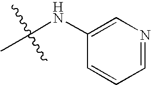 |
| l | l | l | 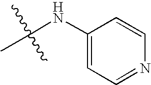 |
| m | m | m | 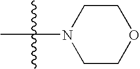 |
| n | n | n | 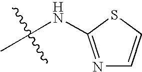 |
| o | o | o | 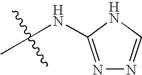 |
| — | p | p | —OMe |
| — | q | q | —OH |

In the examples shown above, the 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 4

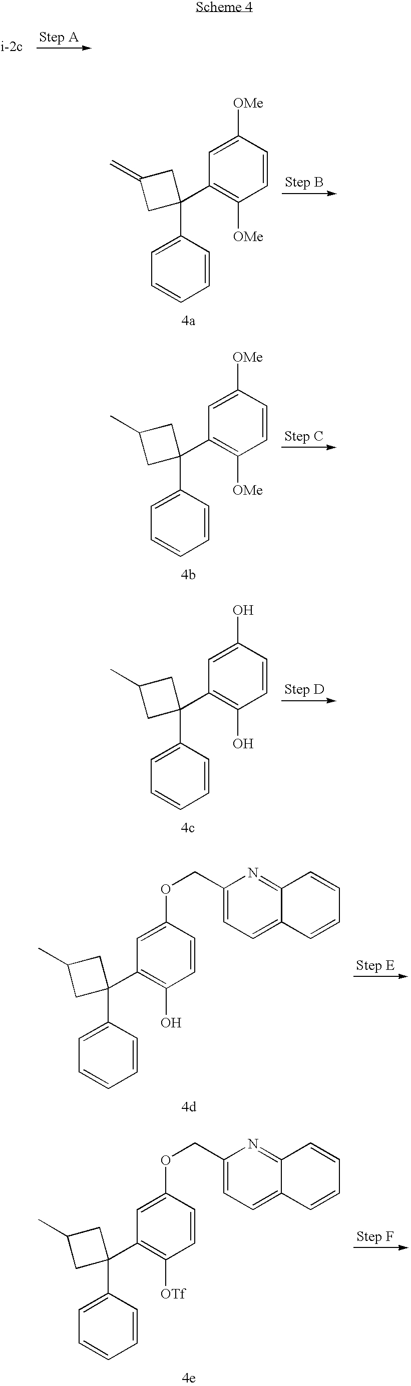

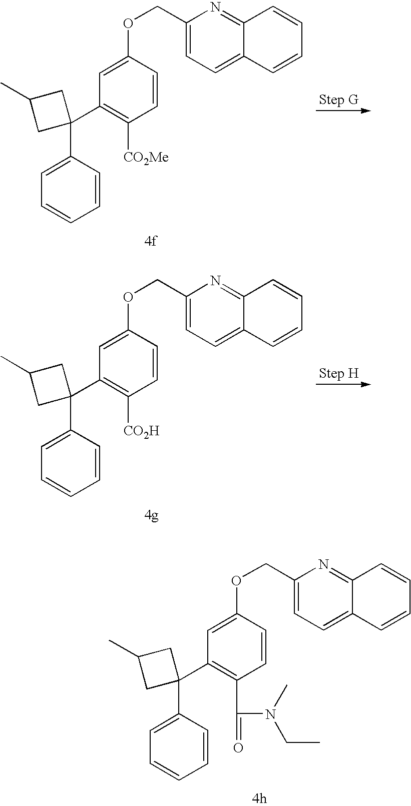

### Step A: Preparation of 1,4-dimethoxy-2-(3-methylene-1-phenylcyclobutyl)-benzene (4a)

Potassium bis(trimethylsilyl)amide (7.27 mL of a 0.5 M solution in toluene, 3.64 mmol) was added to a solution of methyltriphenylphosphonium bromide (1.30 g, 3.63 mmol) in THF (20 mL) at approximately 0° C. After 30 min, a solution of i-2c (0.73 g, 2.59 mmol) in THF (5 mL) was added dropwise via syringe, and the resulting mixture allowed to warm to room temperature. After approximately 4 h, the reaction mixture was poured into water and extracted three times with EtOAc. The combined organic extracts were washed with brine, dried (MgSO₄) and concentrated in vacuo. Purification of the crude residue by flash chromatography on silica gel (gradient elution; 0-20% EtOAc/hexanes as eluent) afforded the title compound 4a, m/z (ES) 281 (MH)⁺. ¹HNMR (500 MHz, CDCl₃): δ 3.42 (m, 2H), 3.52 (m, 2H), 3.68 (s, 3H), 3.87 (s, 3H), 4.91 (quin., J=2.3 Hz, 2H), 6.81, (m 2H), 7.00 (d, J=2.5 Hz, 1H), 7.19 (t, J=7.3 Hz, 1H), 7.31 (t, J=7.3 Hz, 2H), 7.43 (d, J=7.6 Hz, 2H).

### Step B: Preparation of 1,4-dimethoxy-2-(3-methyl-1-phenylcyclobutyl)benzene (4b)

A mixture of (4a) (670 mg, 2.39 mmol) palladium (66.0 mg of 10 wt. % on activated carbon) in EtOH (15 mL) was hydrogenated at atmospheric pressure for approximately 15 h. The resulting mixture was filtered through a short column of Celite®, eluting copiously with EtOAc. The filtrate was concentrated in vacuo to give 4b as a 1:1 mixture of cis/trans diastereomers which was used without further purification in the subsequent reaction, m/z (ES) 282 (MH)⁺.

### Step C: Preparation of 2-(3-methyl-1-phenylcyclobutyl)benzene-1,4-diol (4c)

Boron tribromide (7.20 mL of a 1 M solution in DCM, 7.20 mmol) was added dropwise to a stirred solution of crude 4b (2.39 mmol) in DCM at approximately 0° C. The resulting mixture was allowed to warm to room temperature and aged for 2.5 d. The reaction mixture was poured into water and extracted three times with EtOAc. The combined organic extracts were washed with brine, dried (MgSO₄) and concentrated in vacuo. Purification of the crude residue by flash chromatography on silica gel (gradient elution; 0-30% EtOAc/hexanes as eluent) furnished the title compound 4c, as a 1:1 mixture of cis/trans diastereomers. ¹HNMR (500 MHz, CDCl₃): δ 1.13 (d, J=3.4 Hz, 3H), 1.14 (d, J=3.4 Hz, 3H), 2.32 (m, 4H), 2.40 (m, 1H), 2.53 (m, 1H), 2.90 (m, 4H), 4.11 (s, 1H), 4.16 (s, 1H), 4.51 (s, 1H), 4.54 (s, 1H), 6.59 (s, 1H), 6.60 (s, 1H), 6.67 (s, 1H), 6.68 (s, 1H), 6.87 (m, 1H), 7.15 (t, J=1.1 Hz, 1H), 7.23 (m, 2H), 7.33 (m, 6H), 7.50 (m, 2H).

### Step D: Preparation of 2-(3-methyl-1-phenylcyclobutyl)-4-(quinolin-2-ylmethoxy)phenol (4d)

2-(Chloromethyl)quinoline (505 mg, 2.84 mmol), followed by potassium iodide (473 mg, 2.85 mmol) and potassium carbonate (603 mg, 4.36 mmol) were added to a stirred solution of 4c (557 mg, 2.19 mmol) in DMF (3 mL) at room temperature. After approximately 15 h, the reaction mixture was diluted with water and acidified to pH 6 with 1 N hydrochloric acid. The aq. phase was extracted three times with EtOAc, washed with brine, dried (MgSO₄) and concentrated in vacuo. Purification of the crude residue by flash chromatography on silica gel (gradient elution; 0-35% EtOAc/hexanes, 4% triethylamine modifier) provided the title compound 4d, m/z (ES) 396 (MH)⁺.

### Step E: Preparation of 2-(3-methyl-1-phenylcyclobutyl)-4-(quinolin-2-ylmethoxy)phenyl Trifluoromethanesulfonate (4e)

Sodium hydride (23.0 mg, 0.96 mmol) was added to a stirred solution of 4d (322 mg, 0.81 mmol) in THF (8 mL) at approximately 0° C. After 20 min, 2-[N,N-bis(trifluoromethylsulfonyl)amino]-5-chloropyridine (480 mg, 1.22 mmol) was added, and the resulting mixture allowed to warm to room temperature. After approximately 30 min, the reaction mixture was poured into water and extracted three times with EtOAc. The combined organic extracts were washed with brine, dried (MgSO₄) and concentrated in vacuo. Purification of the crude residue by flash chromatography (gradient elution; 0-20% EtOAc/hexanes as eluent) afforded the title compound 4e, m/z (ES) 528 (MH)⁺.

### Step F: Preparation of methyl 2-(3-methyl-1-phenylcyclobutyl)-4-(quinolin-2-ylmethoxy)benzoate (4f)

A stirred mixture of 4e (352 mg, 0.67 mmol), palladium(II) acetate (30.0 mg, 0.13 mmol), 1,1′-bis(diphenylphosphino)ferrocene (296 mg, 0.53 mmol), and triethylamine (223 μL, 1.60 mmol) in MeOH (3 mL) and DMF (3 mL) was purged with carbon monoxide for approximately 10 min and then heated to 80° C. After 1 d, the reaction mixture was cooled to room temperature and then filtered through a short column of Celite®, eluting copiously with EtOAc. The filtrate was poured into water and the organic phase separated. The aq. phase was re-extracted twice with EtOAc, and the combined organic extracts were washed with brine, dried (MgSO₄) and concentrated in vacuo. Purification of the crude residue by flash chromatography on silica gel (gradient elution; 0-15% EtOAc/hexanes as eluent) furnished the title compound 4f, m/z (ES) 438 (MH)⁺. Mixture 4f was resolved into its diastereoisomeric components by preparative chiral HPLC (Chiralpak AD column, 5% isopropanol/heptane as eluent) to provide in order of elution:

4f-A (Diastereoisomer A): retention time=13.66 min on analytical Chiralpak AD column (4.6×250 mm; 10 micron, flow rate=0.75 mL/min, λ=254 nm TV detection); ¹HNMR (500 MHz, CDCl₃): δ 1.04 (d, J=6.4 Hz, 3H), 2.20 (m, 2H), 2.45 (m, 1H), 2.93 (m, 2H), 3.71 (s, 3H), 5.52 (s, 2H), 6.91 (dd, J=8.7, 2.5 Hz, 1H), 7.14 (t, J=7.3 Hz, 1H), 7.17 (d, J=2.6 Hz, 1H), 7.22 (t, J=7.4 Hz, 2H), 7.42 (d, J=8.7 Hz, 2H), 7.64 (m, 2H), 7.72 (d, J=8.5 Hz, 1H), 7.80 (t, J=7.8 Hz, 1H), 7.90 (d, J=8.0 Hz, 1H), 8.14 (d, J=8.7 Hz, 1H), 8.26 (d, J=8.5 Hz, 1H).

4f-B (Diastereoisomer B): retention time=16.39 min on analytical Chiralpak AD column (4.6×250 mm; 10 micron, flow rate=0.75 mL/min, λ=254 nm UV detection); ¹HNMR (500 MHz, CDCl₃): δ 1.09 (d, J=6.6 Hz, 3H), 2.24 (octet, J=6.9 Hz, 1H), 2.42 (m, 2H), 2.87 (m, 2H), 3.55 (s, 3H), 5.54 (s, 2H), 6.96 (dd, J=8.7, 2.6 Hz, 1H), 7.12 (m, 1H), 7.21 (m, 4H), 7.50 (d, J=2.5 Hz, 1H), 7.62 (t, J=7.8 Hz, 1H), 7.70 (d, J=8.7 Hz, 1H), 7.75 (d, J=8.5 Hz, 1H), 7.80 (t, J=7.1 Hz, 1H), 7.90 (d, J=8.0 Hz, 1H), 8.15 (d, J=8.5 Hz, 1H), 8.27 (d, J=8.5 Hz, 1H).

### Step G: Preparation of 2-(3-methyl-1-phenylcyclobutyl)-4-(quinolin-2-ylmethoxy)benzoic Acid (4g)

Aq. potassium hydroxide (187 μL of a 8 M solution, 1.50 mmol) was added to a stirred solution of 4f-B (28.5 mg, 0.065 mmol) in THF (1.5 mL) and propylene glycol (1.5 mL) and the resulting mixture heated to approximately 110° C. After approximately 15 h, the reaction mixture was cooled to room temperature, diluted with water, and then acidified to pH 6 with 1 N hydrochloric acid. The aq. phase was extracted three times with EtOAc, and the combined organic extracts were washed with brine, dried (MgSO₄) and concentrated in vacuo. Purification of the crude residue by preparative reversed phase HPLC on YMC Pack Pro C18 phase (gradient elution; 5-95% acetonitrile/water as eluent, 0.1% TFA modifier). Lyophilization of the purified fractions provided the title compound 4g, m/z (ES) 424 (MH)⁺. ¹HNMR (500 MHz, CDCl₃): δ 1.09 (d, J=6.6 Hz, 3H), 2.23 (m, 1H), 2.46 (m, 2H), 2.90 (m, 2H), 5.70 (s, 2H), 6.70 (dd, J=8.9, 2.5 Hz, 1H), 7.11 (m, 1H), 7.22 (m, 3H), 7.53 (d, J=2.5 Hz, 1H), 7.73 (t, J=7.6 Hz, 1H), 7.90 (m, 4H), 7.99 (d, J=7.6 Hz, 1H), 8.32 (d, J=8.6 Hz, 1H), 8.46 (d, J=8.3 Hz, 1H).

### Step H: Preparation of N-ethyl-N-methyl-2-(3-methyl-1-phenylcyclobutyl)-4-(quinolin-2-ylmethoxy)benzamide (4h)

A mixture of 4g (9.6 mg, 23 μmol), HATU (17 mg, 45 μmol), N-ethylmethylamine (19 μL, 23 μmol) and DIPEA (39 μL, 230 μmol) in DMF (1.1 mL) was stirred at room temperature for approximately 15 h. The reaction mixture was poured into sat. aq. bicarbonate and extracted three times with EtOAc. The combined organic extracts were washed with brine, dried (MgSO₄) and concentrated in vacuo. Purification of the crude residue by flash chromatography on silica gel (gradient elution; 0-50% EtOAc/hexanes as eluent) furnished the title compound 4h, as a mixture of rotamers, m/z (ES) 465 (MH)⁺. ¹HNMR (500 MHz, CDCl₃): δ0.78 (t, J=7.1 Hz, 1.5H), 0.97 (d, J=6.6 Hz, 1.5H), 0.99 (d, J=6.6 Hz, 1.5H), 1.07 (t, J=7.1 Hz, 1.5H), 1.55 (sext., J=7.3 Hz, 0.5H), 1.87 (m, 0.5H), 1.92 (s, 1.5H), 2.26 (m, 1H), 2.34 (m, 1H), 2.58 (m, 1H), 2.70 (s, 1.5H), 2.88 (m, 0.5H), 2.97 (m, 1H), 3.18 (m, 1H), 3.63 (m, 0.5H), 5.53 (s, 2H), 6.88 (dd, J=8.2, 2.5 Hz, 1H), 6.94 (d, J=8.2 Hz, 1H), 7.00 (d, J=8.3 Hz, 1H), 7.09 (m, 1H), 7.19 (m, 3H), 7.51 (dd, J=11.5, 2.3 Hz, 1H), 7.62 (t, J=7.6 Hz, 1H), 7.77 (dd, J=8.5, 3.0 Hz, 1H), 7.80 (t, J=7.3 Hz, 1H), 7.90 (d, J=8.3 Hz, 1H), 8.17 (d, J=8.2 Hz, 1H), 8.28 (d, J=8.5 Hz, 1H).

Following procedures similar to that described above for Example 4h, the following compounds can be prepared (*compounds can be either racemic or chiral):

| | |
|---|---|
| | 4A |
| 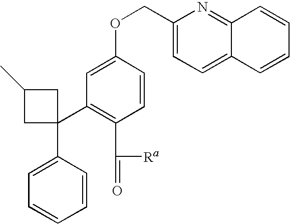 | |
| | 4B |
| 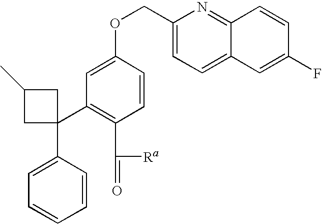 | |

| Ex. #4A* | Ex. #4B* | R^{a} |
|---|---|---|
| a | a | —N(H)Me |
| b | b | —NMe₂ |
| c | c | —N(H)Et |
| d | d | —NEt₂ |
| e | e | —N(H)Pr |
| f | f | —N(H)iPr |
| g | g | —N(H)cyclopropyl |
| h | h | —N(Me)Et |
| i | i | 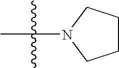 |
| j | j | 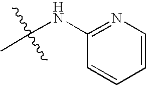 |
| k | k | 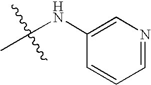 |
| l | l | 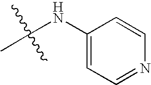 |
| m | m | 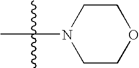 |
| n | n | 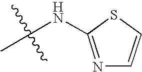 |
| o | o | 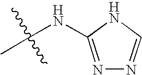 |
| — | p | -OMe |
| — | q | —OH |

In the examples shown above, the quinoline or 6-fluoroquinoline groups can also be replaced with 5-fluoroquinoline, 6-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 5

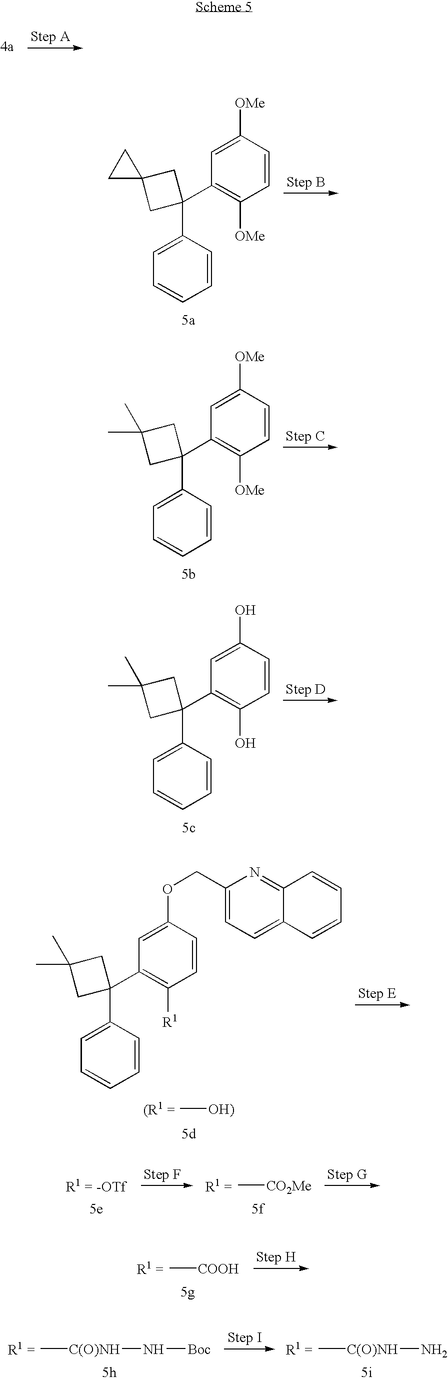

### Step A: Preparation of 5-(2,5-dimethoxyphenyl)-5-phenylspiro[2,3]hexane (5a)

Diethyl zinc (3.02 mL of a 1 M solution in hexanes, 3.02 mmol) followed by chloroiodomethane (438 μL, 6.04 mmol) were added to a stirred solution of 4a (425 mg, 1.51 mmol) in dichloroethane at 0° C. After approximately 2 h, the reaction was quenched by the addition of sat. aq. ammonium chloride. The resulting mixture was poured into water and extracted three times with DCM. The combined organic extracts were washed with brine, dried (MgSO₄) and concentrated in vacuo. Purification of the crude residue by flash chromatography (gradient elution; 0-15% EtOAc/hexanes as eluent) afforded the title compound 5a. ¹HNMR (500 MHz, CDCl₃): δ 0.44 (d, J=9.8 Hz, 1H), 0.45 (d, J=9.0 Hz, 1H), 0.57 (d, J=9.0 Hz, 1H), 0.59 (d, J=9.8 Hz, 1H), 2.74 (dd, J=13.2, 8.8 Hz, 2H), 3.06 (dd, J=13.2, 8.8 Hz, 2H), 3.63 (s, 3H), 3.84 (s, 3H), 6.77 (m, 2H), 7.00 (d, J=2.5 Hz, 1H), 7.17 (t, J=7.3 Hz, 1H), 7.30 (t, J=8.3 Hz, 2H), 7.48 (d, J=8.3 Hz, 2H).

### Step B: Preparation of 2-(3,3-dimethyl-1-phenylcyclobutyl)-1,4-dimethoxybenzene (5b)

A mixture of (5a) (376 mg, 1.28 mmol) platinum (245 mg of 5 wt. % on activated carbon) in MeOH (7 mL) and acetic acid (2 mL) was hydrogenated at atmospheric pressure for approximately 24 h. The resulting mixture was filtered through a short column of Celite®, eluting copiously with DCM. The filtrate was concentrated in vacuo to give 5b, which was used without further purification in the subsequent reaction. ¹HNMR (500 MHz, CDCl₃): δ 1.00 (s, 3H), 1.08 (s, 3H), 2.68 (d, J=12.8 Hz, 2H), 2.78 (d, J=12.8 Hz, 2H), 3.69 (s, 3H), 3.81 (s, 3H), 6.68 (m, 2H), 7.0 (d, J=2.7 Hz, 1H), 7.10 (t, J=9.3 Hz, 1H), 7.25 (t, J=7.8 Hz, 2H), 7.48 (d, J=7.8 Hz, 1H).

### Step C: Preparation of 2-(3,3-dimethyl-1-phenylcyclobutyl)benzene-1,4-diol (5c)

Boron tribromide (3.71 mL of a 1 M solution in DCM, 3.71 mmol) was added dropwise to a stirred solution of 5b in DCM at approximately 0° C. After 24 h, the reaction mixture was poured into water and extracted three times with DCM. The combined organic extracts were washed with brine, dried (MgSO₄) and concentrated in vacuo. Purification of the crude residue by flash chromatography on silica gel (gradient elution; 0-30% EtOAc/hexanes as eluent) furnished the title compound 5c. ¹HNMR (500 MHz, CDCl₃): δ 1.04 (s, 3H), 1.05 (s, 3H), 2.68 (d, J=11.3 Hz, 2H), 2.72 (d, J=11.3 Hz, 2H), 6.60 (m, 2H), 6.95 (s, 1H), 7.18 (t, J=7.4 Hz, 1H), 7.32 (d, J=7.4 Hz, 2H), 7.42 (d, J=7.4 Hz, 2H). [2×OH not found]

### Step D: Preparation of 2-(3,3-dimethyl-1-phenylcyclobutyl)-4-(quinolin-2-ylmethoxy)phenol (5d)

2-(Chloromethyl)quinoline (226 mg, 1.27 mmol), followed by potassium iodide (211 mg, 1.27 mmol) and potassium carbonate (270 mg, 1.96 mmol) were added to a stirred solution of 5c (263 mg, 0.98 mmol) in DMF (1.4 mL) at room temperature. After approximately 5 h, the reaction mixture was diluted with water and acidified to pH 6 with 1 N hydrochloric acid. The aq. phase was extracted three times with EtOAc, washed with brine, dried (MgSO₄) and concentrated in vacuo. Purification of the crude residue by flash chromatography on silica gel (gradient elution; 0-40% EtOAc/hexanes, 4% triethylamine in each phase as modifier) provided the title compound 5d. ¹HNMR (500 MHz, CDCl₃): δ 1.05 (s, 3H), 1.07 (s, 3H), 2.68 (d, J=11.3 Hz, 2H), 2.72 (d, J=11.3 Hz, 2H), 4.50 (s, 1H), 5.40 (s, 2H), 6.62 (d, J=8.1 Hz, 1H), 6.72 (dd, J=8.1 Hz, 1H), 7.14 (t, J=7.4 Hz, 1H), 7.17 (d, J=3.7 Hz, 1H), 7.24 (t, J=7.4 Hz, 2H), 7.38 (d, J=7.4 Hz, 2H), 7.62 (t, J=7.4 Hz, 1H), 7.78 (m, 2H), 7.89 (d, J=8.1 Hz, 1H), 8.15 (s, 1H), 8.26 (d, J=8.1 Hz, 1H).

### Step E: Preparation of 2-(3,3-dimethyl-1 phenylcyclobutyl)-4-(quinolin-2-ylmethoxy)phenyl Trifluoromethanesulfonate (5e)

Sodium hydride (29.0 mg of a 60% dispersion in mineral oil, 0.72 mmol) was added to a stirred solution of 5d (184 mg, 0.45 mmol) in THF (6.2 mL) at approximately 0° C. After 30 min, 2-[N,N-bis(trifluoromethylsulfonyl)amino]-5-chloropyridine (318 mg, 0.81 mmol) was added, and the resulting mixture maintained at 0° C. for approximately 4 h. The reaction mixture was poured into water and extracted three times with EtOAc. The combined organic extracts were washed with brine, dried (MgSO₄) and concentrated in vacuo. Purification of the crude residue by flash chromatography (gradient elution; 0-30% EtOAc/hexanes as eluent) afforded the title compound 5e. ¹HNMR (500 MHz, CDCl₃): δ 0.99 (s, 3H), 1.05 (s, 3H), 2.66 (d, J=12.8 Hz, 2H), 2.85 (d, J=10.7 Hz, 2H), 5.45 (s, 2H), 6.84 (dd, J=9.1, 3.2 Hz, 1H), 7.09 (s, 1H), 7.11 (t, J=7.1 Hz, 1H), 7.19 (t, J=7.8 Hz, 2H), 7.26, (d, J=3.2 Hz, 1H), 7.34 (d, J=7.3 Hz, 2H), 7.62, (t, J=7.1 Hz, 1H), 7.69 (d, J=8.5 Hz, 1H), 7.80 (t, J=8.2 Hz, 1H), 7.89 (d, J=8.0 Hz, 1H), 8.16 (d, J=8.3 Hz, 1H), 8.25 (d, J=8.3 Hz, 1H).

### Step F: Preparation of methyl 2-(3,3-dimethyl-1-phenylcyclobutyl)-4-(quinolin-2-ylmethoxy)benzoate (5f)

A stirred mixture of 5e (146 mg, 0.27 mmol), palladium(II) acetate (61.0 mg, 0.27 mmol), 1,1′-bis(diphenylphosphino)ferrocene (299 mg, 0.54 mmol), and triethylamine (90.0 μL, 0.65 mmol) in MeOH (2 mL) and DMF (2 mL) was purged with carbon monoxide for approximately 10 min and then heated to 80° C. After approximately 16 h, the reaction mixture was cooled to room temperature and then filtered through a short column of Celite®, eluting copiously with EtOAc. The filtrate was poured into water and the organic phase separated. The aq. phase was re-extracted twice with EtOAc, and the combined organic extracts were washed with brine, dried (MgSO₄) and concentrated in vacuo. Purification of the crude residue by flash chromatography on silica gel (gradient elution; 0-30% EtOAc/hexanes as eluent) furnished the title compound 5f, m/z (ES) 452 (MH)⁺. ¹HNMR (500 MHz, CDCl₃): δ 0.95 (s, 3H), 1.04 (s, 3H), 2.54 (d, J=13.1 Hz, 2H), 2.77 (d, J=13.0 Hz, 2H), 3.71 (s, 3H), 5.51 (s, 2H), 6.89 (dd, J=8.7, 2.5 Hz, 1H), 7.08 (t, J=7.3 Hz, 1H), 7.16 (t, J=7.6 Hz, 1H), 7.29 (s, 1H), 7.40 (d, J=7.3 Hz, 2H), 7.61 (t, J=7.8 Hz, 1H), 7.64 (d, J=8.4 Hz, 1H), 7.69 (d, J=8.5 Hz, 1H), 7.79 (t, J=7.1 Hz, 1H), 7.88 (d, J=7.8 Hz, 1H), 8.14 (d, J=8.3 Hz, 1H), 8.23 (d, J=8.5 Hz, 1H).

### Step G: Preparation of 2-(3,3-dimethyl-1-phenylcyclobutyl)-4-(quinolin-2-ylmethoxy)benzoic Acid (5g)

Aq. potassium hydroxide (526 μl of a 8 M solution, 4.21 mmol) was added to a stirred solution of 5f (83.0 mg, 0.18 mmol) in THF (1.8 mL) and propylene glycol (1.8 mL) and the resulting mixture heated to approximately 110° C. After approximately 15 h, the reaction mixture was cooled to room temperature, diluted with water and then acidified to pH 6 with 1 N hydrochloric acid. The aq. phase was extracted three times with EtOAc, and the combined organic extracts were washed with brine, dried (MgSO₄) and concentrated in vacuo. Crude 5g (m/z (ES) 438 (MH)⁺) was used without further purification in the subsequent reaction.

### Step H: Preparation of tert-butyl 2-[2-(3,3-dimethyl-1-phenylcyclobutyl)-4-(quinolin-2-ylmethoxy)benzoyl]hydrazinecarboxylate (5h)

A mixture of crude 51z (0.18 mmol), HATU (139 mg, 0.37 mmol), tert-butyl carbazate (121 mg, 0.92 mmol) and DIPEA (159 μL, 0.92 mmol) in DMF was stirred at room temperature for approximately 1 h. The reaction mixture was poured into sat. aq. bicarbonate and extracted three times with DCM. The combined organic extracts were washed with brine, dried (MgSO₄) and concentrated in vacuo. Purification of the crude residue by flash chromatography on silica gel (gradient elution; 0-50% EtOAc/hexanes as eluent) furnished the title compound 5h, m/z (ES) 552 (MH)⁺. ¹HNMR (500 MHz, CDCl₃): δ 0.98 (s, 3H), 1.00 (s, 3H), 1.49 (s, 9H), 2.65 (d, J=12.8 Hz, 2H), 2.72 (d, J=12.8 Hz, 2H), 2.82 (s, 1H), 5.50 (s, 2H), 6.42 (br s, 1H), 6.87 (dd, J=8.5, 2.6 Hz, 2H), 7.09 (t, J=7.3 Hz, 1H), 7.17 (t, J=7.3 Hz, 2H), 7.25 (d, J=2.5 Hz, 1H), 7.39 (d, J=7.3 Hz, 2H), 7.61 (t, J=7.6 Hz, 1H), 7.69 (d, J=8.2 Hz, 1H), 7.80 (t, J=7.3 Hz, 1H), 7.88 (d, J=8.0 Hz, 1H), 8.16 (br s, 1H), 8.25 (d, J=8.2 Hz, 1H).

### Step I: Preparation of 2-(3,3-dimethyl-1-phenylcyclobutyl)-4-(quinolin-2-ylmethoxy)benzo-hydrazide (5i)

Hydrogen chloride (3 mL of a 1 M solution in diethyl ether, excess) was added to a stirred solution of 5h (99.3 mg, 0.18 mmol) in DCM (3 mL) at room temperature. The reaction mixture became heterogeneous almost instantly. After 1 h, a second portion of hydrogen chloride (4 mL of a 4M solution in dioxane) was added. After approximately 3 h, the reaction mixture was concentrated in vacuo and the crude residue partitioned between sat. aq. sodium bicarbonate and DCM. The organic phase was separated, and the aq. phase was extracted twice with DCM. The combined organic extracts were washed with brine, dried (MgSO₄) and concentrated in vacuo to afford crude 51 (Q/z (ES) 452 (MH)⁺. ¹HNMR (500 MHz, CDCl₃): δ 0.96 (s, 3H), 1.04 (s, 3H), 2.56 (d, J=13 Hz, 2H), 2.74 (d, J=13.1 Hz, 2H), 5.55 (s, 2H), 6.95 (dd, J=8.5, 2.6 Hz, 1H), 7.07 (t, J=7.1 Hz, 1H), 7.15 (t, J=7.3 Hz, 2H), 7.31 (d, J=7.6 Hz, 2H), 7.38 (d, J=2.5 Hz, 1H), 7.47 (d, J=8.7 Hz, 1H), 7.63 (t, J=7.6 Hz, 1H), 7.73 (d, J=8.5 Hz, 1H), 7.81 (t, J=7.3 Hz, 1H), 7.90 (d, J=8.0 Hz, 1H), 8.19 (br s, 1H), 8.28 (d, J=8.2 Hz, 1H), 9.74 (s, 1H).

Following procedures similar to that described above for Example 5h, the following compounds can be prepared:

| | |
|---|---|
| | 5A |
| 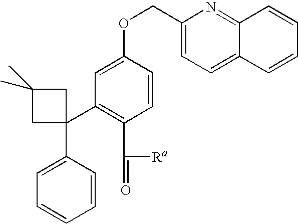 | |
| | 5B |
| 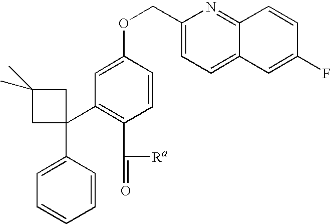 | |

| Ex. #5A | Ex. #5B | R^{a} |
|---|---|---|
| a | a | —N(H)Me |
| b | b | —NMe₂ |
| c | c | —N(H)Et |
| d | d | —NEt₂ |
| e | e | —N(H)Pr |
| f | f | —N(H)iPr |
| g | g | —N(H)cyclopropyl |
| h | h | —N(Me)Et |
| i | i | 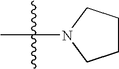 |
| j | j | 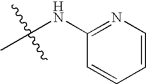 |
| k | k | 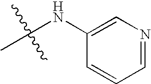 |
| l | l | 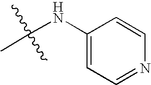 |
| m | m | 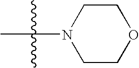 |
| n | n | 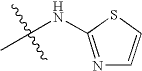 |
| o | o | 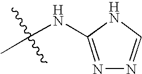 |
| — | p | -OMe |
| — | q | —OH |

In the examples shown above, the quinoline or 6-fluoroquinoline groups can also be replaced with 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 6

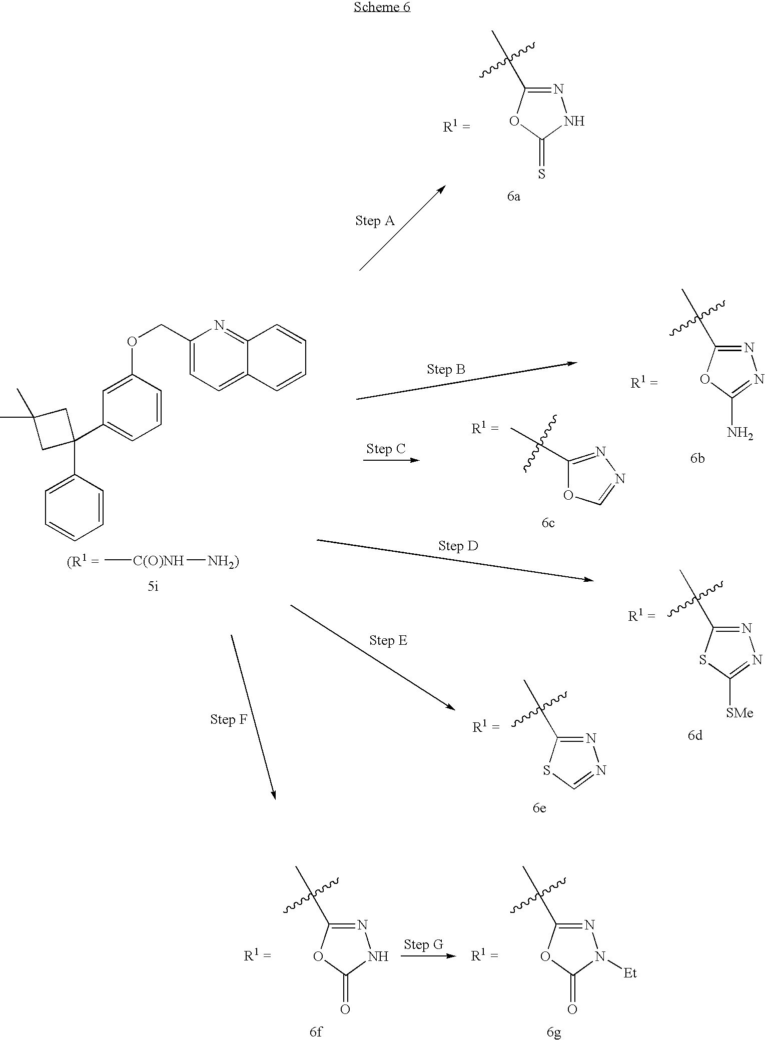

### Step A: Preparation of 5-[2-(3,3-dimethyl-1-phenylcyclobutyl)-4-(quinolin-2-ylmethoxy)phenyl]1,3,4-oxadiazole-2(3H)-thione (6a)

Thiophosgene (1.2 equiv.) is added dropwise via syringe to a stirred solution of 5i (1 equiv.) in THF at −78° C. After the reaction is deemed complete, the reaction mixture is poured into sat. aq. sodium bicarbonate and extracted three times with DCM. The combined organic extracts are washed with brine, dried (Na₂SO₄) and concentrated in vacuo. Purification of the crude residue by either flash chromatography on silica gel or preparative reversed phase HPLC affords the title compound 6a.

### Step B: Preparation of 5-[2-(3,3-dimethyl-1-phenylcyclobutyl)-4-(quinolin-2-ylmethoxy)phenyl]-1,3,4-oxadiazol-2-amine (6b)

Aq. sodium bicarbonate (1.2 equiv of a 0.5 N solution in water) is added dropwise via syringe to a stirred solution of 5i (1 equiv.) in dioxane at room temperature. A solution of cyanogen bromide (1.1 equiv.) in dioxane is then added, and the reaction mixture is aged at ambient temperature until the reaction is deemed complete. The reaction mixture is poured into sat aq. sodium bicarbonate and extracted three times with DCM. The combined organic extracts are washed with brine, dried (Na₂SO₄) and concentrated in vacuo. Purification of the crude residue by either flash chromatography on silica gel or preparative reversed phase HPLC affords the title compound 6b.

### Step C: Preparation of 2-{[3-(3,3-dimethyl-1-phenylcyclobutyl)-4-(1,34-oxadiazol-2-yl)phenoxy]methyl}quinoline (6c)

A solution of 5i.HCl salt (1 equiv.) and p-TSA (catalytic amount) in triethylorthoformate is aged at room temperature until the reaction is deemed complete. The reaction mixture is poured into sat. aq. sodium bicarbonate and extracted three times with EtOAc. The combined organic extracts are washed with brine, dried (Na₂SO₄) and concentrated in vacuo. Purification of the crude residue by either flash chromatography on silica gel or preparative reversed phase HPLC affords the title compound 6c.

### Step D: Preparation of 2-({3-(3,3-dimethyl-1-phenylcyclobutyl)-4-[5-(methylthio)-1,3,4-thiadiazol-2-yl]phenoxy}methyl)quinoline (6d)

Potassium hydroxide (0.95 equiv.) is added to a solution of 5i (1 equiv.) and carbon disulfide (2.1 equiv.) in MeOH at 0° C. After approximately 2 h, the reaction mixture is warmed to room temperature and aged for a further 4 h. Iodomethane (1 equiv.) is added and the resulting mixture is aged until the reaction is deemed complete. The reaction mixture is poured into water and extracted three times with DCM. The combined organic extracts are washed with sat. aq. sodium bicarbonate, brine, dried (Na₂SO₄) and concentrated in vacuo. The crude intermediary hydrazinecarbodithioate is dissolved in toluene, and p-toluenesulfonic acid (1.1 equiv.) is added. The resulting mixture is heated at reflux until the reaction is deemed complete. After cooling to room temperature, the reaction mixture is poured into water and extracted three times with DCM. The combined organic extracts are washed with sat. aq. sodium bicarbonate, brine, dried (Na₂SO₄) and concentrated in vacuo. Purification of the crude residue by either flash chromatography on silica gel or preparative reversed phase HPLC affords the title compound 6d.

### Step E: Preparation of 2-{[3-(3,3-dimethyl-1-phenylcyclobutyl)-4-(1,34-thiadiazol-2-yl)phenoxy]methyl}quinoline (6e)

A solution of 5i (1 equiv.) in formic acid (96%, excess) is aged at room temperature until the starting material is consumed. The reaction mixture is concentrated in vacuo, and the residue is partitioned between DCM and sat. aq. sodium bicarbonate. The organic phase is separated and the aq. phase is re-extracted twice with DCM. The combined organic extracts are washed with brine, dried (Na₂SO₄) and concentrated in vacuo. The crude intermediary formylhydrazide is treated with phosphorous pentasulfide (1.1 equiv.) in dioxane. The resulting mixture is heated at approximately 50° C. until the reaction is deemed complete. After cooling to room temperature, the reaction mixture is poured into 1 N aq. sodium hydroxide and extracted three times with DCM. The combined organic extracts are washed with brine, dried (Na₂SO₄) and concentrated in vacuo. Purification of the crude residue by either flash chromatography on silica gel or preparative reversed phase HPLC affords the title compound 6e.

### Step F: Preparation of 5-[2-(3,3-dimethyl-1-phenylcyclobutyl)-4-(quinolin-2-ylmethoxy)phenyl]-1,3,4-oxadiazol-2(3H)-one (6f)

Phosgene (174 mL of a 20% solution in toluene, 0.33 mmol) was added dropwise via syringe to a stirred solution of crude 5i (0.18 mmol) in DCM (2.2 mL) at −78° C. After approximately 50 min, the reaction mixture was poured into sat. aq. sodium bicarbonate and extracted three times with DCM. The combined organic extracts were washed with brine, dried (MgSO₄) and concentrated in vacuo. Purification of the crude residue by flash chromatography on silica gel (gradient elution; 0-50% EtOAc/hexanes as eluent) furnished the title compound 6f, m/z (ES) 478 (MH)⁺.

### Step G: Preparation of 5-[2-(3,3-dimethyl-1-phenylcyclobutyl-4-(quinolin-2-ylmethoxy)phenyl]-3-ethyl-1,3,4-oxadiazol-2(3H)-one (6g)

Sodium hydride (2 equiv.) is added to a solution of 6f (1 equiv.) and iodoethane (1.5 equiv.) in DMF at 0° C. After the reaction is deemed complete, the reaction mixture is quenched with sat. aq. ammonium chloride, poured into sat. aq. sodium bicarbonate and extracted three times with EtOAc. The combined organics are washed successively with water and brine, dried (Na₂SO₄) and concentrated in vacuo. The crude residue can be purified by flash chromatography on silica gel to afford the title compound 6g.

Following procedures similar to that described above for Examples 6a-g the following compounds can be prepared (*compounds can be either racemic or chiral):

| | |
|---|---|
| | 6A |
| 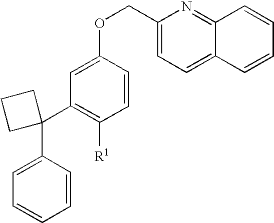 | |
| | 6B |
| 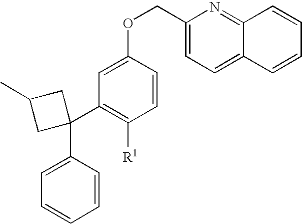 | |
| | 6C |
| 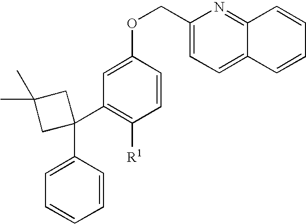 | |
| | 6D |
| 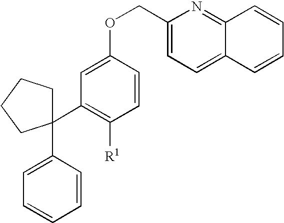 | |
| | 6E |
| 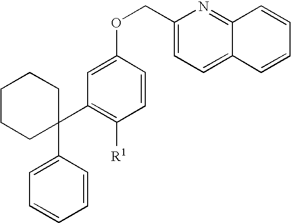 | |

| Ex. #6A | Ex. #6B* | Ex. #6C | Ex. #6D | Ex. #6E | R¹ |
|---|---|---|---|---|---|
| a | a | — | a | a | 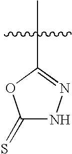 |
| b | b | — | b | b | 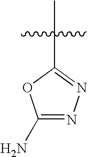 |
| c | c | — | c | c | 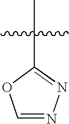 |
| d | d | — | d | d | 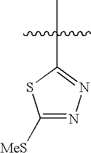 |
| e | e | — | e | e | 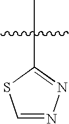 |
| f | f | — | f | f | 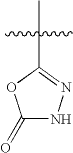 |
| g | g | — | g | g | 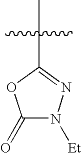 |
| h | h | h | h | h | 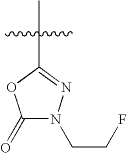 |

In the examples shown above, the quinoline group can also be replaced with 6-fluoroquinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 7

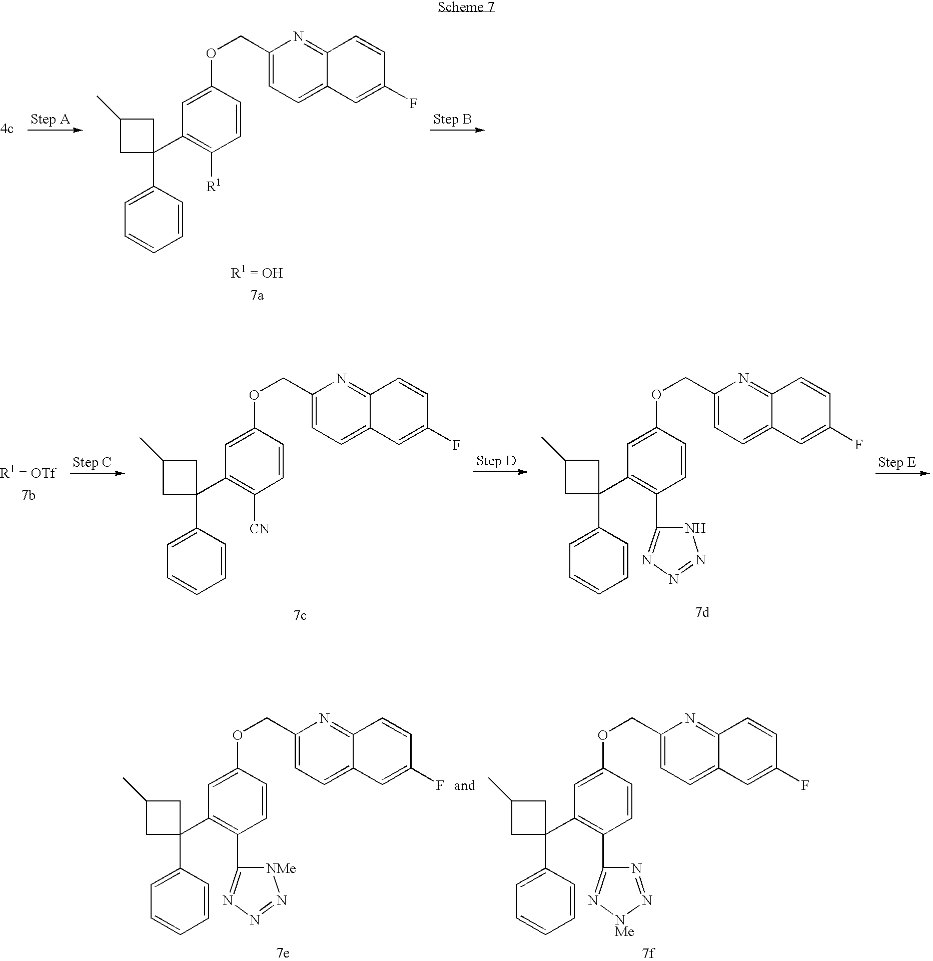

### Steps A: Preparation of 4-[(6-fluoroquinolin-2-yl)methoxy]-2(3-methyl-1-phenylcyclo-butyl)phenol (7a)

Compound 7a can be prepared from 4c following the procedure outlined in Scheme 4, step D.

### Step B: Preparation of 4-[(6-fluoroquinolin-2-yl)methoxy]-2-(3-methyl-1-phenylcyclobutyl)-phenyltrifluoromethanesulfonate (7b)

Compound 7b can be prepared from 7a following the procedure outlined in Scheme 4, step E.

### Step C: Preparation of 4-[(6-fluoroquinolin-2-yl)methoxy]-2-(3-methyl-1-phenylcyclobutyl)-benzonitrile (7c)

Zinc cyanide (1 equiv.), tris(dibenzylideneacetone)dipalladium(0) (0.2 equiv.), and dppf (0.5 equiv.) are added successively to a stirred solution of 4e (1 equiv.) in NMP. After degassing the resulting mixture with a gentle stream of dry nitrogen for approximately 10 min, the reaction mixture is heated to 140° C. After the reaction is deemed complete, the reaction mixture is cooled to room temperature, and filtered through a short column of silica gel eluting with EtOAc. The filtrate is washed twice with water, brine, dried (MgSO₄) and concentrated in vacuo. The crude residue can be purified by flash chromatography on silica gel to give the title compound 7c.

### Step D: Preparation of 6-fluoro-2-{[3-(3-methyl-1-phenylcyclobutyl)-4-(1H-tetrazol-5-yl)phenoxy]methyl}quinoline (7d)

Azidotrimethyltin (4 equiv.) is added to a stirred solution of 7c (1 equiv.) in toluene at room temperature and the resulting mixture is heated to 140° C. After the reaction is deemed complete (typically 2-3 d), the reaction mixture is cooled to room temperature, and the volatiles are removed in vacuo. The residue is taken up in cold hydrogen chloride/MeOH (sat. solution) and stirred for approximately 30 min at room temperature. The reaction mixture is concentrated in vacuo, and the crude residue can be purified by flash chromatography on silica gel to furnish the title compound 7d.

### Step E: Preparation of 6-fluoro-2-{[3-(3-methyl-1-phenylcyclobutyl)-4-(1-methyl-1H-tetrazol-5-yl)phenoxy]methyl}quinoline (7e) and 6-fluoro-2-{[3-(3-methyl-1-phenylcyclobutyl)-4-(2-methyl-2H-tetrazol-5-yl)phenoxy]methyl}quinoline (7f)

Freshly ground anhydrous potassium carbonate (4 equiv.) is added to a stirred solution of 7d (1 equiv.) in DMF at room temperature. After 1 h, methyl iodide (2 equiv.) is added via syringe. After the reaction is deemed complete, the reaction mixture is poured into water and extracted three times with EtOAc. The combined organic extracts are washed repeatedly with water, brine, dried (MgSO₄) and concentrated. The crude residue can be purified by flash chromatography on silica gel to afford the title compounds 7e and 7f.

7e and 7f can be individually resolved into their enantiomeric components using chiral HPLC techniques.

Following procedures similar to that described above for Examples 7e and 7f, the following compounds can be prepared (*compounds can be either racemic or chiral):

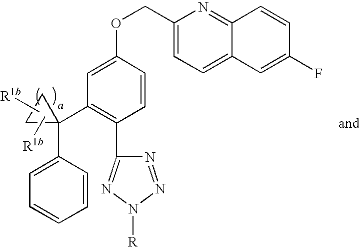

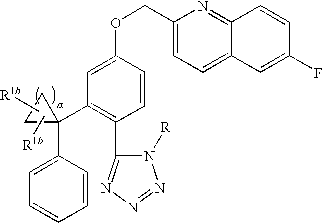

wherein

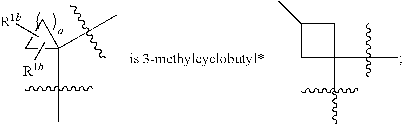

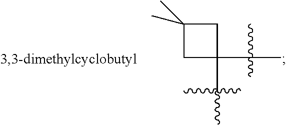

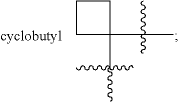

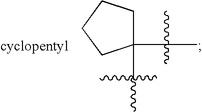

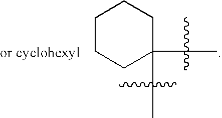

| Ex. #7 | 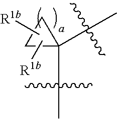 | R |
|---|---|---|
| g* | 3-methylcyclobutyl | Et |
| h* | 3-methylcyclobutyl | iso-Pr |
| i* | 3-methylcyclobutyl | CHF₂ |
| j* | 3-methylcyclobutyl | CH₂CH₂F |
| k | 3,3-dimethylcyclobutyl | Me |
| l | 3,3-dimethylcyclobutyl | Et |
| m | 3,3-dimethylcyclobutyl | iso-Pr |
| n | 3,3-dimethylcyclobutyl | CHF₂ |
| o | 3,3-dimethylcyclobutyl | CH₂CH₂F |
| p | cyclobutyl | Me |
| q | cyclobutyl | Et |
| r | cyclobutyl | iso-Pr |
| s | cyclobutyl | CHF₂ |
| t | cyolobutyl | CH₂CH₂F |
| u | cyclopentyl | Me |
| v | cyclopentyl | Et |
| w | cyclopentyl | iso-Pr |
| x | cyclopentyl | CHF₂ |
| y | cyclopentyl | CH₂CH₂F |
| z | cyclohexyl | Me |
| aa | cyclohexyl | Et |
| ab | cyclohexyl | iso-Pr |
| ac | cyclohexyl | CHF₂ |
| ad | cyclohexyl | CH₂CH₂F |

In the examples shown above, the 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 8

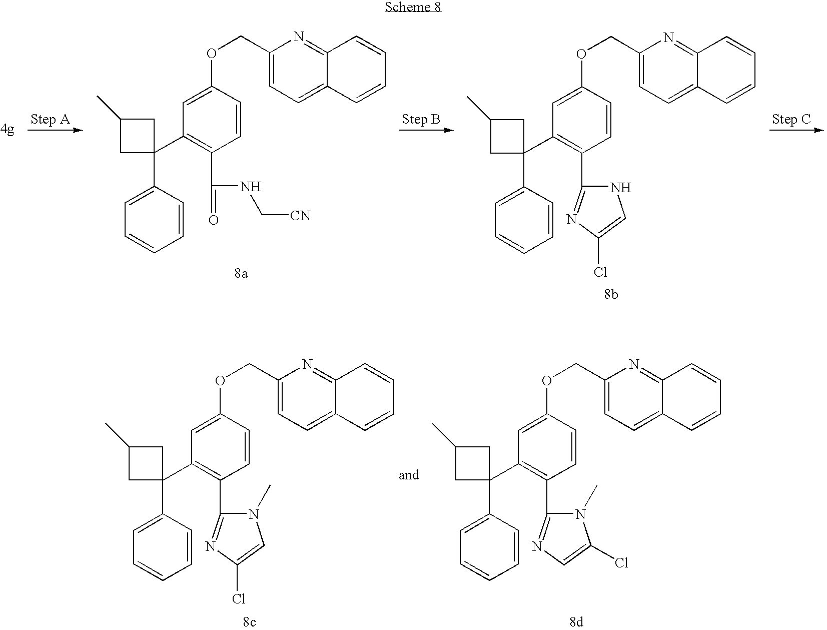

### Step A: Preparation of N-(cyanomethyl)-2-(3-methyl-1-phenylcyclobutyl)-4-(quinolin-2-ylmethoxy)benzamide (8a)

Aminoacetonitrile hydrochloride (1.05 equiv.), triethylamine (2.5 equiv.), HATU (1.05 equiv.), and DMAP (0.20 equiv.) are added successively to a stirred solution of 4g (1 equiv.) in DCM/DMF (9:1) at room temperature. After the reaction is deemed complete, the reaction mixture is poured into water and extracted three times with EtOAc. The combined organic extracts are washed twice with 5% citric acid, three times with water, brine, dried (MgSO₄) and concentrated in vacuo. The crude residue can be purified by flash chromatography on silica gel to afford the title compound 8a.

### Step B: Preparation of 2-{[4-(4-chloro-1f-imidazol-2-yl)-3-(3-methyl-1-phenylcyclobutyl)phenoxy]methyl}quinoline (8b)

Triphenylphosphine (2.4 equiv.) is added to a stirred solution of 8a (1 equiv.) in acetonitrile at room temperature. Upon dissolution, carbon tetrachloride (2.4 equiv.) is added dropwise via syringe. The resulting mixture is heated to approximately 50° C. and stirred until the reaction is deemed complete. After cooling to room temperature, the volatiles are removed in vacuo. The residue is taken up in DCM, then sat. aq. sodium bicarbonate is added, and the resulting biphasic mixture is stirred vigorously for approximately 15 min at room temperature. The organic phase is separated and the aq. phase is extracted twice with EtOAc. The combined organic extracts are washed with water, brine, dried (MgSO₄) and concentrated in vacuo. The crude residue can be purified by flash chromatography on silica gel to furnish the title compound 8b.

### Step C: Preparation of 2-{[4-(4-chloro-1-ethyl-1H-imidazol-2-yl)-3-(3-methyl-1-phenylcyclobutyl)phenoxy]methyl}quinoline (8c) and 2-{[4-(5-chloro-1-methyl-1H-imidazol-2-yl)-3-(3-methyl-1-phenylcyclobutyl)phenoxy]methyl}quinoline (8d)

Freshly ground anhydrous potassium carbonate (1.5 equiv.) is added to a stirred solution of 8b (1 equiv.) in DMF at room temperature. After 1 h, methyl iodide (1.5 equiv.) is added via syringe and the resulting mixture is stirred at room temperature until the reaction is deemed complete. The reaction mixture is poured into water and extracted three times with EtOAc. The combined organic extracts are washed three times with water, brine, dried (MgSO₄) and concentrated in vacuo. The crude residue can be purified by flash chromatography on silica gel to provide the title compounds 8c and 8d, 8c and 8d can be individually resolved into their enantiomeric components using chiral HPLC techniques.

Following procedures similar to that described above for Examples 8a-d, the following compounds can be prepared (*compounds can be either racemic or chiral):

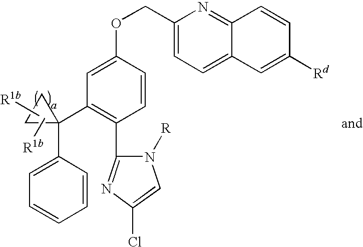

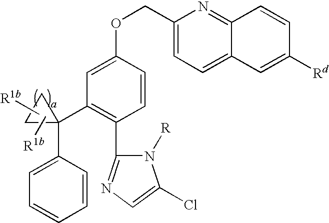

wherein

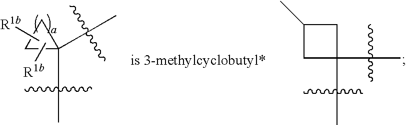

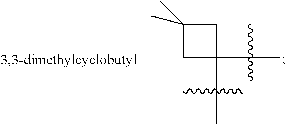

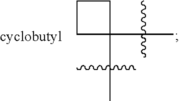

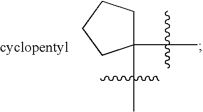

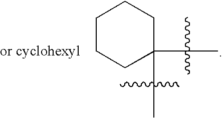

| Ex. #8 | 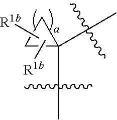 | R | R^{d} |
|---|---|---|---|
| e* | 3-methylcyclobutyl | H | F |
| f* | 3-methylcyclobutyl | Me | F |
| g | 3,3-dimethylcyclobutyl | H | H |
| h | 3,3-dimethylcyclobutyl | H | F |
| i | 3,3-dimethylcyclobutyl | Me | F |
| l | cyclobutyl | H | H |
| m | cyclobutyl | H | F |
| n | cyclobutyl | Me | F |
| q | cyclopentyl | H | H |
| r | cyclopentyl | H | F |
| s | cyclopentyl | Me | F |
| v | cyclohexyl | H | H |
| w | cyclohexyl | H | F |
| z | cyclohexyl | Me | F |

In the examples shown above, the quinoline or 6-fluoroquinoline groups can also be replaced with 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 9

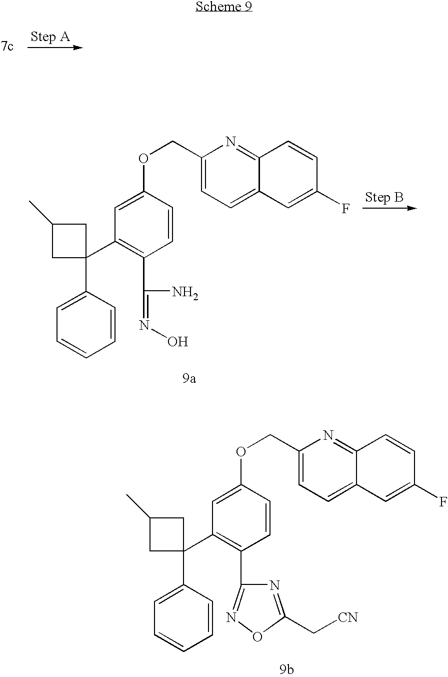

### Step A: Preparation of 4-[(6-fluoroquinolin-2-yl)methoxy]-N′-hydroxy-2-(3-methyl-1-phenylcyclobutyl)benzenecarboximidamide (9a)

A thick-walled tube is charged with a solution of 7c (1 equiv.) in anhydrous EtOH (0.3 M). Hydroxylamine (5 equiv. of a 50% weight solution in water) is added and the resulting mixture is sealed and stirred at 120° C. until the reaction is deemed complete. After cooling to room temperature, the reaction mixture is concentrated in vacuo and the crude product is purified by flash chromatography on silica gel to provide the title compound 9a.

### Step B: Preparation of {3-[4-[(6-fluoroquinolin-2-yl)methoxy]-2-(3-methyl-1-phenylcyclobutyl)-phenyl]-1,2,4-oxadiazol-5-yl}acetonitrile (9b)

A solution of cyanoacetic acid (5 equiv.) and dicyclohexylcarbodiimide (2.5 equiv.) in DCM (1.0 M concentration in cyanoacetic acid) is stirred at room temperature until the reaction is deemed complete. The reaction mixture is concentrated in vacuo, and the residue is taken up in anhydrous ether. The precipitate (dicyclohexylurea) is removed via filtration, and the filtrate is concentrated to dryness and then dissolved in anhydrous pyridine (0.5 M in 9a). To this mixture is added 9a (1 equiv.) and the resulting mixture is heated at 140° C. until all of the starting material has been consumed. After cooling to room temperature, the reaction mixture is concentrated in vacuo and the crude product is purified by flash chromatography on silica gel to provide the title compound 9b.

Following procedures similar to that described above for Example 9b, the following compounds have been prepared (*compounds can be either racemic or chiral):

| | |
|---|---|
| | 9A |
| 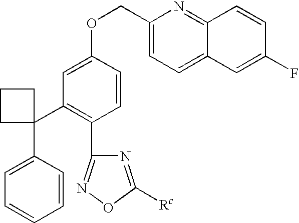 | |
| | 9B |
| 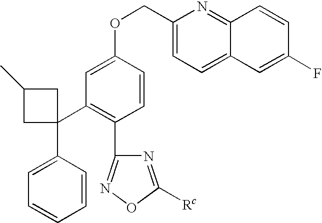 | |
| | 9C |
| 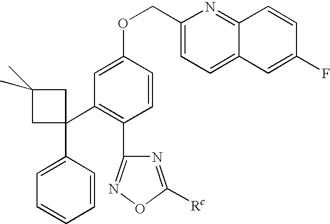 | |
| | 9D |
| 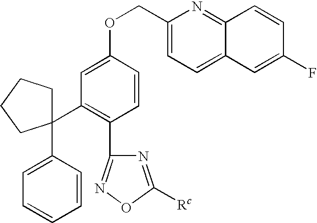 | |
| | 9E |
| 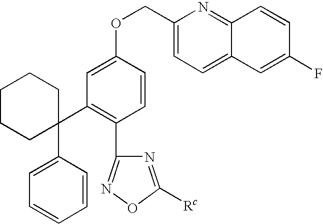 | |

| Ex. #9A | Ex. #9B* | Ex. #9C | Ex. #9D | Ex. #9E | R^{c} |
|---|---|---|---|---|---|
| a | a | a | a | a | Me |
| b | b | b | b | b | Et |
| c | c | c | c | c | iso-Pr |
| d | d | d | d | d | CH₂F |
| e | e | e | e | e | CHF₂ |
| f¹ | f¹ | f¹ | f¹ | f¹ | CH₂OH |
| g¹ | g¹ | g¹ | g¹ | g¹ | 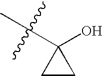 |
| h¹ | h¹ | h¹ | h¹ | h¹ | 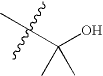 |
| i | — | i | i | i | CH₂CN |

¹The alcohol functionality was masked as a benzyl ether in Step B. Deprotection of the benzyl ether was achieved with methanesulfonic acid (according to J. Am. Chem. Soc., 1996, 118, 4560) to provide the desired alcohol product.

In the examples shown above, the 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 10

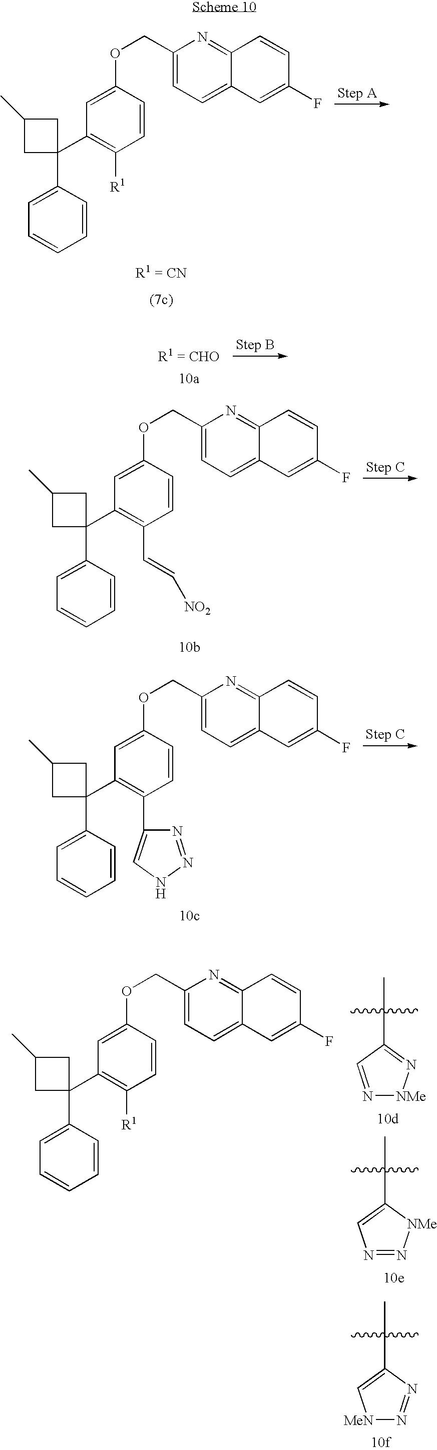

### Step A: Preparation of 4-[(6-fluoroquinolin-2-yl)methoxy]-2-(3-methyl-1-phenylcyclobutyl)-benzaldehyde (10a)

DIBAL-H (4 equiv.) is added to a stirred solution of 7c (1 equiv.) in DCM (0.1 M) at −78° C. After the reaction is deemed complete, the reaction mixture is quenched with wet silica gel (excess) and the resulting mixture is stirred vigorously for approximately 30 min. The slurry is filtered and the residue washed with EtOAc. The filtrate is washed with water, brine, dried (Na₂SO₄) and concentrated in vacuo. Purification of the crude residue by flash chromatography affords the title compound 10a.

### Step B: Preparation of 6-fluoro-2-({3-(3-methyl-1 penylcyclobutyl)-4-[(E)-2-nitrovinyl]phenoxy}methyl)quinoline (10b)

A microwave tube is charged with nitromethane (5 equiv.), ammonium acetate (0.25 equiv.) and 10a (1 equiv.). The resulting mixture is irradiated in a microwave apparatus (300 W) at 100° C. until the reaction is deemed complete. After cooling to room temperature, the reaction mixture is filtered, and the residue washed copiously with EtOAc. The filtrate is evaporated in vacuo, and the residue is purified by flash chromatography to provide the title compound 10b.

### Step C: Preparation of 6-fluoro-2-{[3-(3n-methyl-1-phenylcyclobutyl)-4-(1H-1,2,3-triazol-4-yl)phenoxy]methyl}quinoline (10c)

Sodium azide (3 equiv.) is added to a stirred solution of 10b (1 equiv.) in DMSO (0.8 M) at room temperature and the resulting mixture is stirred at 50° C. until the reaction is deemed complete. The reaction mixture is cooled to room temperature, poured into water, and extracted three times with EtOAc. The combined organic extracts are washed three times with water, brine, dried (MgSO₄), and concentrated in vacuo. Purification of the crude residue by flash chromatography on silica gel affords the title compound 10c.

### Step D: Preparation of 6-fluoro-2-{[3-(3-methyl-1-phenylcyclobutyl)-4-(2-methyl-2H-1,2,3-triazol-4-yl)phenoxy]methyl}quinoline (10d), 6-fluoro-2-{[3-(3-methyl-1-phenylcyclobutyl)-4-(1-methyl-1H-1,2,3-triazol-5-yl)phenoxy]methyl}quinoline (10e), and 6-fluoro-2-{[3-(3-methyl-1-phenylcyclobutyl)-4-(1-methyl-1H-1,2,3-triazol-4-yl)phenoxy]methyl}quinoline (10f)

Freshly ground anhydrous potassium carbonate (1.7 equiv.) is added to a stirred solution of 10c (1 equiv.) in DMF (0.2 M) at room temperature. After approximately 1 h, methyl iodide (1.3 equiv.) is added via syringe. After the reaction is deemed complete, the reaction mixture is poured into water, adjusted to pH 5 with aq. citric acid, and extracted three times with EtOAc. The combined organic extracts are washed repeatedly with water, brine, dried (MgSO₄) and concentrated in vacuo. Purification of the crude residue by flash chromatography provides a separable mixture of 10d, 10e and 10f.

NB. In instances where inseparable mixtures are obtained initially, and individual compound characterization is desired, other chromatographic techniques can be employed to achieve resolution, such as reversed-phase HPLC, normal phase chiral HPLC, normal phase chiral SFC etc. Structural assignments are confirmed, typically by using a combination of spectroscopic techniques including for instance ¹H-NMR, ¹H-nOe etc.

Following procedures similar to that described above for Example 10c-f, the following compounds have been prepared (*compounds can be either racemic or chiral):

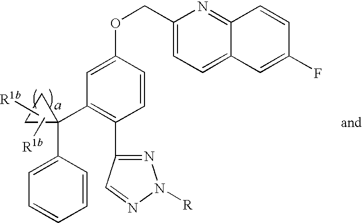

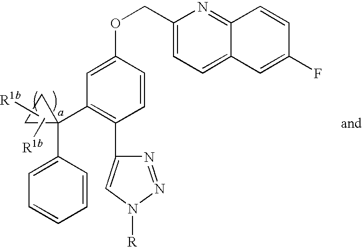

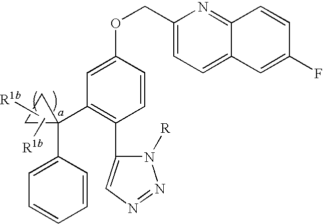

wherein

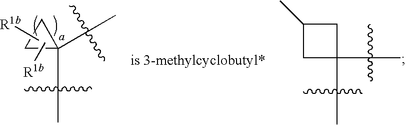

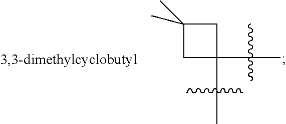

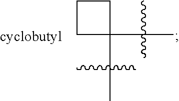

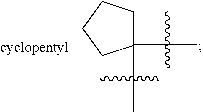

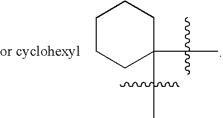

| Ex. #10 | 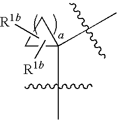 | R |
|---|---|---|
| g* | 3-methylcyclobutyl | Et |
| h* | 3-methylcyclobutyl | iso-Pr |
| i* | 3-methylcyclobutyl | CH₂CN |
| j* | 3-methylcyclobutyl | CH₂CH₂CN |
| k* | 3-methylcyclobutyl | CH₂CH₂F |
| l* | 3-methylcyclobutyl | CH₂CH₂OH |
| m* | 3-methylcyclobutyl | 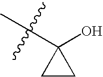 |
| n* | 3-methylcyclobutyl | 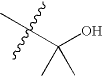 |
| o | 3,3-dimethylcyclobutyl | H |
| p | 3,3-dimethylcyclobutyl | Me |
| q | 3,3-dimethylcyclobutyl | Et |
| r | 3,3-dimethylcyclobutyl | iso-Pr |
| s | 3,3-dimethylcyclobutyl | CH₂CN |
| t | 3,3-dimethylcyclobutyl | CH₂CH₂CN |
| u | 3,3-dimethylcyclobutyl | CH₂CH₂F |
| v | 3,3-dimethylcyclobutyl | CH₂CH₂OH |
| w | 3,3-dimethylcyclobutyl | 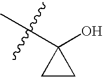 |
| x | 3,3-dimethylcyclobutyl | 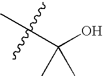 |
| y | cyclobutyl | H |
| z | cyclobutyl | Me |
| aa | cyclobutyl | Et |
| ab | cyclobutyl | iso-Pr |
| ac | cyclobutyl | CH₂CN |
| ad | cyclobutyl | CH₂CH₂CN |
| ae | cyclobutyl | CH₂CH₂F |
| af | cyclobutyl | CH₂CH₂OH |
| ag | cyclobutyl | 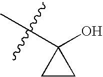 |
| ah | cyclobutyl | 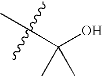 |
| ai | cyclopentyl | H |
| aj | cyclopentyl | Me |
| ak | cyclopentyl | Et |
| al | cyclopentyl | iso-Pr |
| am | cyclopentyl | CH₂CN |
| an | cyclopentyl | CH₂CH₂CN |
| ao | cyclopentyl | CH₂CH₂F |
| ap | cyclopentyl | CH₂CH₂OH |
| aq | cyclopentyl | 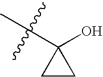 |
| ar | cyclopentyl | 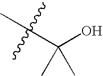 |
| as | cyclohexyl | H |
| at | cyclohexyl | Me |
| au | cyclohexyl | Et |
| av | cyclohexyl | iso-Pr |
| aw | cyclohexyl | CH₂CN |
| ax | cyclohexyl | CH₂CH₂CN |
| ay | cyclohexyl | CH₂CH₂F |
| az | cyclohexyl | CH₂CH₂OH |
| ba | cyclohexyl | 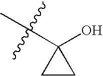 |
| bb | cyclohexyl | 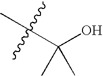 |

In the examples shown above, the 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 11

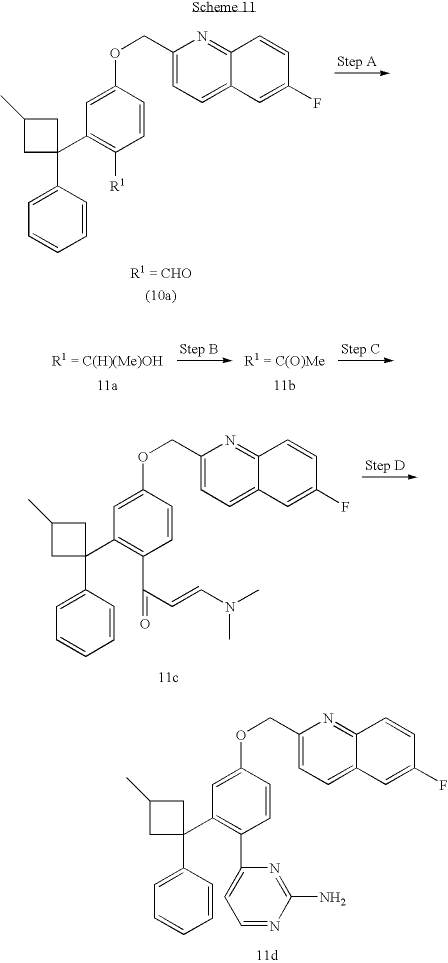

### Step A: Preparation of 1-[4-[(6-fluoroquinolin-2-yl)methoxy]-2-(3-methyl-1-phenylcyclobutyl)phenyl]ethanol (11a)

Methyl magnesium bromide (1.5 equiv.) is added to a stirred solution of 10a (1 equiv.) in THF (0.1 M) at 0° C. After the reaction is deemed complete, the reaction mixture is quenched with aq. ammonium chloride and extracted three times with EtOAc. The combined organic extracts are washed with water, brine, dried (MgSO₄) and concentrated in vacuo. Purification of the crude residue by flash chromatography affords the title compound 11a.

### Step B: Preparation of 1-[4-[(6-fluoroquinolin-2-yl)methoxy]-2-(3-methyl-1-phenylcyclobutyl)phenyl]ethanone (11b)

Manganese(IV) oxide (10 equiv.), followed by Celite® (excess; ˜equal weight to manganese(IV) oxide) are added to a stirred solution of 11a (1 equiv.) in toluene (0.3 M) at room temperature. The resulting mixture is heated to approximately 100° C. and stirred until the reaction is deemed complete. After cooling to room temperature, the reaction mixture is filtered and the residue washed copiously with EtOAc. The filtrate is concentrated in vacuo and the crude residue is purified by flash chromatography to provide the title compound 11b.

### Step C: Preparation of (2E)-3-(dimethylamino)-1-[4-[(6-fluoroquinolin-2-yl methoxy]-2-(3-methyl-1-phenylcyclobutyl)phenyl]prop-2-en-1-one (11c)

A thick-walled pressure tube is charged with 11b and N,N-dimethylformamide diethyl acetal (excess), The resulting mixture is irradiated in a microwave apparatus (300 W) at 120° C. until the reaction is deemed complete. After cooling to room temperature, the reaction mixture is concentrated in vacuo and the crude residue is purified by flash chromatography to afford the title compound 11c.

### Step D: Preparation of 4-[4-[(6-fluoroquinolin-2-yl)methoxy]-2-(3-methyl-1-phenylcyclobutyl)phenyl]pyrimidin-2-amine (11d)

Guanidine hydrochloride (2 equiv.), followed by sodium methoxide (2.4 equiv.) are added to a stirred solution of 11c (1 equiv.) in EtOH (0.1 M) at room temperature. The resulting mixture is sealed, and stirred at 78° C. until the reaction is deemed complete. After cooling to room temperature, the reaction mixture is concentrated in vacuo and the residue partitioned between EtOAc and water. The organic phase is separated, and the aq. layer is extracted with EtOAc. The combined organic extracts are washed with brine, dried (MgSO₄) and concentrated in vacuo. The crude residue is purified by either flash chromatography or preparative TLC to provide the title compound 11d.

Following procedures similar to above for Example 11d the following compounds (*compounds can be either racemic or chiral) can be prepared.

| | |
|---|---|
| | 11A |
| 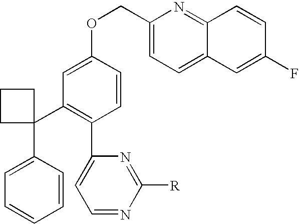 | |
| | 11B |
| 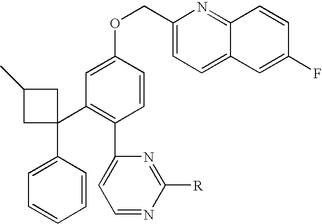 | |
| | 11C |
| 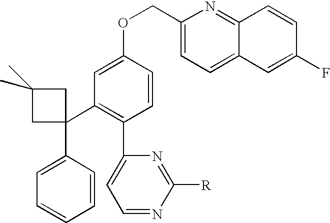 | |
| | 11D |
| 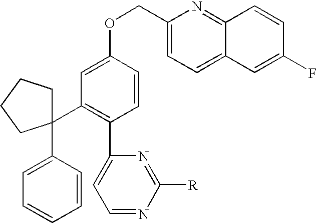 | |
| | 11E |
| 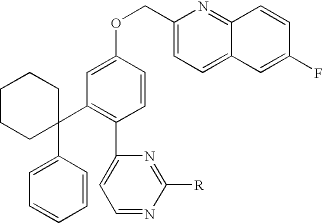 | |

| Ex. #11A | Ex. #11B* | Ex. #11C | Ex. #11D | Ex. #11E | R |
|---|---|---|---|---|---|
| a | a | a | a | a | Me |
| b | b | b | b | b | Et |
| c | c | c | c | c | iso-Pr |
| c | c | c | c | c | cyclopropyl |

In the examples shown above, the 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 12

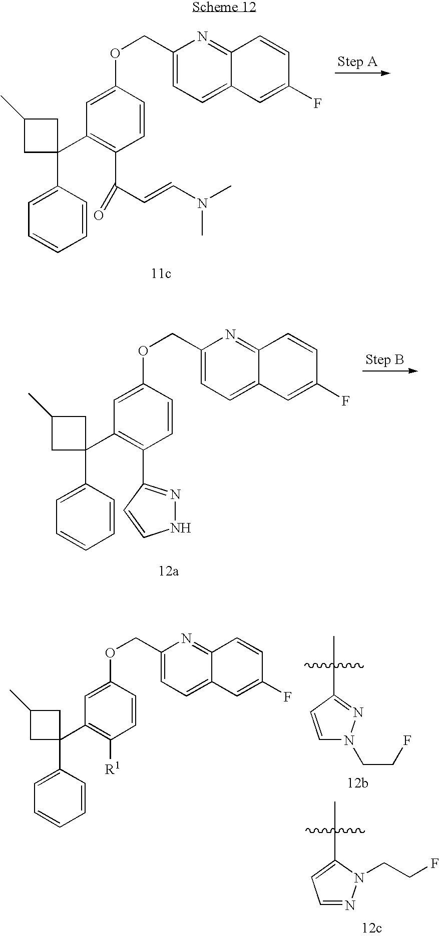

### Step A: Preparation of 6-fluoro-2-{[3-(3-methyl-1-phenylcyclobutyl)-4-(1H-pyrazol-3-yl)phenoxy]methyl}quinoline (12a)

Anhydrous hydrazine (excess) is added to a stirred solution of 11c in EtOH (0.1 M) and the resulting mixture heated in an oil bath at 110° C. until the reaction is deemed complete. After cooling to room temperature, the volatiles are removed in vacuo. The crude residue is purified by either flash chromatography or preparative TLC to provide the title compound 12a.

### Step B: Preparation of 6-fluoro-2-{[4-[1-(2-fluoroethyl)-1H-pyrazol-3-yl]-3-(3-methyl-1-phenylcyclobutyl)phenoxy]methyl}quinoline (12b) and 6-fluoro-2-{[4-[1-(2-fluoroethyl)-1H-pyrazol-5-yl]-3-(3-methyl-1-phenylcyclobutyl)phenoxy]methyl}-quinoline (12c)

Sodium hydride (1.4 equiv.) is added to a stirred solution of 12a (1 equiv.) in DMF (0.05 M) at 0° C. After 10 min, 1-bromo-2-fluoroethane (1.3 equiv.) is added via syringe. The resulting mixture is warmed to room temperature and aged until the reaction is deemed complete. The reaction mixture is quenched with sat. aq. ammonium chloride and then extracted three times with EtOAc. The combined organic extracts are washed with water, brine, dried (MgSO₄) and concentrated in vacuo. The residue is purified by either flash chromatography or preparative TLC to afford a separable mixture of the title compounds 12b and 12c.

Following procedures similar to above for Example 12a-c the following compounds (*compounds cab be either racemic or chiral) can be prepared.

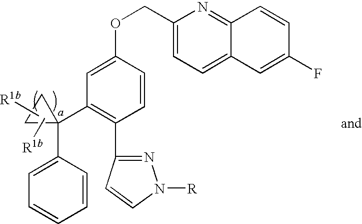

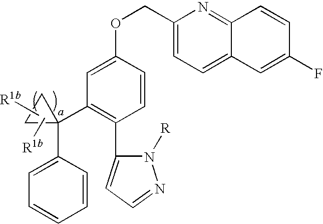

wherein

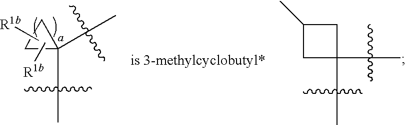

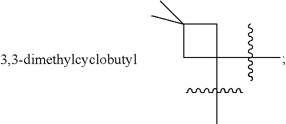

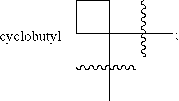

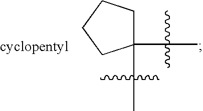

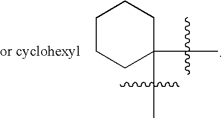

| Ex. #12 | 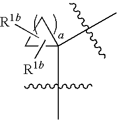 | R |
|---|---|---|
| d* | 3-methylcyclobutyl | Me |
| e* | 3-methylcyclobutyl | Et |
| f* | 3-methylcyclobutyl | iso-Pr |
| g | 3,3-dimethylcyclobutyl | Me |
| h | 3,3-dimethylcyclobutyl | Et |
| i | 3,3-dimethylcyclobutyl | iso-Pr |
| j | 3,3-dimethylcyclobutyl | CH₂CH₂F |
| l | cyclobutyl | Me |
| m | cyclobutyl | Et |
| n | cyclobutyl | iso-Pr |
| o | cyclobutyl | CH₂CH₂F |
| p | cyclopentyl | Me |
| q | cyclopentyl | Et |
| r | cyclopentyl | iso-Pr |
| s | cyclopentyl | CH₂CH₂F |
| t | cyclohexyl | Me |
| u | cyclohexyl | Et |
| v | cyclohexyl | iso-Pr |
| w | cyclohexyl | CH₂CH₂F |

In the examples shown above, the 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 13

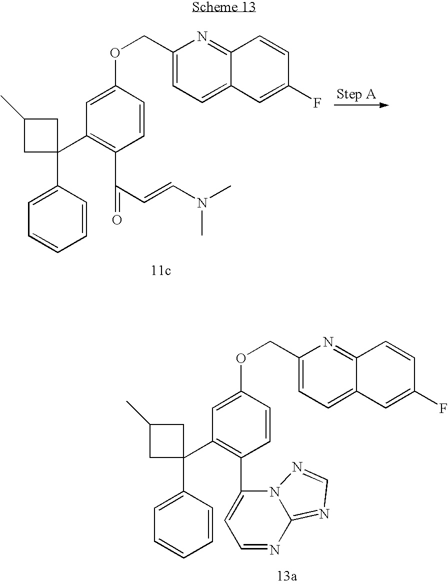

### Step A: Preparation of 6-fluoro-2-{[3-(3-methyl-1-phenylcyclobutyl)-4-[1,2,4]triazolor[1,5-a]pyrimidin-7-ylphenoxy]methyl}quinoline (13a)

1H-1,2,4-triazol-5-amine (2 equiv.) is added to a stirred solution of 11c (1 equiv.) in acetic acid (0.1 M) at room temperature. The resulting mixture is heated to 117° C. and stirred until the reaction is deemed complete. After cooling to room temperature, the volatiles are removed in vacuo and the residue is partitioned between EtOAc and sat. aq. sodium bicarbonate. The organic phase is separated and the aq. phase is extracted with EtOAc. The combined organic extracts are washed with water, brine, dried (MgSO₄) and concentrated in vacuo. The crude residue is purified by either flash chromatography or preparative TLC to afford 13a.

Following procedures similar to above for Example 13a the following compounds can be prepared, in which the depicted 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

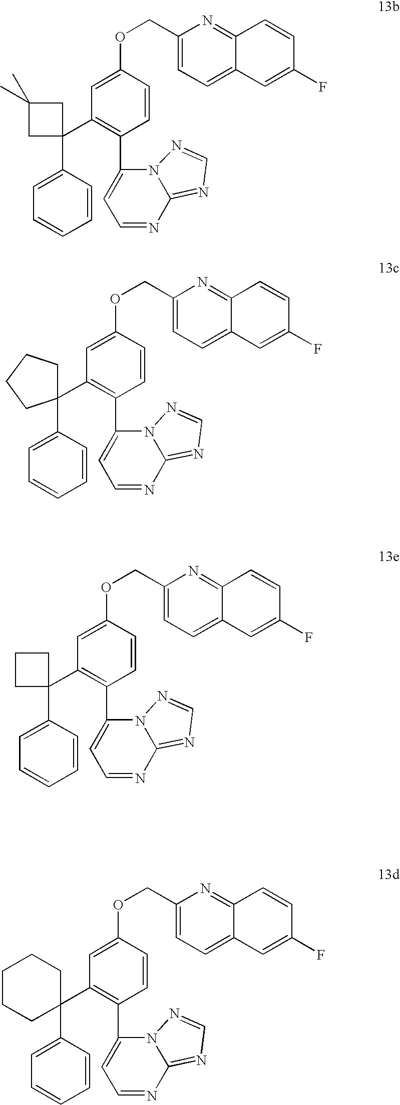

### Example 14

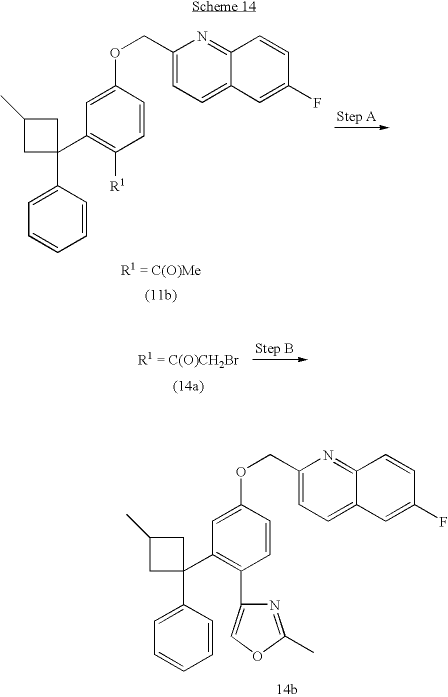

### Step A: Preparation of 2-bromo-1-[4-[(6-fluoroquinolin-2-yl)methoxy]-2-(3-methyl-1-phenylcyclobutyl)phenyl]ethanone (14a)

Pyrrolidinone hydrotribromide (1.1 equiv.) is added to a stirred solution of 11b (1 equiv.) in THF (0.5 M) at room temperature. The resulting mixture is warmed to 40° C. and aged until the reaction is deemed complete. After cooling to room temperature, the reaction mixture is poured into sat. aq. sodium bicarbonate and extracted three times with EtOAc. The combined organic extracts are washed with brine, dried (MgSO₄) and concentrated in vacuo. Purification of the crude residue by flash chromatography affords the title compound 14a.

### Step B: Preparation of 6-fluoro-2-{[4-(2-meth 1-1,3-oxazol-4-yl)-3-(3-methyl-1-phenylcyclobutyl)phenoxy]methyl}quinoline (14b)

A stirred mixture of acetamide (excess, >25 equiv.) and 14a (1 equiv.) is heated to 170° C. and aged until the reaction is deemed complete. After cooling to room temperature, the reaction mixture is diluted with water and extracted three times with EtOAc. The combined organic extracts are washed with sat. aq. sodium bicarbonate, water, brine, dried (MgSO₄) and concentrated in vacuo. Purification of the crude residue by preparative TLC or flash chromatography affords the title compound 14b.

Following procedures similar to that described above for Example 14b, the following compounds (*compounds can racemic or chiral) can be prepared:

| | |
|---|---|
| | 14A |
| 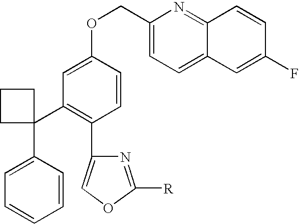 | |
| | 14B |
| 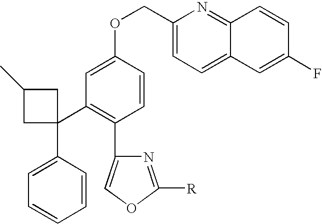 | |
| | 14C |
| 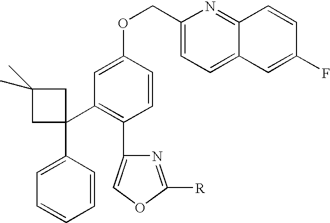 | |
| | 14D |
| 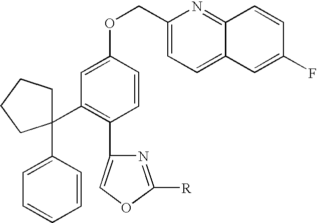 | |
| | 14E |
| 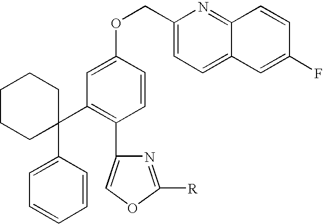 | |

| Ex. #14A | Ex. #14B* | Ex. #14C | Ex. #14D | Ex. #14E | R |
|---|---|---|---|---|---|
| a¹ | a¹ | a¹ | a¹ | a | H |
| b | b | b | b | b | CH₃ |
| c | c | c | c | c | CH₂CH3 |
| d | d | d | d | d | 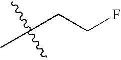 |
| e² | e² | e² | e² | e² | NH₂ |

¹Formamide is used in Step B.
²Urea is used in Step B.

In the examples shown above, the 6-fluoroquinoline group can also be replaced with quinoline, 5-fluoroquinoline, 5,6-difluoroquinoline, 5-fluoropyrazolo[1,5-a]pyridine, 6-fluoropyrazolo[1,5-a]pyridine, and 7-fluoropyrazolo[1,5-a]pyridine.

### Example 15

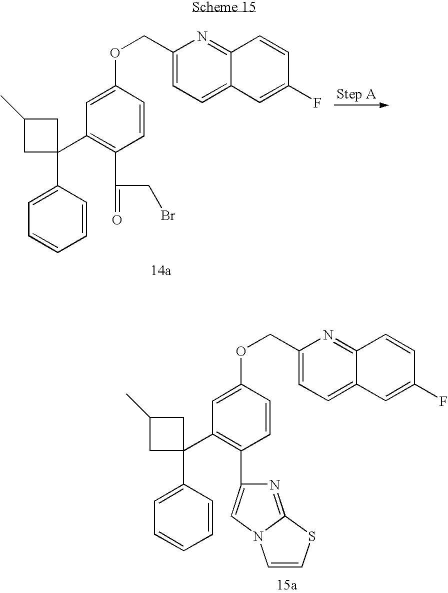

### Step A: Preparation of 6-fluoro-2-{[4-imidazo[2,1-b][1,3]thiazol-6-yl-3-(3-methyl-1-phenylcyclobutyl)phenoxy]methyl}quinoline (15a)

1,3-Thiazol-2-amine (1.05 equiv.) is added to a stirred solution of 14a (1 equiv.) in EtOH (0.05 M) at room temperature. The reaction mixture is sealed, heated to 78° C. and aged until the reaction is deemed complete. After cooling to room temperature, the volatiles are removed in vacuo and the residue is partitioned between EtOAc and sat. aq. sodium bicarbonate. The organic phase is separated and the aq, phase is extracted with EtOAc. The combined organic extracts are washed with water, brine, dried (MgSO4) and concentrated in vacuo. Purification of the crude residue by preparative TLC or flash chromatography affords the title compound 15a.

Following procedures similar to that described above for Example 15a, the following compounds (*compounds can be racemic or chiral) can be prepared:

| | |
|---|---|
| | 15A |
| 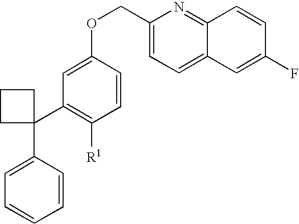 | |
| | 15B |
| 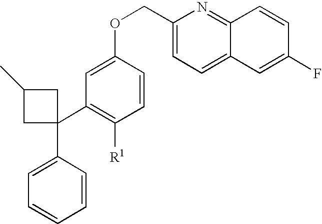 | |
| | 15C |
| 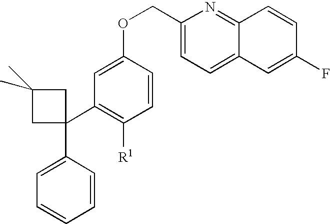 | |
| | 15D |
| 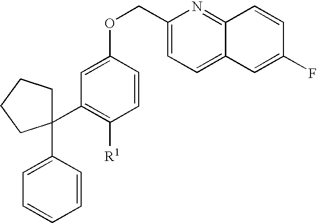 | |
| | 15E |
| 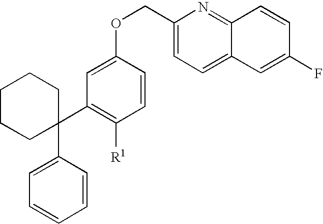 | |

| Ex. #15A | EX. #15B* | EX. #15C | EX. #15D | EX. #15E | R¹ |
|---|---|---|---|---|---|
| a | — | a | a | a | 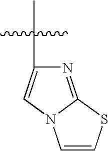 |
| b¹ | b¹ | b¹ | b¹ | b¹ | 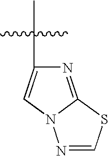 |
| c² | c² | c² | c² | c² | 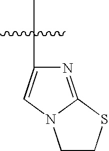 |
| d³ | d³ | d³ | d³ | d³ | 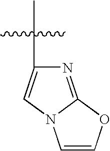 |

¹1,3,4-thiadiazol-2-amine is used in Step A.
²4,5-dihydro-1,3-thiazol-2-amine is used in Step A.
³1,3-oxazol-2-amine is used in Step A.

### Example 16

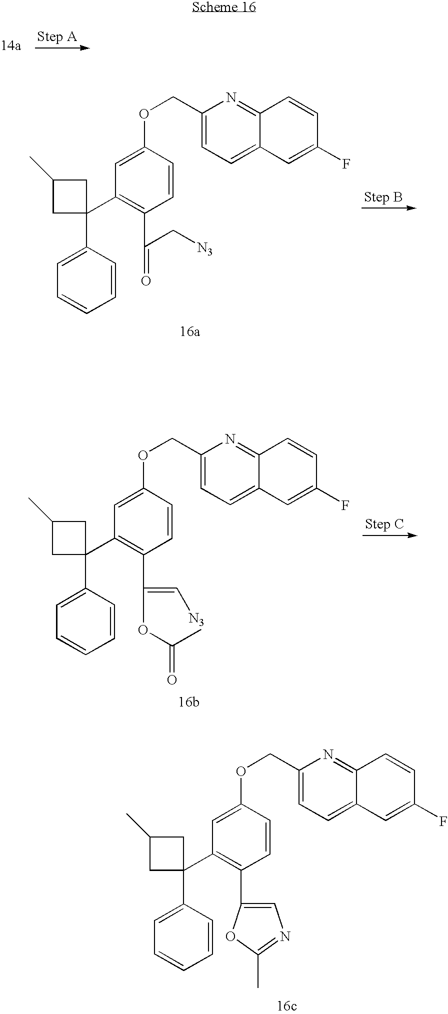

### Step A: Preparation of 2-azido-1-[4-[(6-fluoroquinolin-2-yl)methoxy]-2-(3-methyl-1-phenylcyclobutyl)phenyl]ethanone (16a)

Sodium azide (3.3 equiv.) is added to a stirred solution of 14a (1 equiv.) in DMF (0.1 M) at 0° C. After allowing to warm to room temperature, the reaction mixture is aged until the reaction is deemed complete. The reaction mixture is poured into water and extract three times with EtOAc. The combined organic extracts are washed with water, brine, dried (MgSO4) and concentrated in vacuo. Purification of the crude residue by flash chromatography provides the title compound 16a.

### Step B: Preparation of (Z)-2-azido-1-[4-[(6-fluoroquinolin-2-yl)methoxy]-2-(3-methyl-1-phenylcyclobutyl)phenyl]vinylacetate (16b)

Lithium diisopropylamide (1.2 equiv.) is added to a stirred solution of 16a (1 equiv.) in THF (0.05 M) at −78° C. After 5 min, acetic anhydride (1.2 equiv.) is added and the resulting mixture is stirred at −78° C. until the reaction is deemed complete. The reaction mixture is quenched with sat. aq. ammonium chloride and extracted three times with EtOAc. The combined organic extracts are washed with water, brine, dried (MgSO4) and concentrate in vacuo. Purification of the crude residue by flash chromatography affords the title compound 16b.

### Step C: Preparation of 6-fluoro-2-{[4-(2-methyl-1,3-oxazol-5-yl)-3-(3-methyl-1-phenylcyclobutyl)phenoxy]methyl}quinoline (16c)

Triethylphosphite (1.7 equiv.) is added dropwise to a stirred solution of 16b (1 equiv.) in cyclohexane (0.05 M) at room temperature. The resulting mixture is heated to 80° C. and aged until the reaction is deemed complete. After cooling to room temperature, the reaction mixture is concentrated in vacuo and the crude residue is purified by flash chromatography to furnish the title compound 16c.

Following procedures similar to that described above for Example 16c, the following compounds (*compounds can be racemic or chiral) can be prepared:

| | |
|---|---|
| | 16A |
| 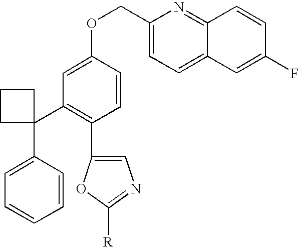 | |
| | 16B |
| 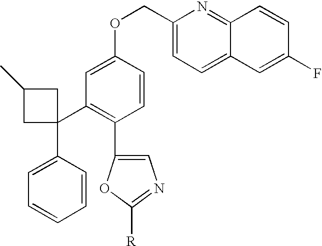 | |
| | 16C |
| 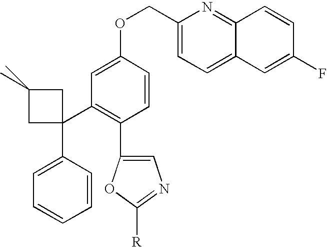 | |
| | 16D |
| 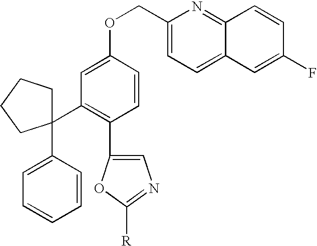 | |
| | 16E |
| 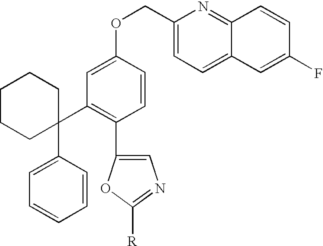 | |

| Ex. #16A | Ex. #16B | Ex. #16C | Ex. #16D | Ex. #16E | R |
|---|---|---|---|---|---|
| a | a | a | a | a | Me |
| b | b | b | b | b | Et |
| c¹ | c¹ | c¹ | c¹ | c¹ | CH₂OH |
| d² | d² | d² | d² | d² | CH₂F |
| e³ | e³ | e³ | e³ | e³ | 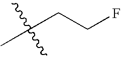 |

¹The alcohol functionality can be masked as a benzyl ether in Step B. Deprotection of the benzyl ether is achieved with methanesulfonic acid (according to J. Am. Chem. Soc., 1996, 118, 4560) to provide the desired alcohol product.
²Fluoroacetic anhydride derived from commercially available fluoracetic acid is used in Step B.
³3-Fluoropropanoic anhydride derived from commercially available 3-fluoropropanoic acid is used in Step B.

### FLAP Binding Assay

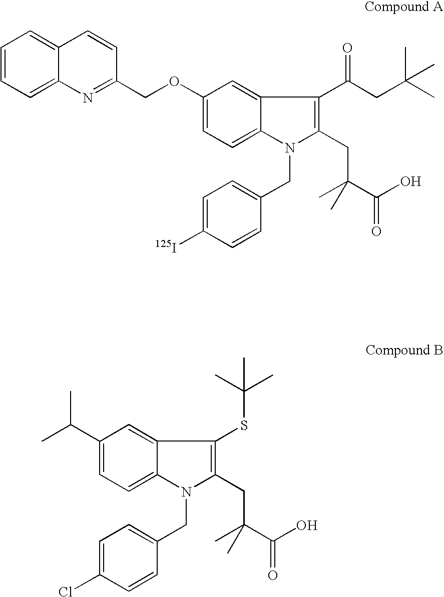

A 100,000×g pellet from human leukocyte 10,000×g supernatants (1) is the source of FLAP. The 100,000×g pellet membranes were resuspended in Tris-Tween assay buffer (100 mM Tris HCl pH 7.4, 140 mM NaCl, 2 mM EDTA, 0.5 mM dithiothreitol, 5% glycerol, 0.05% Tween 20) to yield a final protein concentration of 50 μg to 150 μg/ml. Aliquots (100 μl) of membrane suspension were added to 12 mm×75 mm polypropylene tubes containing 100 μl Tris-Tween assay buffer, 30,000 cpm of Compound A in 5 μl MeOH:assay buffer (1:1), and 2 μl dimethyl sulfoxide or competitor (i.e., the compound to be tested) in dimethyl sulfoxide. Compound B (10 μM final concentration) was used to determine non-specific binding. After a 20 minute incubation at room temperature, tube contents were diluted to 4 ml with cold 0.1 M Tris HCl pH 7.4, 0.05% Tween 20 wash buffer and the membranes were collected by filtration of GFB filters presoaked in the wash buffer. Tubes and filters were rinsed with 2×4 ml aliquots of cold wash buffer. Filters were transferred to 12 mm×3.5 mm polystyrene tubes for determination of radioactivity by gamma-scintillation counting.

Specific binding is defined as total binding minus non-specific binding. Total binding was Compound A bound to membranes in the absence of competitor; non-specific binding was Compound A bound in the presence of 10 uM Compound B. Preparation of Compound A is described in reference 1, below. The IC₅₀ values were obtained by computer analysis (see reference 2, below) of the experimental data. Representative tested compounds of the invention were determined to have an IC₅₀<1 uM, and preferred compounds had IC₅₀<200 nM.

### REFERENCES

1. Charleson, S., Prasti, P., Leger, S., Gillard, J. W, Vickers, P. J., Mancini, J. A., Charleson, P., Guay, J., Ford-Hutchinson, A. W., and Evans, J. F. (1992) Characterization of a 5-lipoxygenase-activating protein binding assay: correlation of affinity for 5-lipoxygenase-activating protein with leukotriene synthesis inhibition. Mol Pharmacol 41:873-879.
2. Kinetic, EBDA, Ligand, Lowry: A collection of Radioligand Binding Analysis Programs by G. A. McPherson. Elsevier-BIOSOFT.

While the invention has been described with reference to certain particular embodiments thereof, numerous alternative embodiments will be apparent to those skilled in the art from the teachings described herein. All patents, patent applications and publications cited herein are incorporated by reference in their entirety.

What is claimed:

1. A compound represented by Formula I: 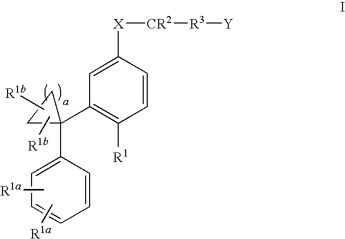
and the pharmaceutically acceptable salts, esters and solvates thereof wherein:
a is an integer selected from 1, 2, 3 and 4;
each R^{1a} is independently selected from the group consisting of: —H, —F, —Cl, —Br, —C₁₋₆alkyl, —CN, —OH, C₁₋₆alkyl-OH, —OC₁₋₆ alkyl, -fluoroC₁₋₆ alkyl, -fluoroC₁₋₆ alkoxy, —NH₂, —NHC₁₋₆alkyl, —N(C₁₋₆alkyl)₂, —C₁₋₆alkyl-NH₂, —C₁₋₆alkyl-NHC₁₋₆alkyl, —C₁₋₆alkyl-N(C₁₋₆alkyl)₂, —NHC(O)C₁₋₆alkyl, —CO₂C₁₋₆alkyl, —C(O)NHC₁₋₆alkyl and —C(O)N(C₁₋₆alkyl)₂;
each R^{1b} is independently selected from the group consisting of: —H, —F, —C₁₋₆ alkyl, —OH, —OC₁₋₆ alkyl, -fluoroC₁₋₆alkyl, -fluoroC₁₋₆alkoxy, —N(R^{a})₂ and —C₁₋₆alkyl-N(R^{a})₂,
or one R^{1b} group can represent oxo and the other is as previously defined;
R¹ is selected from the group consisting of:
a) Z¹,
b) —CO₂R^{a}, —C(O)NR^{a}R^{b}, —N(R^{a})₂, —NR^{b}SOₚR^{a}, —NR^{b}C(O)R^{a}, —NR^{b}C(O)NR^{a}R^{b}; —NR^{b}CO₂R^{a}, —OC(O)NR^{a}R^{b}, —OH and —CN,
c) —C₁₋₆alkyl, —C₂₋₆alkenyl, —C₂₋₆alkynyl, —OC₁₋₆alkyl, —OC₂₋₆alkenyl and —OC₂₋₆alkynyl, said groups being optionally substituted with R⁴ and optionally substituted with R⁵,
wherein R⁴ is selected from the group consisting of: —CO₂R^{a}, —C(O)NR^{a}R^{b}, —N(R^{a})₂, —NR^{b}SOₚR^{a}, —NR^{b}C(O)R^{a}, —NR^{b}C(O)NR^{a}R^{b}, —NR^{b}CO₂R^{a}, —OC(O)NR^{a}R^{b}, —C(O)SOₚNR^{a}R^{b}, —C(O)NR^{a}R^{b}, —S(O)ₚNR^{a}R^{b}, —SOₚNR^{a}R^{b}C(O)R^{a}, —S(O)ₚR^{a}, —F, —CF₃, phenyl, Hetcy and Z¹; and R⁵ is selected from the group consisting of —F and —OH, and
d) phenyl, optionally substituted with 1-2 members selected from the group consisting of: —F, —Cl, —C₁₋₆alkyl, —CN, —OH, —OC₁₋₆alkyl, -fluoroC₁₋₆alkyl, -fluoroC₁₋₆alkoxy, —NH₂, —NHC₁₋₆alkyl, —N(C₁₋₆alkyl)₂, —C₁₋₆alkyl-NH₂, —C₁₋₆alkyl-NHC₁₋₆alkyl, —C₁₋₆alkyl-N(C₁₋₆alkyl)₂, —C₁₋₆alkyl-CN, —NHC(O)C₁₋₆alkyl, —C(O)NHC₁₋₆alkyl and —C(O)N(C₁₋₆alkyl)₂;
R² is selected from the group consisting of —H and —C₁₋₆alkyl optionally substituted with a group selected from —OH and —F;
R³ is selected from the group consisting of —H and —C₁₋₆alkyl;
each “p” independently represents an integer selected from 0, 1 and 2;
each R^{a} is independently selected from the group consisting of
a) —H,
b) —C₁₋₄alkyl, —C₂₋₄alkenyl and —C₂₋₄alkyl, wherein each is optionally substituted with 1-2 members selected from the group consisting of: —OH, —OC₁₋₄alkyl, —CN, —NH₂, —NHC₁₋₄alkyl, and —N(C₁₋₄alkyl)₂, —F and —CF₃,
c) phenyl and phenyl-C₁₋₄alkyl-, the phenyl moieties being optionally substituted with 1-2 members selected from the group consisting of: —F, —Cl, —C₁₋₄ alkyl, —CN, —OH, —OC₁₋₄ alkyl, -fluoroC₁₋₄alkyl, -fluoroC₁₋₄alkoxy, —NH₂, —NHC₁₋₄alkyl, —N(C₁₋₄alkyl)₂, —C₁₋₄alkyl-NH₂, —C₁₋₄alkyl-NHC₁₋₄alkyl, —C₁₋₄alkyl-N(C₁₋₄alkyl)₂, —C₁₋₄alkyl-CN, —NHC(O)C₁₋₄alkyl, —C(O)NHC₁₋₄alkyl and —C(O)N(C₁₋₄alkyl)₂,
and the alkyl portion of phenyl-C₁₋₄alkyl- being optionally substituted with —OH, —CN, —OC₁₋₄alkyl, —NH₂, —NHC₁₋₄alkyl, —N(C₁₋₄alkyl)₂, and 1-3 of fluoro,
d) Hetcy and Hetcy-C₁₋₄alkyl-, the Hetcy moieties being optionally substituted on carbon with 1-2 members selected from the group consisting of —F, —OH, —CO₂H, —C₁₋₄alkyl, —CO₂C₁₋₄alkyl, —OC₁₋₄alkyl, —NH₂, —NHC₁₋₄alkyl, —N(C₁₋₄alkyl)₂, —NHC(O)C₁₋₄alkyl, oxo, —C(O)NHC₁₋₄alkyl and —C(O)N(C₁₋₄alkyl)₂; and optionally substituted on nitrogen when present with a group selected from —C₁₋₄alkyl and —C₁₋₄acyl,
and the alkyl portion of Hetcy-C₁₋₄alkyl- being optionally substituted with a member selected from the group consisting of —OH, —CN, —OC₁₋₄alkyl, —NH₂, —NHC₁₋₄alkyl, —N(C₁₋₄alkyl)₂ and 1-3 of fluoro,
e) Z² and Z²-C₁₋₄alkyl- and the alkyl portion of Z²-C₁₋₄alkyl- being optionally substituted with a member selected from the group consisting of —OH, —CN, —OC₁₋₄alkyl, —NH₂, —NHC₁₋₄alkyl, —N(C₁₋₄alkyl)₂ and 1-3 of fluoro;
each R^{b} is independently selected from the group consisting of —H and —C₁₋₃alkyl optionally substituted with 1-2 members selected from the group consisting of NH₂, —OH, —F, —CN and —CF₃;
X is selected from the group consisting of —O— and —CHR⁶—, wherein R⁶ is selected from the group consisting of —H, —OH and —C₁₋₆alkyl optionally substituted with a group selected from —OH and —F;
Y is selected from the group consisting of:
a) a 9-membered unsaturated ortho-fused bicyclic ring system containing 2-3 heteroatoms selected from the group consisting of —N═, —NH—, —N(Me)—, —S— and —O—, and wherein the ring system is optionally substituted with 1-3 of fluoro,
b) a 10-membered aromatic ortho-fused bicyclic ring system containing 1-3 of —N—, wherein the ring system is optionally substituted with 1-3 of fluoro, and
c) pyridinyl substituted with a group selected from —C₁₋₄alkyl, —F, —CF₂H and CF₃, and optionally having a second substituent which is —C₁₋₄alkyl;
Hetcy is selected from the group consisting of azetidinyl, pyrrolidinyl, piperidinyl, piperazinyl, morpholinyl, tetraydrofuranyl and β-lactamyl, δ-lactamyl and -γ-lactamyl;
Z¹ is selected from the group consisting of:
a) a 5-membered unsaturated heterocyclic ring containing 2-4 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro,
b) a 5-membered unsaturated heterocyclic ring containing 2-3 heteroatoms selected from one oxygen or one sulfur and 1-2 of nitrogen, wherein one nitrogen in the ring is optionally substituted with a group selected from C₁₋₄alkyl and C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro,
c) a 6-membered unsaturated heterocyclic ring containing 1-2 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro,
d) an 8-membered unsaturated ortho-fused bicyclic ring system containing 3-5 heteroatoms selected from one sulfur and 2-4 of nitrogen wherein one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro, and
e) a 9-membered unsaturated ortho-fused bicyclic ring system containing 3-4 nitrogen atoms, wherein one carbon in the ring is optionally substituted with a group selected from ═O, ═S, —SMe, —NH₂, —CF₃, —Cl, —C₁₋₄alkyl and C₁₋₄alkyl substituted with a group selected from —NH₂, —OH, —OC₁₋₄alkyl, —CN and 1-3 of fluoro; and
Z² is selected from the group consisting of:
a) a 5-membered unsaturated heterocyclic ring containing 2-4 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro,
b) a 5-membered unsaturated heterocyclic ring containing 2-3 heteroatoms selected from one oxygen or one sulfur and 1-2 of nitrogen, wherein one nitrogen in the ring is optionally substituted with a group selected from C₁₋₄alkyl and C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, and C₁₋₄alkyl optionally substituted with a group selected from —NH₂, —OH, —OC₁₋₄alkyl, —CN and 1-3 of fluoro, and
c) a 6-membered unsaturated heterocyclic ring containing 1-2 nitrogen atoms, wherein one nitrogen in the ring is optionally substituted with a group selected from —C₁₋₄alkyl and —C₁₋₄alkyl substituted with a group selected from —NH₂, —OH, —CN and 1-3 of fluoro, and one carbon in the ring is optionally substituted with a group selected from ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro.

2. The compound of claim 1 wherein Y is selected from the group consisting of: 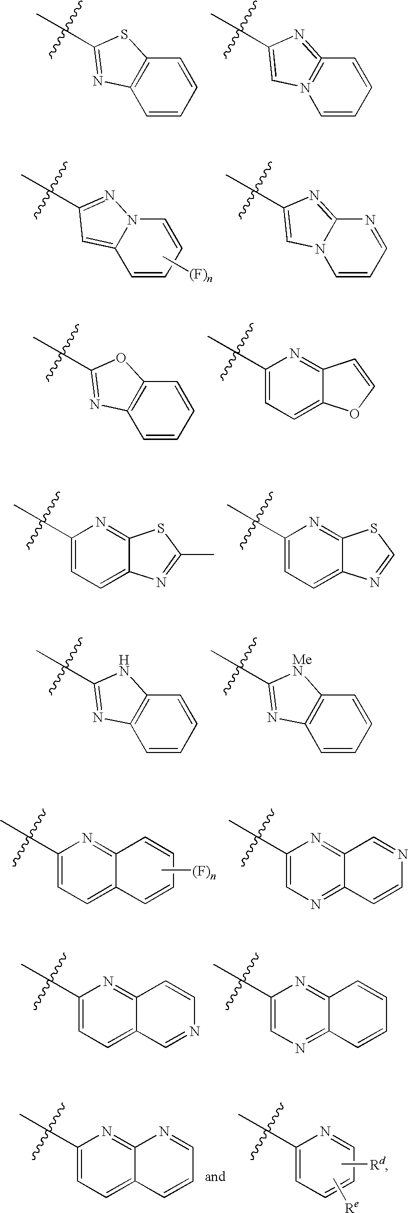
wherein R^{d} is selected from —C₁₋₄alkyl, —F, —CF₂H and —CF₃; R^{e} is selected from —H and —C₁₋₄alkyl; and n is an integer selected from zero, 1, 2 and 3.

3. The compound of claim 2 wherein R¹ is selected from —COOH, —COOC₁₋₃alkyl, —C(O)—NR^{a}R^{b}, —OC(O)—NR^{a}R^{b}, —CH₂C(O)—NR^{a}R^{b} and Z¹.

4. The compound of claim 3 wherein X is —O—.

5. The compound of claim 4 wherein Z¹ is selected from the group consisting of: 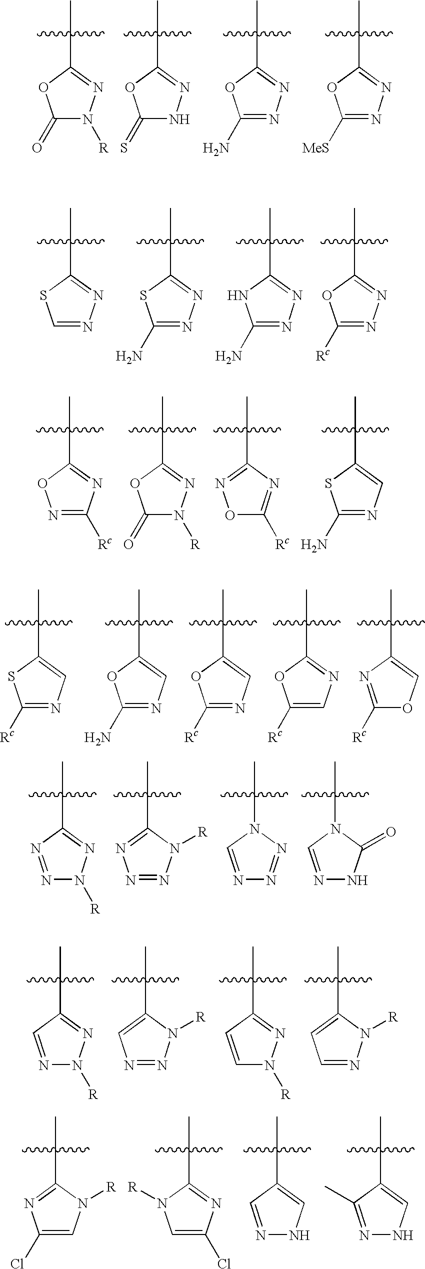 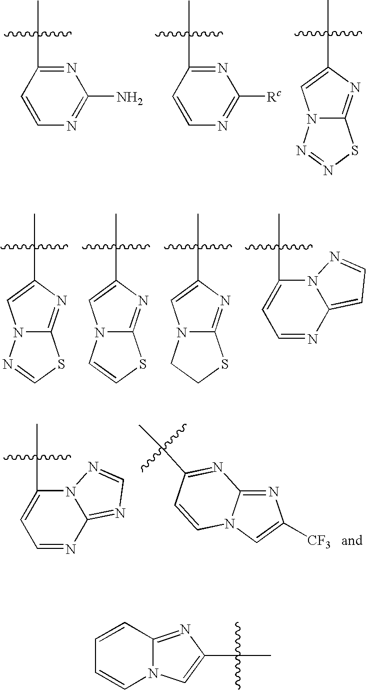
wherein R is selected from —H, —C₁₋₄alkyl and —C₁₋₄alkyl substituted with a group selected from —NH₂, —OH, —CN and 1-3 of fluoro, and R^{c}C is selected from —H, ═O, ═S, —SMe, —NH₂, —CF₃, —Cl, —C₁₋₄alkyl and —C₁₋₄alkyl substituted with a group selected from —NH₂, —OH, —OC₁₋₄alkyl, —CN and 1-3 of fluoro.

6. The compound of claim 5 wherein R^{a} is selected from —H and Z², and R^{b} is selected from —H, methyl, ethyl, propyl and i-propyl.

7. The compound of claim 6 wherein Z² is selected from pyridinyl, pyrimidinyl, pyrazinyl, thiazolyl, thiadiazolyl, triazolyl and pyrazolyl, each optionally substituted.

8. The compound of claim 7 wherein R⁴ is selected from —H, —CONR^{a}R^{b}, —OCONR^{a}R^{b} and —CO₂R^{a}.

9. The compound of claim 8 wherein a is selected from 2, 3 and 4;
each R^{1a} is independently selected from —H and —F; each R^{1b} is independently selected from —H and —CH₃; R² is —H; R³ is —H; and Hetcy is selected from pyrrolidinyl and piperidinyl.

10. The compound of claim 1 of structural Formula Ia 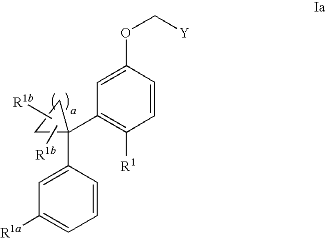
and the pharmaceutically acceptable salts, esters and solvates thereof.

11. The compound of claim 1 of structural formula Ib 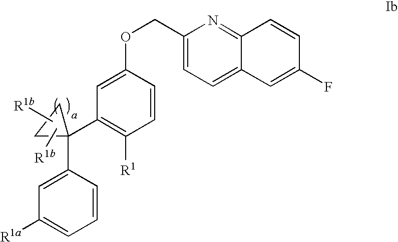
and the pharmaceutically acceptable salts, esters and solvates thereof.

12. A pharmaceutical composition comprised of a therapeutically effective amount of a compound of claim 1 and a pharmaceutically acceptable carrier.

13. A method for treating a leukotriene-mediated medical condition comprising administering a therapeutically effective amount of a compound of claim 1 to a patient in need of such treatment.

14. A method for treating an inflammatory condition comprising administering a therapeutically effective amount of a compound of claim 1 to a patient in need of such treatment.

15. A method for treating atherosclerosis comprising administering a therapeutically effective amount of a compound of claim 1 to a patient in need of such treatment.

16. The method of claim 15 for halting or slowing atherosclerotic plaque progression.

17. The method of claim 15 for effecting regression of atherosclerotic plaque.

18. The method of claim 15 for preventing or reducing the risk of atherosclerotic plaque rupture in a patient having atherosclerotic plaque.

19. A method for preventing or reducing the risk of an atherosclerotic disease event comprising administering a prophylactically effective amount of a compound of claim 1 to a patient at risk for having an atherosclerotic disease event.

20. The method of treating atherosclerosis of claim 19 further comprising administering to the patient a compound selected from the group consisting of an HMG-CoA reductase inhibitor, cholesterol absorption inhibitor, CETP inhibitor, PPARγ agonist, PPARα agonist, PPAR dual α/γ agonist, and combinations thereof.
